SEQUENCE LISTING (234 sequences)

SEQ ID NO: 1 - DNA, 23 bp, Artificial Sequence
  CodY target sequence
atgttcagaaaattcatgaacat

SEQ ID NO: 2 - DNA, 19 bp, Artificial Sequence
  primer sto 14
cttgccatggaatcacccg

SEQ ID NO: 3 - DNA, 28 bp, Artificial Sequence
  primer opp1
gctctagacactcacttgttttgcttcc

SEQ ID NO: 4 - DNA, 27 bp, Artificial Sequence
  primer opp2
aactgcaggaaaattcatgaacatacc

SEQ ID NO: 5 - DNA, 28 bp, Artificial Sequence
  primer opp3
aactgcagtaaaacaataataaaagcag

SEQ ID NO: 6 - DNA, 27 bp, Artificial Sequence
  primer opp4
aactgcaggataataaaatttggactg

SEQ ID NO: 7 - DNA, 27 bp, Artificial Sequence
  primer opp14
aactgcagcgtaatgttcagaaaattc

SEQ ID NO: 8 - DNA, 38 bp, Artificial Sequence
  primer opp15 (a)
aactgcagcgtaatatttagaaaattcatgaacatacc

SEQ ID NO: 9 - DNA, 38 bp, Artificial Sequence
  primer opp15 (b)
aactgcagcgtactgtgccgaaaattcatgaacatacc

SEQ ID NO: 10 - DNA, 27 bp, Artificial Sequence
  primer cod280A
gggaattcggattgtctatctgcctcg

SEQ ID NO: 11 - DNA, 36 bp, Artificial Sequence
  primer cod280B
gggggatccagatctgaccatgattacgccaagctt

SEQ ID NO: 12 - DNA, 60 bp, Artificial Sequence
  primer HC-5
ctagaccaccatggggcatcaccatcaccatcacgtggctacattacttgaaaaaacacg

SEQ ID NO: 13 - DNA, 37 bp, Artificial Sequence
  primer HC-6
ctagtctagattagaaattacgtccagcaagtttatc

SEQ ID NO: 14 - DNA, 15 bp, Artificial Sequence
  inversely repeated (IR) cis-element
aattttcwgaaaatt

SEQ ID NO: 15 - protein, 8 aa, Artificial Sequence
  consensus sequence GTP binding motif in CodY homologs
  Xaa (2)..(2): Arg
  Xaa (3)..(3): Met, Gly, Ile, Lys, Gln
  Xaa (4)..(4): Gly
  Xaa (5)..(5): Thr
  Xaa (7)..(7): Ser
GGERLGTT

SEQ ID NO: 16 - protein, 8 aa, Escherichia coli
  BINDING (1)..(8): /note=“Putative GTP binding motif G1”
LGGGTGTG

SEQ ID NO: 17 - protein, 8 aa, Bacillus subtilis
  BINDING (1)..(8): /note=“Putative GTP binding motif G1”
GGERLGTL

SEQ ID NO: 18 - protein, 8 aa, Bacillus halodurans
  BINDING (1)..(8): /note=“Putative GTP binding motif G1”
GGQRLGTL

SEQ ID NO: 19 - protein, 8 aa, Clostridium difficile
  BINDING (1)..(8): /note=“Putative GTP binding motif G1”
GGMRLGSL

SEQ ID NO: 20 - protein, 8 aa, Clostridium acetobutylicum
  BINDING (1)..(8): /note=“Putative GTP binding motif G1”
NRERLGTL

SEQ ID NO: 21 - protein, 8 aa, Streptococcus pneumoniae
  BINDING (1)..(8): /note=“Putative GTP binding motif G1”
SGIRLGSL

SEQ ID NO: 22 - protein, 8 aa, Enterococcus faecalis
  BINDING (1)..(8): /note=“Putative GTP binding motif G1”
AGKRLGTI

SEQ ID NO: 23 - protein, 8 aa, Lactococcus lactis
  BINDING (1)..(8): /note=“Putative GTP binding motif G1”
SGMRLGTF

SEQ ID NO: 24 - protein, 4 aa, Artificial Sequence
  consensus sequence GTP binding domain in CodY homologs
  Variant (2)..(2): Ala, Lys
  Variant (3)..(3): Phe, Ile
DRVG

SEQ ID NO: 25 - protein, 4 aa, Escherichia coli
  BINDING (1)..(4): /note=“Putative GTP binding motif G3”
DAFG

SEQ ID NO: 26 - protein, 4 aa, Bacillus subtilis
  BINDING (1)..(4): /note=“Putative GTP binding motif G3”
DRVG

SEQ ID NO: 27 - protein, 4 aa, Clostridium difficile
  BINDING (1)..(4): /note=“Putative GTP binding motif G3”
DRIG

SEQ ID NO: 28 - protein, 4 aa, Lactococcus lactis
  BINDING (1)..(4): /note=“Putative GTP binding motif G3”
DKIG

SEQ ID NO: 29 - protein, 4 aa, Artificial Sequence
  consensus sequence GTP motif in CodY homologs
  BINDING (1)..(4): /note=“Putative GTP binding motif G4”
  VARIANT (3)..(3): Leu, Phe, Asn, Ser, Gln
NKGD

SEQ ID NO: 30 - protein, 4 aa, Escherichia coli
  BINDING (1)..(4): /note=“Putative GTP binding motif G4”
TSLD

SEQ ID NO: 31 - protein, 4 aa, Bacillus subtilis
  BINDING (1)..(4): /note=“Putative GTP binding motif G4”
NKFL

SEQ ID NO: 32 - protein, 4 aa, Bacillus stearothermophilus
  BINDING (1)..(4): /note=“Putative GTP binding motif G4”
DKFL

SEQ ID NO: 33 - protein, 4 aa, Clostridium difficile
  BINDING (1)..(4): /note=“Putative GTP binding motif G4”
NEGI

SEQ ID NO: 34 - protein, 4 aa, Clostridium acetobutylicum
  BINDING (1)..(4): /note=“Putative GTP binding motif G4”
ILND

SEQ ID NO: 35 - protein, 4 aa, Streptococcus pneumoniae
  BINDING (1)..(4): /note=“Putative GTP binding motif G4”
LISD

SEQ ID NO: 36 - protein, 4 aa, Enterococcus faecalis
  BINDING (1)..(4): /note=“Putative GTP binding motif G4”
NQQF

SEQ ID NO: 37 - protein, 4 aa, Staphylococcus aureus
  BINDING (1)..(4): /note=“Putative GTP binding motif G4”
EKGI

SEQ ID NO: 38 - protein, 4 aa, Lactococcus lactis
  BINDING (1)..(4): /note=“Putative GTP binding motif G4”
TGLF

SEQ ID NO: 39 - DNA, 26 bp, Artificial Sequence
  presence of the putative CodY box in yreE
taattttctgataatatagtcaattt

SEQ ID NO: 40 - DNA, 26 bp, Artificial Sequence
  presence of the putative CodY box in ctrA
taatttactgacaagtctgtcagtaa

SEQ ID NO: 41 - DNA, 26 bp, Artificial Sequence
  presence of the putative CodY box in yciC
taatttactgacaaaattatcagaac

SEQ ID NO: 42 - DNA, 26 bp, Artificial Sequence
  presence of the putative CodY box in optA
aaattttctgacaataataaaaattg

SEQ ID NO: 43 - DNA, 26 bp, Artificial Sequence
  presence of the putative CodY box in optS
aaattatcagaaaaatacaacaatat

SEQ ID NO: 44 - DNA, 26 bp, Artificial Sequence
  presence of the putative CodY box in optS
taattttcagaataatatgaaaattc

SEQ ID NO: 45 - DNA, 26 bp, Artificial Sequence
  presence of the putative CodY box in parA
taatttactgatagatttgtcagtaa

SEQ ID NO: 46 - DNA, 26 bp, Artificial Sequence
  presence of the putative CodY box in aroF
taatttactgacagttctgtcagtaa

SEQ ID NO: 47 - DNA, 26 bp, Artificial Sequence
  presence of the putative CodY box in vacB1
aaatttactgacaaaaaagataatgg

SEQ ID NO: 48 - DNA, 26 bp, Artificial Sequence
  presence of the putative CodY box in optA
taattttcagaaaacataaccattat

SEQ ID NO: 49 - DNA, 26 bp, Artificial Sequence
  presence of the putative CodY box in ypaG
gaattttatgaaaaaaatattaattg

SEQ ID NO: 50 - DNA, 26 bp, Artificial Sequence
  presence of the putative CodY box in yiaB
gaatttactgacgaatctatcattaa

SEQ ID NO: 51 - DNA, 26 bp, Artificial Sequence
  presence of the putative CodY box in ysdC
tcattctctgacaaatctgtcagtaa

SEQ ID NO: 52 - DNA, 26 bp, Artificial Sequence
  presence of the putative CodY box in hemK
aaatttactgacaagcttgttagtat

SEQ ID NO: 53 - DNA, 26 bp, Artificial Sequence
  presence of the putative CodY box in prfC
aaatttaatgataaaacaattagttt

SEQ ID NO: 54 - DNA, 26 bp, Artificial Sequence
  presence of the putative CodY box in yugB
aaagttactgacaaatctgtcagtaa

SEQ ID NO: 55 - DNA, 26 bp, Artificial Sequence
  presence of the putative CodY box in murD
ttatttactgacaagtctgtcagtaa

SEQ ID NO: 56 - DNA, 26 bp, Artificial Sequence
  presence of the putative CodY box in ywdG
tattttactgacaaaaaaataagttt

SEQ ID NO: 57 - DNA, 26 bp, Artificial Sequence
  presence of the putative CodY box in parC
taatttactgacagctttgtcagtaa

SEQ ID NO: 58 - DNA, 26 bp, Artificial Sequence
  presence of the putative CodY box in pepC
aaatttactgacagagctgtcagtaa

SEQ ID NO: 59 - DNA, 26 bp, Artificial Sequence
  presence of the putative CodY box in mutM
aaatttactgacagacttgttagtaa

SEQ ID NO: 60 - DNA, 26 bp, Artificial Sequence
  presence of the putative CodY box in rgpAB
aaatttactgacaactttgtcagaag

SEQ ID NO: 61 - DNA, 26 bp, Artificial Sequence
  presence of the putative CodY box in vacB1
aaaatgtctgataaaatgattaatac

SEQ ID NO: 62 - DNA, 26 bp, Artificial Sequence
  presence of the putative CodY box in recN
taatttactgacagaattttaaattt

SEQ ID NO: 63 - DNA, 26 bp, Artificial Sequence
  presence of the putative CodY box in llrH
aaaattactaacaaaactgttagtaa

SEQ ID NO: 64 - DNA, 15 bp, Artificial Sequence
  motif in L. lactis MG1363 genome
aattttcagaaaatt

SEQ ID NO: 65 - DNA, 15 bp, Artificial Sequence
  motif in L. lactis MG1363 genome
aattgtcagaaaatt

SEQ ID NO: 66 - DNA, 15 bp, Artificial Sequence
  motif in L. lactis MG1363 genome
aattttctgataatt

SEQ ID NO: 67 - DNA, 15 bp, Artificial Sequence
  motif in L. lactis MG1363 genome
aattttcagataatt

SEQ ID NO: 68 - DNA, 15 bp, Artificial Sequence
  motif in L. lactis MG1363 genome
atttttcagaaaatt

SEQ ID NO: 69 - DNA, 15 bp, Artificial Sequence
  motif in L. lactis MG1363 genome
aattttcggaaaaat

SEQ ID NO: 70 - DNA, 15 bp, Artificial Sequence
  motif in L. lactis MG1363 genome
aattttcagaaaata

SEQ ID NO: 71 - DNA, 15 bp, Artificial Sequence
  motif in L. lactis MG1363 genome
acttttcagaaaatt

SEQ ID NO: 72 - DNA, 15 bp, Artificial Sequence
  motif in L. lactis MG1363 genome
aattttctgaatatt

SEQ ID NO: 73 - DNA, 15 bp, Artificial Sequence
  motif in L. lactis MG1363 genome
atttttcagaaaaat

SEQ ID NO: 74 - DNA, 15 bp, Artificial Sequence
  motif in L. lactis MG1363 genome
aattgtcagacaatt

SEQ ID NO: 75 - DNA, 15 bp, Artificial Sequence
  motif in L. lactis MG1363 genome
atttttctgacaatt

SEQ ID NO: 76 - DNA, 15 bp, Artificial Sequence
  motif in L. lactis MG1363 genome
aattttctgaacatt

SEQ ID NO: 77 - DNA, 15 bp, Artificial Sequence
  motif in L. lactis MG1363 genome
atttttccgaaaatt

SEQ ID NO: 78 - DNA, 15 bp, Artificial Sequence
  motif in L. lactis MG1363 genome
tattttcaaaaaatt

SEQ ID NO: 79 - DNA, 15 bp, Artificial Sequence
  motif in L. lactis MG1363 genome
attattctgaaaatt

SEQ ID NO: 80 - DNA, 15 bp, Artificial Sequence
  motif in L. lactis MG1363 genome
tattttcaaaaaatt

SEQ ID NO: 81 - DNA, 15 bp, Artificial Sequence
  motif in L. lactis MG1363 genome
aattttttgaaaatt

SEQ ID NO: 82 - DNA, 15 bp, Artificial Sequence
  motif in L. lactis MG1363 genome
aaatttctgaaaatt

SEQ ID NO: 83 - DNA, 15 bp, Artificial Sequence
  motif in L. lactis MG1363 genome
aattttctgacaata

SEQ ID NO: 84 - DNA, 15 bp, Artificial Sequence
  motif in L. lactis MG1363 genome
atttttctgaaaata

SEQ ID NO: 85 - DNA, 15 bp, Artificial Sequence
  motif in L. lactis MG1363 genome
atttttcagaaaata

SEQ ID NO: 86 - DNA, 15 bp, Artificial Sequence
  motif in L. lactis MG1363 genome
gtttttctgaaaatt

SEQ ID NO: 87 - DNA, 15 bp, Artificial Sequence
  motif in L. lactis MG1363 genome
aattttcagaagaat

SEQ ID NO: 88 - DNA, 15 bp, Artificial Sequence
  motif in L. lactis MG1363 genome
aattttctaaaaagt

SEQ ID NO: 89 - DNA, 15 bp, Artificial Sequence
  motif in L. lactis MG1363 genome
aattttcagaaaaca

SEQ ID NO: 90 - DNA, 15 bp, Artificial Sequence
  motif in L. lactis MG1363 genome
tattttcaaaaaaat

SEQ ID NO: 91 - DNA, 15 bp, Artificial Sequence
  motif in L. lactis MG1363 genome
catcttcagaaaatt

SEQ ID NO: 92 - DNA, 15 bp, Artificial Sequence
  motif in L. lactis MG1363 genome
aatattcagaaaata

SEQ ID NO: 93 - DNA, 15 bp, Artificial Sequence
  motif in L. lactis MG1363 genome
aattttcaaaaaact

SEQ ID NO: 94 - DNA, 15 bp, Artificial Sequence
  motif in L. lactis MG1363 genome
aattttcagaaaaga

SEQ ID NO: 95 - DNA, 15 bp, Artificial Sequence
  motif in L. lactis MG1363 genome
aatgttctgacaaat

SEQ ID NO: 96 - DNA, 15 bp, Artificial Sequence
  motif in L. lactis MG1363 genome
aattttttgaaaaat

SEQ ID NO: 97 - DNA, 15 bp, Artificial Sequence
  motif in L. lactis MG1363 genome
aattttctaaaaact

SEQ ID NO: 98 - DNA, 15 bp, Artificial Sequence
  motif in L. lactis MG1363 genome
attttacagaaaatt

SEQ ID NO: 99 - DNA, 15 bp, Artificial Sequence
  motif in L. lactis MG1363 genome
aattatcagaaaatg

SEQ ID NO: 100 - DNA, 15 bp, Artificial Sequence
  motif in L. lactis MG1363 genome
aattatcagaaaatc

SEQ ID NO: 101 - DNA, 15 bp, Artificial Sequence
  motif in L. lactis MG1363 genome
atttttctgaaaatg

SEQ ID NO: 102 - DNA, 15 bp, Artificial Sequence
  motif in L. lactis MG1363 genome
aattttctgacgatt

SEQ ID NO: 103 - DNA, 15 bp, Artificial Sequence
  presence of putative CodY box in yufN
attatcagaaaattt

SEQ ID NO: 104 - DNA, 15 bp, Artificial Sequence
  presence of putative CodY box in citB
attgtgagaaaattg

SEQ ID NO: 105 - DNA, 15 bp, Artificial Sequence
  presence of putative CodY box in dppA
tttgttagaatattc

SEQ ID NO: 106 - DNA, 15 bp, Artificial Sequence
  presence of putative CodY box in hutP
gttatcagaattttt

SEQ ID NO: 107 - DNA, 15 bp, Artificial Sequence
  presence of putative CodY box in yxbC
attatcagaggatta

SEQ ID NO: 108 - DNA, 15 bp, Artificial Sequence
  presence of putative CodY box in yurPQ
aatttcagaaaataa

SEQ ID NO: 109 - DNA, 15 bp, Artificial Sequence
  presence of putative CodY box in ycgM
attttgaggatattg

SEQ ID NO: 110 - DNA, 15 bp, Artificial Sequence
  presence of putative CodY box in yhjC
aatttcagacaattc

SEQ ID NO: 111 - DNA, 15 bp, Artificial Sequence
  presence of putative CodY box in ybgE
atattctgaaattta

SEQ ID NO: 112 - DNA, 15 bp, Artificial Sequence
  presence of putative CodY box in ykbA
tttatcaaaaaagtc

SEQ ID NO: 113 - DNA, 15 bp, Artificial Sequence
  presence of putative CodY box in ggaA
attttcagcaaaaaa

SEQ ID NO: 114 - DNA, 15 bp, Artificial Sequence
  presence of putative CodY box in ycgM
ataatcagaatcttt

SEQ ID NO: 115 - DNA, 15 bp, Artificial Sequence
  presence of putative CodY box in yoaD
tttttatgaaaaata

SEQ ID NO: 116 - DNA, 15 bp, Artificial Sequence
  presence of putative CodY box in guaB
gttatctaaatattt

SEQ ID NO: 117 - DNA, 15 bp, Artificial Sequence
  presence of putative CodY box in ilvD
attgtcaaaataaaa

SEQ ID NO: 118 - DNA, 15 bp, Artificial Sequence
  presence of putative CodY box in yxbBC
attgacagaattatc

SEQ ID NO: 119 - DNA, 15 bp, Artificial Sequence
  presence of putative CodY box in rocA
tttttcagcaaaaga

SEQ ID NO: 120 - DNA, 15 bp, Artificial Sequence
  presence of putative CodY box in yhdG
tttttctaacaattt

SEQ ID NO: 121 - DNA, 15 bp, Artificial Sequence
  presence of putative CodY box in yusC
ttttgcagaaaaaac

SEQ ID NO: 122 - DNA, 15 bp, Artificial Sequence
  presence of putative CodY box in yusC
tttcttagaatagtg

SEQ ID NO: 123 - DNA, 15 bp, Artificial Sequence
  presence of the putative CodY box in ydjJ
attttcagaaattta

SEQ ID NO: 124 - DNA, 15 bp, Artificial Sequence
  presence of the putative CodY box in ytkC
tttttcagaaaaatg

SEQ ID NO: 125 - DNA, 15 bp, Artificial Sequence
  presence of the putative CodY box in lytE
tttttcagaaaaatc

SEQ ID NO: 126 - DNA, 15 bp, Artificial Sequence
  presence of the putative CodY box in yoaC
attgtctgaatatta

SEQ ID NO: 127 - DNA, 15 bp, Artificial Sequence
  presence of the putative CodY box in ytnA
tttttctgaatattc

SEQ ID NO: 128 - DNA, 15 bp, Artificial Sequence
  presence of the putative CodY box in aldY
attttcggaaaattt

SEQ ID NO: 129 - DNA, 15 bp, Artificial Sequence
  presence of the putative CodY box in glnQ
attttcagaaaagtt

SEQ ID NO: 130 - DNA, 15 bp, Artificial Sequence
  presence of the putative CodY box in ykuW
attttcagaaaataa

SEQ ID NO: 131 - DNA, 15 bp, Artificial Sequence
  presence of the putative CodY box in yndN
attttcagtatattt

SEQ ID NO: 132 - DNA, 15 bp, Artificial Sequence
  presence of the putative CodY box in yheI
tttatcagaaaaata

SEQ ID NO: 133 - DNA, 15 bp, Artificial Sequence
  presence of the putative CodY box in ykuM
attttcaggaaattc

SEQ ID NO: 134 - DNA, 15 bp, Artificial Sequence
  presence of the putative CodY box in gbsA
attttcagaacaatt

SEQ ID NO: 135 - DNA, 15 bp, Artificial Sequence
  presence of the putative CodY box in ynaC
attttcagaaaatca

SEQ ID NO: 136 - DNA, 15 bp, Artificial Sequence
  presence of the putative CodY box in yqiQ
attgtcagaaaactt

SEQ ID NO: 137 - DNA, 15 bp, Artificial Sequence
  presence of the putative CodY box in ggaA
attttcagaattata

SEQ ID NO: 138 - DNA, 15 bp, Artificial Sequence
  presence of the putative CodY box in yurY
tttttcggaatattc

SEQ ID NO: 139 - DNA, 15 bp, Artificial Sequence
  presence of the putative CodY box in ureA
tttatctgaaaattt

SEQ ID NO: 140 - DNA, 15 bp, Artificial Sequence
  presence of the putative CodY box in leuS
atattcagaatattc

SEQ ID NO: 141 - DNA, 15 bp, Artificial Sequence
  presence of the putative CodY box in yurO
attttctgaaatttc

SEQ ID NO: 142 - DNA, 15 bp, Artificial Sequence
  presence of the putative CodY box in ykrQ
attttctgaaattta

SEQ ID NO: 143 - DNA, 15 bp, Artificial Sequence
  presence of the putative CodY box in yvcC
tttttcagtatattt

SEQ ID NO: 144 - DNA, 15 bp, Artificial Sequence
  presence of the putative CodY box in thiC
tttttcagcaaattt

SEQ ID NO: 145 - DNA, 15 bp, Artificial Sequence
  presence of the putative CodY box in yvaV
tttttcagacaattg

SEQ ID NO: 146 - DNA, 15 bp, Artificial Sequence
  presence of the putative CodY box in fliR
attgtcagcatattt

SEQ ID NO: 147 - DNA, 15 bp, Artificial Sequence
  presence of the putative CodY box in yokA
tttttcataaaattt

SEQ ID NO: 148 - DNA, 15 bp, Artificial Sequence
  presence of the putative CodY box in yqeY
tttttcataaaattc

SEQ ID NO: 149 - DNA, 15 bp, Artificial Sequence
  presence of the putative CodY box in yjcL
  misc_feature (1)..(15)
attgttagaaaatta

SEQ ID NO: 150 - DNA, 15 bp, Artificial Sequence
  presence of the putative CodY box in rapE
  misc_feature (1)..(15)
actttcagaatattt

SEQ ID NO: 151 - DNA, 15 bp, Artificial Sequence
  presence of the putative CodY box in bfmBB
tttttctgaataatt

SEQ ID NO: 152 - DNA, 26 bp, Artificial Sequence
  presence of the putative CodY box in ppmA
tgattttcagaaaaatttaagaaaaa

SEQ ID NO: 153 - DNA, 26 bp, Artificial Sequence
  presence of the putative CodY box in Leu-A
atattttctgaaaatttcttcagtaa

SEQ ID NO: 154 - DNA, 26 bp, Artificial Sequence
  presence of the putative CodY box in hsds
ttattatcagataattttatcaatcg

SEQ ID NO: 155 - DNA, 26 bp, Artificial Sequence
  presence of the putative CodY box in livJ
caattttctgataattcggtatattc

SEQ ID NO: 156 - DNA, 26 bp, Artificial Sequence
  presence of the putative CodY box in ilvE
gaattttctgaaaattacaaaatata

SEQ ID NO: 157 - DNA, 26 bp, Artificial Sequence
  presence of the putative CodY box in gapN
ttattttctgaaaatttggtaaaata

SEQ ID NO: 158 - DNA, 26 bp, Artificial Sequence
  presence of the putative CodY box in mefE
tcattttttgaaaaaatgattattac

SEQ ID NO: 159 - DNA, 26 bp, Artificial Sequence
  presence of the putative CodY box in IS1381
catttttcagaaaattcttttatttc

SEQ ID NO: 160 - DNA, 26 bp, Artificial Sequence
  presence of the putative CodY box in ugd
acattttctgaaattaaaaataatat

SEQ ID NO: 161 - DNA, 26 bp, Artificial Sequence
  presence of the putative CodY box in hemK
gaaatttctgaaaaatatgatataat

SEQ ID NO: 162 - DNA, 26 bp, Artificial Sequence
  presence of the putative CodY box in spr1649
aaattatttgataattctataatttc

SEQ ID NO: 163 - DNA, 26 bp, Artificial Sequence
  presence of the putative CodY box in spr1765
aatttatctgaaaaaacgaaaaatat

SEQ ID NO: 164 - DNA, 26 bp, Artificial Sequence
  presence of the putative CodY box in glgB
caattttttgaaaaaatattgattta

SEQ ID NO: 165 - DNA, 26 bp, Artificial Sequence
  presence of the putative CodY box in spr2010
tatttttctgaaaactctgatataaa

SEQ ID NO: 166 - DNA, 26 bp, Artificial Sequence
  presence of the putative CodY box in spr0128
tcattttcagataaggataaaaattg

SEQ ID NO: 167 - DNA, 26 bp, Artificial Sequence
  presence of the putative CodY box in ABC-NDB
gaattgacagatgaatttgttaagaa

SEQ ID NO: 168 - DNA, 26 bp, Artificial Sequence
  presence of the putative CodY box in spr1149
aaattgaatgaaaagtataaaattaa

SEQ ID NO: 169 - DNA, 26 bp, Artificial Sequence
  presence of the putative CodY box in rgg/spr1934
aaattgtcagaattatgagaaaatag

SEQ ID NO: 170 - DNA, 26 bp, Artificial Sequence
  presence of the putative CodY box in gidA
cattttactgaagaatacgatattat

SEQ ID NO: 171 - DNA, 26 bp, Artificial Sequence
  presence of the putative CodY box in abc-n/p
aaatttctagataagttaataattaa

SEQ ID NO: 172 - DNA, 26 bp, Artificial Sequence
  presence of the putative CodY box in spr0119
atattctgtgaaaaataaaatagtat

SEQ ID NO: 173 - DNA, 26 bp, Artificial Sequence
  presence of the putative CodY box in hsdS 2X
gaatttccagataaactaaaaaaatc

SEQ ID NO: 174 - DNA, 26 bp, Artificial Sequence
  presence of the putative CodY box in spr0607
acattatctgaaaaattaaactataa

SEQ ID NO: 175 - DNA, 26 bp, Artificial Sequence
  presence of the putative CodY box in rpmI
taattgtcagaaagttaaataaagga

SEQ ID NO: 176 - DNA, 26 bp, Artificial Sequence
  presence of the putative CodY box in spnIM
aaattatctaataacaaaaatattat

SEQ ID NO: 177 - DNA, 26 bp, Artificial Sequence
  presence of the putative CodY box in spnIM
ttatttacagaaagaacaaaaaatgc

SEQ ID NO: 178 - DNA, 26 bp, Artificial Sequence
  presence of the putative CodY box in ilvD
gaattttcagaaaattctatacgcat

SEQ ID NO: 179 - DNA, 26 bp, Artificial Sequence
  presence of the putative CodY box in arcA
atattttttgaaaaattttttaaaaa

SEQ ID NO: 180 - DNA, 26 bp, Artificial Sequence
  presence of the putative CodY box in ABC-NBD
aaattgacaaataaaaatttgaatat

SEQ ID NO: 181 - DNA, 26 bp, Artificial Sequence
  presence of the putative CodY box in mfd
tactttacagaagaattacagaatag

SEQ ID NO: 182 - DNA, 26 bp, Artificial Sequence
  presence of the putative CodY box in spr0157
gaattttcagaataatctgtatatgt

SEQ ID NO: 183 - DNA, 26 bp, Artificial Sequence
  presence of the putative CodY box in murM
gaatttcctgaaaatctggctattat

SEQ ID NO: 184 - DNA, 26 bp, Artificial Sequence
  presence of the putative CodY box in zwf
gatttttcggaaaattatgttagaat

SEQ ID NO: 185 - DNA, 26 bp, Artificial Sequence
  presence of the putative CodY box in licD1
caatatttagaaaaaaaagaaattaa

SEQ ID NO: 186 - DNA, 26 bp, Artificial Sequence
  presence of the putative CodY box in spr1403
atattttctgaacaattaatatattc

SEQ ID NO: 187 - DNA, 26 bp, Artificial Sequence
  presence of the putative CodY box in relA
cattttgcggaaaattgagtaaatat

SEQ ID NO: 188 - DNA, 26 bp, Artificial Sequence
  presence of the putative CodY box in spr1650
taattttctgattttttgtaaaataa

SEQ ID NO: 189 - DNA, 26 bp, Artificial Sequence
  presence of the putative CodY box in pcpA
ttattttctaatagatataaaattat

SEQ ID NO: 190 - DNA, 26 bp, Artificial Sequence
  presence of the putative CodY box in rsuA
taaatttcagattgatgaaaaaatag

SEQ ID NO: 191 - DNA, 26 bp, Artificial Sequence
  presence of the putative CodY box in ptsG
aattttttagaaaaaagtgtaatttt

SEQ ID NO: 192 - DNA, 26 bp, Artificial Sequence
  presence of the putative CodY box in hsdS
aaattcactgaaagtttaaatatgac

SEQ ID NO: 193 - DNA, 26 bp, Artificial Sequence
  presence of the putative CodY box in spr0803
gaattgccagactattttaatactat

SEQ ID NO: 194 - DNA, 26 bp, Artificial Sequence
  presence of the putative CodY box in spr0826
agattttaagtaaaatttattagtaa

SEQ ID NO: 195 - DNA, 26 bp, Artificial Sequence
  presence of the putative CodY box in flaV
aaattttagaaaaaattaaagaatac

SEQ ID NO: 196 - DNA, 26 bp, Artificial Sequence
  presence of the putative CodY box in ABC-NP
caattatctgatcatctgaaaaatat

SEQ ID NO: 197 - DNA, 15 bp, Artificial Sequence
  presence of the putative CodY box in gbs1632
attatcagaatattg

SEQ ID NO: 198 - DNA, 15 bp, Artificial Sequence
  presence of the putative CodY box in gbs1105
attttcagaaaaata

SEQ ID NO: 199 - DNA, 15 bp, Artificial Sequence
  presence of the putative CodY box in gbs0662
tttatcagaaaattt

SEQ ID NO: 200 - DNA, 15 bp, Artificial Sequence
  presence of the putative CodY box in gbs2002
attttctgaatattc

SEQ ID NO: 201 - DNA, 15 bp, Artificial Sequence
  presence of the putative CodY box in gbs1489
tttttctgaatattt

SEQ ID NO: 202 - DNA, 15 bp, Artificial Sequence
  presence of the putative CodY box in gbs0054
attttcaaaaaattt

SEQ ID NO: 203 - DNA, 15 bp, Artificial Sequence
  presence of the putative CodY box in gbs0008
attttcaaaaaattg

SEQ ID NO: 204 - DNA, 15 bp, Artificial Sequence
  presence of the putative CodY box in gbs1406
attgtcagaattttc

SEQ ID NO: 205 - DNA, 15 bp, Artificial Sequence
  presence of the putative CodY box in gbs0144
attatctgaaaattt

SEQ ID NO: 206 - DNA, 15 bp, Artificial Sequence
  presence of the putative CodY box in gbs0898
attatctgaatatta

SEQ ID NO: 207 - DNA, 15 bp, Artificial Sequence
  presence of the putative CodY box in gbs2007
attttcagtatattc

SEQ ID NO: 208 - DNA, 15 bp, Artificial Sequence
  presence of the putative CodY box in gbs0235
attttcagaaaatgt

SEQ ID NO: 209 - DNA, 15 bp, Artificial Sequence
  presence of the putative CodY box in gbs0577
attatcagaagattt

SEQ ID NO: 210 - DNA, 15 bp, Artificial Sequence
  presence of the putative CodY box in gbs0143
attttcagataattg

SEQ ID NO: 211 - DNA, 15 bp, Artificial Sequence
  presence of the putative CodY box in gbs0604
atttttagaaaatta

SEQ ID NO: 212 - DNA, 15 bp, Artificial Sequence
  presence of the putative CodY box in dnaG/ ftsA
attttctgaataatt

SEQ ID NO: 213 - DNA, 15 bp, Artificial Sequence
  presence of the putative CodY box in gbs1259
attttcaggatattt

SEQ ID NO: 214 - DNA, 288 bp, Lactobacillus lactis
  Xaa (243)..(288): CDS
  Xaa (234)..(237): RBS
  Xaa (198)..(203): -10_signal
  Xaa (175)..(179): -35_signal
ctgatatcaataaagaataaaaagtccaaaacagagcagcatgaaaaataaaacaataataaaagcagtttttagtatgattactgcttttattatttcctccaaaacttttgctttacctttatttcgcgtaatgttcagaaaattcatgaacatacctaaaatagtaaatttttgcaaatatgcagaaaaagtagtatacttttattaagtctatttagaaagattttattgaggtaaatatggaaagtgaaaatattttggaagcaaaacaagtgagtgttgctt

SEQ ID NO: 215 - protein, 15 aa, Lactobacillus lactis
MESENILEAKQVSVA

SEQ ID NO: 216 - DNA, 26 bp, Artificial Sequence
  WT
aatgttcagaaaattcatgaacatac

SEQ ID NO: 217 - DNA, 26 bp, Artificial Sequence
  Opp15 (a)
aatattaagaaaattcatgaacatac

SEQ ID NO: 218 - DNA, 26 bp, Artificial Sequence
  Opp15 (b)
actgtgccgaaaattcatgaacatac

SEQ ID NO: 219 - DNA, 26 bp, Artificial Sequence
  Opp 2
aactgcaggaaaattcatgaacatac

SEQ ID NO: 220 - DNA, 30 bp, Artificial Sequence
  WT
cgtaatgttcagaaaattcatgaacatacc

SEQ ID NO: 221 - DNA, 30 bp, Artificial Sequence
  MUT2
cgtaatgttctgaaaattcatgaacatacc

SEQ ID NO: 222 - DNA, 30 bp, Artificial Sequence
  MUTF
cgtaatgttcagaaaattcatggacatacc

SEQ ID NO: 223 - DNA, 30 bp, Artificial Sequence
  MUT4
cgtaatgttcagaaaattcatgagcatacc

SEQ ID NO: 224 - DNA, 30 bp, Artificial Sequence
  MUT3
cgtagtgttcagaaaattcatgaacatacc

SEQ ID NO: 225 - DNA, 30 bp, Artificial Sequence
  MUT10
cgtaatgtccagaaaattcatgaacatacc

SEQ ID NO: 226 - DNA, 30 bp, Artificial Sequence
  MUT16
cgtaatgttcggaaaattcatgaacacacc

SEQ ID NO: 227 - DNA, 26 bp, Artificial Sequence
  motif as observed in L. lactis
  n (1)..(26): /note=“Sequence wherein n can be any nucleotide”
anaattttctganaaatnnatnanta

SEQ ID NO: 228 - DNA, 26 bp, Artificial Sequence
  consensus motif as observed in L. lactis
  n (1)..(26): /note=“Sequence wherein n can be any nucleotide”
whaattdtcwgahaawtnnrwnadww

SEQ ID NO: 229 - DNA, 15 bp, Artificial Sequence
  consensus motif as observed in B. subtilis
awttdtcagaawwwt

SEQ ID NO: 230 - DNA, 14 bp, Artificial Sequence
  motif as observed in B. subtilis
  n (1)..(14): /note=“Sequence wherein n can be any nucleotide”
attntcagaaaatt

SEQ ID NO: 231 - DNA, 225 bp, Lactococcus lactis Wg2
  promoter (1)..(225): /note=“prtP/prtM promoter region”
tgctaaaaatttcaaaacatctatagtctgtaaacggctaaataataacgctaaaagttaatttacagataaaaaaattaatagaagattaaaattttcgttgaatttgttcttcaatagtatataatataatagtatataatattatataatataatcttaactacatcaagcgtagggtttgatttggttatgaaacttttggaaagtggaggatattggatg

SEQ ID NO: 232 - DNA, 230 bp, Lactococcus lactis SK11
  promoter (1)..(230): /note=“prtP/prtM promoter region”
tgctaaaaatttcaaaacatctatagtctgtaaacggctaaataataacgctaaaagttaatttacagataaaaaaattaatagaagattaaaattttcgttgaatttgttcttcaatagtatataatataatagtatataatattatattatataatataatcttaactacatcaagcgtaggctttgatttggttatgaaacttttggaaagtggaggatattggatg

SEQ ID NO: 233 - DNA, 230 bp, Lactococcus lactis E8
  promoter (1)..(230): /note=“prtP/prtM promoter region”
tgctaaaaatttcaaaacatctatagtctgtaaacggctaaataataacgctaaaagttaatttacagataaaaaaattaatagaagattaaaattttcgttgaatttattcttcaatagtatataatataatattatataatattatattatataatataatcttaactacatcaagcgtagggtttgatttggttatgaaacttttggaaagtggaggatattggatg

SEQ ID NO: 234 - DNA, 230 bp, Lactococcus lactis BGMN1-5
  promoter (1)..(230): /note=“prtP/prtM promoter region”
tgctaaaaatttcaaaacatctatagtctgtaaacggctaaataataacgctaaaagttaatttacagataaaaaaattaatagaagattaaaattttagttgaatttgttctttaatagtatataatataatagtatatactattatattatatactattatattaactacatcaagcgtacattttgatttggttatgaaacttttggaaagtggagggtattggatg

(19) United States
(12) Patent Application Publication
(10) Pub. No.: US 2007/0243303 A1
(43) Pub. Date: Oct. 18, 2007

(54) Methods and Means for Regulating Gene Expression

(75) Inventors: Christiaan Daniel Dan Hengst, Groningen (NL); Olivera Gajic, Bennekom (NL); Oscar Paul Kuipers, Groningen (NL); Jan Kok, Groningen (NL); Jan Sikkema, Zeegse (NL); Johannes Marie Wilhelmus Geurts, Hengelo (NL); Arjen Nauta, Bennekom (NL)

Correspondence Address: NORRIS, MCLAUGHLIN & MARCUS, 875 THIRD AVE, 18TH FLOOR, NEW YORK, NY, 10022, US

(73) Assignee: Friesland Brands B.V., Meppel (NL)

(21) Appl. No.: 10/562,601

(22) PCT Filed: Jul. 2, 2004
(86) PCT No.: PCT/NL04/00474

§ 371 Date: Mar. 1, 2007

(30) Foreign Application Priority Data
Jul. 2, 2003 (EP) 03077074.7

(51) Int. Cl. A23C 19/00; A23C 9/00; C07H 21/00; C12N 1/21; C12N 15/00; C12N 15/74; C12P 21/00

(52) U.S. Cl. 426/582; 426/580; 435/252.3; 435/320.1; 435/471; 435/69.1; 536/23.2; 536/23.7

(57) ABSTRACT

The invention relates to methods and means for regulating gene expression and to CodY target sequences. CodY represses its target genes by binding to specific DNA sequences upstream of the respective genes. A conserved target site was identified by analyzing upstream sequences of derepressed genes in a delta codY L. lactis MG1363 derivative, as identified in a DNA micro-array study. Also, CodY target sequences from other gram-positive bacteria, like B. subtilis and Streptococcus are disclosed. Hence, the invention provides CodY target sequences that may be used in different applications to repress or derepress gene expression.

The invention relates to the field of biochemistry, molecular biology and food production. More in particular, the invention relates to methods and means for regulating gene expression. Even more in particular, the invention relates to CodY target sequences.

The Gram-positive lactic acid bacterium Lactococcus lactis is an important microorganism in dairy food production. It is part of many starter cultures used in cheese manufacturing, where its function is to degrade the milk protein casein into small peptides and amino acids (Kok and Vos, 1993). L. lactis, like other lactic acid bacteria, is a multiple amino acid auxotroph. It has a complex proteolytic system to break down the major milk protein casein into small peptides and free amino acids that are necessary for growth in this medium (Kunji et al., 1996, Christensen et al., 1999). Initial breakdown of casein is carried out by the extracellular cell wall-bound serine proteinase PrtP. Several lactococcal prtP genes have been cloned and sequenced (Kok et al., 1985, Kok et al., 1988, de Vos et al., 1989, Kiwaki et al., 1989). Although they are over 98% identical on the amino. acid sequence level, the proteinases can have quite different caseinolytic specificities (Visser et al., 1986). For the production of an active proteinase, the product of prtM, a gene that is in a back-to-back orientation with prtP, is required. The so-called maturase PrtM plays a role as an extracellular chaperone, inducing the pro-proteinase to adopt a conformation in which it is able to autoproteolytically cleave off its pro-region (Kok, 1990, Haandrikman, 1990). Peptides that are produced by the proteinase can be internalized by either one of three different transport systems. Oligopeptides are taken up by Opp, while DtpT and DtpP transport di- and three-peptides respectively (Tynkkynen et al., 1993, Foucaud et al., 1995). Intracellularly, the peptides are further hydrolyzed into smaller peptides and amino acids by the action of over 15 different peptidases (Kunji et al., 1996, Christensen et al., 1999).

Proteinase and maturase production is inhibited in peptide-rich medium (e.g. containing casitone, a tryptic digest of casein) in a number of lactococcal strains (Exterkate, 1985, Laan et al., 1993, Marugg et al., 1995, Miladinov et al., 2001). As PrtP expression is not down-regulated in strains that lack the di- and tripeptide transporter DtpT, it was hypothesized that the internal concentration of small (di-tri) peptides, or amino acids derived thereof, are important in the regulation of proteinase production (Marugg et al., 1995). The genetic information for proteinase regulation was shown to be present on a 90-bp subfragment of the prtP/prtM intergenic region encompassing the transcription start sites of both genes (Marugg et al., 1996). Disruption of an inverted repeat that is present in this region resulted in derepression of the prtP and prtM promoters in medium with a high peptide concentration.

The expression of genes of other components of the proteolytic system of L. lactis is also affected by medium composition. The expression of OppA, DtpT and DtpP is increased when cells are grown in medium with a low peptide concentration (Detmers et al., 1998, Kunji et al., 1996, Foucaud et al., 1995). Moreover, the expression of the peptidases PepX and PepN in L. lactis MG1363 was shown to be regulated in a similar way (Meijer et al., 1996). Promoters of pepC, pepN, pepO1, and pepO2 were also reported to be more active in medium with amino acids than in peptide-rich medium (Guedon et al., 2001a). In the same study, the prtP promoter was shown to be subject of a similar regulatory circuit.

Recently, a pleiotropic regulator, CodY, has been identified in L. lactis MG1363 that represses several genes involved in the processes mentioned above (Guedon et al., 2001b). CodY, was first identified in the Gram-positive bacterium Bacillus subtilis, in which it also serves as a repressor of several genes involved in proteolysis (Serror and Sonenshein, 1996b; Serror and Sonenshein, 1996a). In B. subtilis, the activity of CodY is dependent on intracellular GTP levels, thereby sensing the energy state of the cell (Ratnayake-Lecamwasam et al., 2001). For L. lactis it was shown that the repression by CodY is relieved upon a decrease in the intracellular pool of the branched chain amino acids (BCAA's) Leu, Iso and Val (Guedon et al., 2001b).

Like its B. subtilis counterpart, CodY of L. lactis contains a C-terminal helix-turn-helix DNA binding motif. In B. subtilis it has been shown that the protein is able to bind to sequences overlapping the −35 and −10 sequences of its target promoters (Serror and Sonenshein, 1996b; Fisher, Rohrer, and Ferson, 1996).

Herein we show that CodY represses its target genes by binding to specific DNA sequences upstream of the respective genes. A conserved target site was identified by analyzing upstream sequences of derepressed genes in a delta codY L. lactis MG1363 derivative, as identified in a DNA micro-array study. The present application furthermore discloses CodY target sequences from other gram-positive bacteria, like B. subtilis and Streptococcus.

Hence, the invention provides CodY target sequences that may be used in different applications to repress or derepress gene expression.

In a first embodiment, the invention provides a method for regulating the expression of a gene of interest in a host cell that comprises a CodY-like protein comprising providing said cell with a gene of interest in operable linkage with a promoter and at least one CodY target sequence.

Regulation of gene expression is a very desirable characteristic of gene expression systems. For example, when one would like to express a protein that is toxic for the used host cell, preferably a gene encoding said protein is under the control of a regulator which can be switched on or off at will. Typically, for production of such a protein, expression of the corresponding gene is suppressed until enough biomass has been obtained and then expression of said gene is obtained for example by providing an inducer. However, also expression of non-toxic proteins is preferably regulated by an induction system. Examples of these kinds of expression systems are well known in the art and hence no further elaboration on this subject is necessary. In a method according to the invention a CodY-like protein and at least one CodY target sequence, control expression of a gene of interest. Binding of a CodY-like protein to said at least one CodY target sequence results in repression of expression of the gene of interest that is under control of said CodY target sequence. In the absence of (sufficient) CodY-like protein or in the presence of non-functional (i.e. non-binding) CodY-like protein, said gene of interest is expressed. Hence, the invention provides a way for regulating gene expression. As CodY-like proteins are typically found in gram-positive bacteria, for example lactic acid bacteria, the invention preferably provides a method for regulation gene expression in gram-positive bacteria. However, it is clear to the person skilled in the art that necessary components of the method according to the invention, i.e. a CodY-like protein and a CodY target sequence may easily be transferred to for example a gram-negative bacterium.

CodY proteins show a large amount of homology in gram-positive bacteria with a low G+C content. A Blast search with the CodY amino acid sequence of L. lactis shows homology with Bacillus subtilis, Bacillus anthracis, Bacillus halodurans, Bacillus stearothermophilus, Clostridium acetobutylicum, Clostridium difficile, Enterococcus faecalis, Staphylococcus aureus, Streptococcus mutans, Streptococcus pneumoniae and Streptococcus pyogenes. Moreover, these CodY proteins all comprise a DNA binding motif, preferably a helix-turn-helix DNA binding motif. It is furthermore shown that CodY proteins comprise putative GTP binding motifs as summarised in Table 1. Furthermore, binding of these CodY proteins to their target sequences is typically under the influence of the energy level of the cell (GTP) or the intracellular pool of branched chain amino acids or the nutritional value of the medium (nitrogen source like casitone).

A CodY-like protein is typically a CodY protein or a functional equivalent and/or a functional fragment thereof, obtained/derived from a gram-positive bacterium, which CodY protein comprises the above outlined characteristics, i.e. capable of binding to a (consensus) CodY target sequence and sensitive to a change in the energy level of the cell, the intracellular pool of branched amino acids or the medium composition. Examples of CodY proteins are the CodY proteins from Lactococcus lactis (Guedon et al, 2001b)) or Bacillus subtilis (Serror and Sonenshein, 1996b; Serror and Sonenshein, 1996a). It is clear that for example a CodY protein from L. lactis can be modified without significantly changing the above outlined characteristics, for example, by introducing point mutations or (small) deletions. Hence, a CodY-like protein is a Cod protein obtained from a gram-positive bacterium such as Bacillus subtilis, Bacillus anthracis, Bacillus halodurans, Bacillus stearothermophilus, Clostridium acetobutylicum, Clostridium difficile, Enterococcus faecalis, Staphylococcus aureus, Streptococcus mutans, Streptococcus pneumoniae, Streptococcus pyogenes, and Lactococcus lactis, possibly comprising mutations which do not interfere significantly with for example the binding of said CodY-like protein to a CodY target sequence and furthermore is sensitive to the energy state of a cell, the intracellular pool of branched amino acids or the medium composition.

The location of the CodY target sequence with regard to the promoter sequence is flexible. The CodY target sequence may be either located upstream, downstream or overlapping with regard to the −35 and −10 sequences. Furthermore, at least one CodY target sequence is used in a method according to the invention. As disclosed herein within the experimental part, an increase in the number of CodY target sequences results in a more pronounced regulation of expression and hence introduction of more than one CodY target sequence is useful depending on, for example, the characteristics of the gene of interest.

The promoter used in a method according to the invention is preferably a promoter that is functional in the used host cell. For example, a promoter that is functional in a gram-positive bacterium is used, in operable linkage with a CodY target sequence and a gene of interest, for regulating expression of said gene of interest in a gram-positive bacterium. The prior art provides a large amount of promoter sequences, both from gram-positive as well as gram-negative bacteria, and hence no further elaboration on this item is necessary.

In a preferred embodiment, said promoter and/or said CodY target sequence is heterologous with regard to said gene of interest. In yet another preferred embodiment, said CodY target sequence is heterologous with regard to said promoter. Hence, the invention preferably makes use of combinations in which at least one component (i.e. promoter sequence or gene of interest or CodY target sequence) is different when compared to wild type/natural situation. The gene of interest may be an endogenous gene or a heterologous gene. For an endogenous gene that is already in operable linkage with a promoter, only at least one CodY target sequence has to be introduced (in operable linkage with said promoter and said gene). After introduction of said at least one CodY target sequence, expression of said endogenous gene will, in the presence/absence of CodY-like protein, be repressed/derepressed and hence expression of said endogenous gene is regulated. Furthermore, it is within the scope of the present application to introduce an extra copy of an endogenous gene in operable linkage with its own promoter and/or at least one CodY target sequence or with another promoter and/or at least one CodY target sequence. Hence, at least two copies of said endogenous gene are then present. A heterologous gene in operable linkage with a promoter and at least one CodY sequence may for example be introduced via a plasmid. However, it also possible to only introduce said gene of interest and further provide said gene of interest with the necessary means for homologous recombination to an endogenous gene that is under control of a CodY target sequence. In this way an endogenous gene is replaced by another gene, which is, then under control of a CodY target sequence.

The introduction of new and/or extra genetic information into a host cell may be accomplished by methods known in the art, for example by electroporation, protoplast transformation, transfection, transduction or any other known method.

Any sequence can be used as sequence of interest. Preferably, said sequence enables the production of a protein of interest not present as such or present in a (too) low concentration, in said cell. For example, a sequence/open reading frame (ORF) specifying an enzyme (protease or peptidase), a vitamin or an anti-microbial peptide is used. Preferably, said gene of interest is a gene from a gram-positive bacterium. Even more preferably, said gene of interest is a gene from a lactic acid bacterium, like Lactococcus, Lactobacillus, Streptococcus, Leuconostoc, Pediococcus, Bifidobacterium, Carnobacterium or Propionibacterium. An example of a gene of interest is a gene that encodes a protease or a peptidase or an anti-microbial peptide or a vitamin. Other examples of gene products include, but is not limited to, hydrolytic enzymes selected from proteases such as chymosin, peptidases including endopeptidases, lipases, nucleases and carbohydrases; lytic enzymes such as lysozyme or phage lysins; flavour enhacing substances; bacteriocins including nisin, pediocin and bavaracin; amino acids; organic acids; and pharmacologically active substances. Further examples comprise genes of which the products make host cells more resistant to advere conditions, for instance conditions to which micro-organisms are confronted at various stages during industrial use, e.g. starvation, lactic acid accumulation, oxygen stress, drying-stress, temperature stress. For example probiotic formula's are made more robust during and/or after production, e.g. by optimising the survival of cells of probiotic strains. As another example, the re-growth of starter cultures is made more reliable.

In a preferred embodiment the invention provides a method for regulating the expression of a gene of interest in a host cell that comprises a CodY-like protein comprising providing said cell with a gene of interest in operable linkage with a promoter and at least one CodY target sequence, wherein said CodY target sequence comprises a sequence as depicted in the upper part of FIG. 6A, or a functional equivalent and/or a functional fragment thereof. The upper part of FIG. 6A discloses a consensus CodY target sequence. In another preferred embodiment the invention provides a method for regulating the expression of a gene of interest in a host cell that comprises a CodY-like protein comprising providing said cell with a gene of interest in operable linkage with a promoter and at least one CodY target sequence, wherein said CodY target sequence comprises a sequence as depicted in FIG. 6B, or a functional equivalent and/or a functional fragment thereof. Moreover, the invention provides in Table 4 and Table 4A multiple examples of L. lactis CodY target sequences that provide non-limiting examples of combinations of W, R, D and H as depicted in FIGS. 6A and 6B. Until the present patent application, no (consensus) sequence for CodY binding was disclosed. Now that the consensus sequence and some of its variants are disclosed herein (see upper part of FIG. 6A, Table 4 and Table 4A) a person skilled in the art is very well capable of obtaining a functional equivalent and/or a functional fragment of said consensus sequence. A functional equivalent and/or a functional fragment must still be capable of binding a CodY-like protein. A functional equivalent is for example obtained by screening other bacteria for the presence of the herein disclosed CodY target sequences. For example, the present inventors have identified CodY target sequences in Bacillus subtilis, Streptococcus pneumoniae and Streptococcus agalacticiae, as disclosed herein within FIG. 6A lower part, Table 5, 6, 7 or 8. The lower part of FIG. 6A discloses the CodY target consensus sequence in B. subtilis and Table 5 and 6 show multiple examples of the typical CodY target sequences. Table 7 and 8 disclose multiple examples of Streptococus CodY target sequences. It is clear that point mutation and deletion studies lead to further functional equivalents and/or functional fragments and hence these also within the scope of the present patent application.

One embodiment of the present invention provides a method for regulating the expression of a gene of interest in a host cell that comprises a CodY-like protein comprising providing said cell with a gene of interest in operable linkage with a promoter and at least one CodY target sequence, wherein said CodY target sequence comprises an ATGTTCA sequence and an inversely repeated ATGTTCA sequence. An example of such sequence is shown in FIG. 1. As is shown in the examples, said CodY target sequence is involved in CodY-mediated regulation of PoppD. Preferably, said nucleic acid sequence comprises a spacing of about 9 base pairs between said ATGTTCA sequence and said inversely repeated ATGTTCA sequence. More preferably, said nucleic acid sequence comprises the sequence ATGTTCAGAAAATTCATGAACAT.

Based on the herein disclosed (consensus) CodY target sequences it is furthermore possible to construct for example constructs comprising two or more (identical or different) CodY-like target sequences. In this way a more stringent regulation of expression is obtained. For example a gene of interest in operable linkage with a promoter and two (identical or different) CodY target sequence is used to obtain more stringent control of expression. However, it is also possible to introduce a construct that comprises multiple CodY target sequences (with or without a promoter and/or a gene of interest) in a cell that comprises CodY regulated genes and hence a competitive binding of CodY to said construct that comprises multiple CodY target sequences and binding to a CodY target sequence in operable linkage with a gene of interest and a promoter takes place. In this way a gene of interest is derepresses and said gene of interest is expressed.

The method according to the invention allows both active as well as inactive/passive regulation of gene expression of a gene of interest. Said regulation is preferably based on influencing the binding between a CodY-like protein and at least one CodY target sequence. An example of passive/indirect/inactive regulation is a gene of interest in operable linkage with a promoter and a CodY target sequence that is introduced into a host cell that comprises CodY-like protein. During exponential growth of said host cell said CodY-like protein binds to said CodY target sequence and hence expression of said gene of interest is repressed. After the exponential phase, said CodY-like protein will release from said CodY target sequence and hence expression of said gene of interest is induced. Such an approach is extremely useful in cases in which one would like to have expression of a gene of interest after exponential growth. Active regulation of gene expression of a gene in operable linkage with a promoter and a at least one CodY target sequence is for example obtained by regulating binding of a CodY-like protein and a CodY target sequence by subjecting said cell to a change in a growth condition, preferably to a growth limiting condition like a limited availability of a nitrogen source. In case, a CodY-like protein or a functional fragment and/or a functional derivative thereof of L. lactis is used, means that result in a decrease in the intracellular pool of the branched amino acids Leu, Iso and Val results in relief of CodY repression. The CodY protein of B. subtilis is for example actively regulated by a means that influence the level of GTP in a host cell. Hence, actively subjecting a CodY-like protein comprising host cell that further comprises a gene of interest in operable linkage with a promoter and at least one CodY target sequence to a medium with a limited availability of a nitrogen source results in derepression and hence expression of said gene of interest.

In a preferred embodiment, the invention provides a method for regulating the expression of a gene of interest in a host cell that comprises a CodY-like protein comprising providing said cell with a gene of interest in operable linkage with a promoter and at least one CodY target sequence, wherein said host cell is a cell from a food production species and even more preferably a dairy food production species. Preferably, said species is selected from the gram-positive species and even more preferably said species is a lactic acid bacterium such as Lactococcus or Lactobacillus or Streptococcus or Leuconostoc or Pediococcus or Bifidobacterium or Carnobacterium. An example of a gram-positive, non lactic acid bacterium is Propionibacterium. Amongst others, these species are used in the production of food, for example in a fermentation step for the production of a dairy product. Hence, by providing these species with a gene of interest under the control of a promoter and at least one CodY target sequence and either indirectly/passively or directly/actively influencing the binding between a CodY-like protein and its target sequence results in repression or derepression (i.e regulation) of gene expression of said gene of interest. This may be used for the metabolic engineering of various catabolic pathways by a rerouting strategy consisting of the controlled overproduction and/or disruption of genes. For example, genes of which the products, directly or indirectly, are involved in the production of compounds that are involved in the formation of off-flavours during exponential growth during a (dairy) food production, are repressed by providing said genes with a CodY target sequence. Food or dairy food production species in which said genes are under the control of a CodY target sequence, will produce less (or preferably no) off-flavours and hence these production processes are improved. It has for instance become possible to alter flavour formation in cheese, yoghurt and/or other fermented (dairy) products by altering the expression of enzymes that, when present in different quantities, give rise to re-routing of specific pathways. Examples of such enzymes are enzymes involved in e.g.
1) lactose, citrate and diacetyl metabolism, and alcohol metabolism,
2) lipid degradation, modification and synthesis,
3) polysaccharide synthesis,
4) amino acid degradation,
5) protein and carbohydrate utilisation and conversion,
6) cell lysis (e.g. bacteriophage and host cell-encoded cell wall hydrolases and DEAD-box helicase proteins) and cell wall synthesis.

In an analogous way it is also possible to induce expression of a gene of interest after the exponential growth and hence provide said species with altered flavour formation, altered cell lysis capabilities or induce production of antimicrobial substances and/or health promoting substances (such as vitamins) or provide said species with means to at least in part prevent acidification of the same or another species. For the latter possibilities, a gene of interest (for example a gene involved in cell lysis or flavour formation or a gene encoding a vitamin) is placed under the control of a promoter and at least one CodY target sequence and after the end of exponential growth, CodY-like protein will be released from said CodY target sequence and hence expression of said gene is induced. Furthermore, CodY-like proteins are released from their target sequences by providing cells with for example synthetic CodY targets. Said CodY target may be added to the medium, taken up by the cells (for example B. subtilis) and the CodY-like proteins are released from their targets and will bind to the synthetic CodY targets. With a method of the invention it has for instance become possible to induce the production of antimicrobial substances (for instance antimicrobial peptides) of which the production would be detrimental during the fermentation process but is benificial after exponential growth to prevent spoilage organisms in the fermented product. A method of the invention is for instance suitable for inducing bacteriocin (e.g. nisin) production in cheese, yoghurt and/or other fermented (dairy) products after exponential growth thereby preventing that the fermenting micro-organisms are affected during fermentation.

In another aspect a method of the invention is used in order to decrease the expression of a gene in a stationary phase culture or equivalent of said culture. This is for instance done by providing a host cell with an antisense nucleic acid sequence in operable linkage with a promoter and at least one codY target sequence. In the presence of CodY, expression of said antisense nucleic acid is repressed during exponential growth. Upon transcription of said antisense nucleic acid during the stationary phase, produced RNA will bind mRNA of said undesired gene, thereby preventing translation of said undesired gene. Said undesired gene for instance comprises a gene involved in post-acidification. Said gene for instance comprises a gene involved in carbon catabolism, glucose and lactose catabolism (such as for instance glycolytic enzymes/lactate dehydrogenase) and lactose uptake. It has also become possible to prevent undesired CO₂ production by inhibiting citrate catabolism, to prevent off-flavour production or re-routing of specific pathways by inhibiting expression of a gene encoding undesired proteins/peptides, and/or to extend the shelf life of fermented (dairy) products by decreasing metabolism, ((post-)acidification, glycolysis, lipolysis, proteolysis, peptidolysis) and/or cell lysis.

Hence, with a method of the present invention expression of a gene can be regulated at any stage of growth, for instance during exponential growth and/or during the stationary phase. In a further aspect a method of the invention is used in order to decrease expression of a gene, wherein binding of a CodY-like protein to a CodY target sequence is regulated by subjecting said cell to a change in a growth condition. Preferably, said cell is subjected to a growth limiting condition. In a preferred embodiment said growth limiting condition is a limited availability of a nitrogen source.

Now that the invention discloses multiple (consensus) CodY target sequences it is furthermore part of the invention to modify endogenous CodY sequences, to for example increase or decrease (or more generally alter) binding of CodY to one of its endogenous target sequences and hence production of an endogenous gene that is under control of the CodY/CodY target sequence regulation may be altered. For example, proteins which expression is under the control of CodY (and hence are not or hardly not produced during the exponential growth phase) but whose product provides advantageous uses when present during exponential growth may now be amended such that binding of CodY to said CodY target sequence is not or hardly not possible under the exponential growth phase conditions. Hence, said protein is expressed during exponential growth and advantage is taken of the properties of said expressed protein.

In case a host cell does not comprise or does not comprise enough CodY-like protein or comprises non-functional (i.e. CodY protein that is not capable of binding to a CodY target sequence) CodY-like protein, the invention furthermore provides a method for regulating the expression of a gene of interest in a host cell that comprises a CodY-like protein comprising providing said cell with a gene of interest in operable linkage with a promoter and at least one CodY target sequence wherein said host cell is further provided with a nucleic acid encoding a CodY-like protein. Examples of CodY sequences are already outlined above and hence no further details are provided. In case a host cell does not comprise/express enough CodY-like protein said cell may be provided with either endogenous and/or heterologous CodY-like protein.

In yet another preferred embodiment, the invention provides an isolated or recombinant nucleic acid that comprises at least one CodY target sequence or a functional fragment and/or a functional equivalent thereof. It is clear from FIG. 6A (lower and upper part), FIG. 6B and Tables 4 to 8, that the length of said nucleic acid generally comprises about 15 to 30 nucleotides. A functional fragment and/or a functional equivalent thereof is defined as a fragment and/or equivalent that is capable of binding a CodY-like protein. A functional fragment is for example obtained by introducing an N-, C- or internal deletion. Examples of suitable CodY target sequences are provided herein (FIG. 6A, FIG. 6B and Tables 4 to 8). A nucleic acid that comprises multiple (either identical or different) CodY target sequences is also included herein. Furthermore, artificial or synthetic CodY target sequences are also included herein.

In a preferred embodiment said isolated or recombinant nucleic acid that comprises at least one CodY target sequence or a functional fragment and/or a functional equivalent thereof, further comprises a promoter sequence and/or a promoter sequence in operable linkage with a gene of interest.

In case a host cell does not comprise or does not comprise enough endogenous CodY-like or comprises non-functional CodY protein, the invention furthermore provides an isolated or recombinant nucleic acid that comprises at least one CodY target sequence or a functional fragment and/or a functional equivalent thereof wherein said nucleic acid further comprises a gene encoding a CodY-like protein or a functional fragment and/or a functional equivalent thereof.

In a preferred embodiment, said promoter and/or said at least one CodY target sequence is heterologous with regard to said gene of interest and in another preferred embodiment, said CodY target sequence is heterologous with regard to said promoter.

Again, as already outlined above, said gene of interest may either be an endogenous and/or a heterologous gene. Preferably, said gene of interest is a gene from a gram-positive bacterium, such as a gene from a lactic acid bacterium for example Lactococcus or Lactobacillus or Streptococcus or Leuconostoc or Pediococcus or Bifidobacterium or Carnobacterium. An example of a gram-positive, non lactic acid bacterium is Propionibacterium.

A gene of interest may be any gene, preferably said gene of interest encodes a protease or a peptidase or an anti-microbial peptide or a vitamin. Other suitable examples include hydrolytic enzymes selected from proteases such as chymosin, peptidases including endopeptidases, lipases, nucleases and carbohydrases; lytic enzymes such as lysozyme or phage lysins; flavour enhacing substances; bacteriocins including nisin, pediocin and bavaracin; amino acids; organic acids; and pharmacologically active substances.

In a preferred embodiment, the invention provides an isolated or recombinant nucleic acid that comprises at least one CodY target sequence or a functional fragment and/or a functional equivalent thereof, wherein said CodY target sequence comprises a sequence as depicted in FIG. 6A and/or FIG. 6B, or a functional equivalent and/or a functional fragment thereof. The upper part of FIG. 6A, and FIG. 6B disclose a consensus CodY target sequence. Moreover, the invention provides in Table 4 and Table 4A multiple examples of L. lactis CodY target sequences that provide non-limiting examples of combinations of W, R, D and H as depicted in FIG. 6A and FIG. 6B. Until the present patent application, no (consensus) sequence for CodY binding was disclosed.

In a further preferred embodiment, the invention provides an isolated or recombinant nucleic acid that comprises at least one CodY target sequence or a functional fragment and/or a functional equivalent thereof, wherein said CodY target sequence comprises a sequence as depicted in Table 4, Table 4A, Table 5, Table 6, Table 7 and/or Table 8, or a functional equivalent and/or a functional fragment thereof.

In another preferred embodiment the invention provides an isolated or recombinant nucleic acid that comprises at least one CodY target sequence or a functional fragment and/or a functional equivalent thereof, wherein said CodY target sequence comprises an ATGTTCA sequence and an inversely repeated ATGTTCA sequence. Preferably, said nucleic acid sequence comprises a spacing of about 9 base pairs between said ATGTTCA sequence and said inversely repeated ATGTTCA sequence. More preferably, said nucleic acid sequence comprises the sequence ATGTTCAGAAAATTCATGAACAT.

Now that a consensus sequence and some of its variants are disclosed herein (see FIG. 6B, upper part of FIG. 6A, Table 4 and Table 4A) a person skilled in the art is very well capable of obtaining a functional equivalent and/or a functional fragment of said consensus sequence. A functional equivalent and/or a functional fragment must still be capable of binding a CodY-like protein. A functional equivalent is for example obtained by screening other bacteria for the presence of the herein disclosed CodY target sequences. For example, the present inventors have identified CodY target sequences in Bacillus subtilis, Streptococcus pneumoniae and Streptococcus agalacticiae, as disclosed herein within FIG. 6A lower part, Table 5, 6, 7 or 8. The lower part of FIG. 6A discloses the CodY target consensus sequence in B. subtilis and Table 5 and 6 show multiple examples of the typical CodY target sequences. Table 7 and 8 disclose multiple examples of Streptococus CodY target sequences. It is clear that point mutation and deletion studies lead to further functional equivalents and/or functional fragments and hence these also within the scope of the present patent application.

Moreover, the present inventors have identified alternative sequences in the upstream region of CodY regulated genes that may also be included in a method for regulating the expression of a gene of interest in a host cell that comprises a CodY-like protein (FIG. 7). These sequences may also be used in a method and/or nucleic acid according to the invention.

In another embodiment the invention provides a vector comprising a nucleic acid as described above. Said vector may further be provided with means for homologous recombination. With these means said at least one CodY target sequence and/or a gene of interest and/or a promoter and/or a gene encoding CodY-like protein may be integrated into the genome of a cell and hence a more stable situation may be obtained. In yet another embodiment, the invention provides a gene delivery vehicle comprising a nucleic acid or a vector according to the invention. Gene delivery vehicles are well known in the art and hence no further details are provided on this subject matter.

In a further embodiment the invention provides a host cell that comprises a nucleic acid, a vector or a gene delivery vehicle according to the invention. Preferably, said host cell is a cell from a food production species and even more preferably said host cell is a cell from a dairy food production species. Non-limiting examples of said species are gram positive lactic acid bacteria such as Lactococcus or Lactobacillus or Streptococcus or Leuconostoc or Pediococcus or Bifidobacterium or Carnobacterium. An example of a gram positive, non lactic acid species is Propionibacterium.

In another embodiment, the invention provides use of at least one CodY target sequence for regulating the expression of a gene of interest. Preferably, said at least one CodY target sequence is selected from FIG. 6A. FIG. 6B or Tables 4 to 8. For example, the use of at least one CodY target sequence in operable linkage with a promoter and a gene of interest in a Cod-like protein comprising host cell result, for example under exponential growth in repression of expression of said gene of interest. After exponential growth, CodY protein will be released from its target, resulting in depression of expression of said gene of interest.

In yet another embodiment the invention provides a method for producing a food product comprising a nucleic acid or a vector or a gene delivery vehicle or a host cell as described above. Preferably said food product is a dairy food product. As a non-limiting example, the use of a host cell is described in more detail. A lot of (dairy) food production processes involve the use of a (fermenting) host cell. These host cells may now be manipulated with regard their protein products. For example, genes of which the products, directly or indirectly, are involved in the production of compounds that are involved in the formation of off-flavours during exponential growth during a (dairy) food production, are repressed by providing said genes with a CodY target sequence. Food or dairy food production species in which said genes are under the control of a CodY target sequence, will produce less (or preferably no (detectable)) off-flavours during exponential growth and hence these production processes are altered. In an analogous way it is also possible to induce expression of a gene of interest after the exponential growth of said host cell and hence provide said species with altered flavour formation, altered cell lysis capabilities or induce production of antimicrobial substances and/or health promoting substances (such as vitamins) or provide said species with means to prevent acidification of the same or another species. For the latter possibilities, a gene of interest (for example a gene involved in cell lysis or a gene involved in flavour formation or a gene encoding a vitamin) is placed under the control of a promoter and at least one CodY target sequence and after the end of exponential growth, CodY-like protein will be released from said CodY target sequence and hence expression of said gene is induced.

Furthermore expression of undesired genes during the stationary phase can be, at least in part, decreased.

With regard to a fluid dairy product a method to at least in part decrease lysis of bacteria and/or acidification after production of said fluid diary product, i.e. after exponential growth of the used bacteria, is very advantageous with regard to the shelf life of said fluid dairy product. First, genes that are capable of at least in part preventing lysis and/or acidification are identified. After identification, such genes are placed under the regulation of at least one CodY target sequence. Said genes are expressed and hence lysis and/or acidification is at least in part prevented (and more preferably completely inhibited) and hence the shelf life of said product is increased.

Moreover, in case integration of said nucleic acid or vector is desired, use may be made of food grade integration techniques (for example see Leenhouts, 1995, herein incorporated by reference).

Preferably, the invention provides a method for producing a (dairy) food product comprising a nucleic acid or a vector or a gene delivery vehicle or a host cell as described herein, wherein said dairy product is a cheese or a fermented milk product. The production of a lot of dairy products involves fermentation of lactic acid bacteria and hence the application of modified host cells as described herein are particularly advantageous. The term “dairy product” include but is not limited to cheese, fluid dairy products like milk and yoghurt, fermented milk product, ice cream, butter, buttermilk, margarine and milk powder.

In another embodiment the invention provides food or a dairy food, such as a cheese or a fermented milk product, obtainable by a method according to the invention. Such a product comprises for example a different or extra taste or comprises specific compounds/structures (such as health improving compounds as vitamins) not present in food or dairy food product not obtained according to a method of the invention. Food or dairy food products prepared by a method according to the invention may comprise different amounts or different kinds of enzymes, peptides, amino acids, flavour enhancing or pharmacologically active substances or organic acids.

In yet another embodiment, the invention provides a method for at least in part preventing the formation of off-flavours during a process for the production of a (dairy) food product, comprising providing at least one CodY target sequence in operable linkage with a gene which product is, directly or indirectly, involved in the formation of off-flavours.

Now that the present inventors have disclosed CodY target sequences, interesting leads are provided in the fight against food-spoilers and pathogens.

PpmA is a recently identified pneumococcal protein with significant sequence homology to the proteinase maturation protein (PrtM) of lactic acid bacteria. PrtM is a trans-acting protein involved in the processing of precursors of serine protease PrtP into active enzymes and belongs to the family of peptidyl-prolyl cis/trans isomerases. These enzymes are thought to assist in protein folding by catalyzing the cis/trans isomerization of the petidyl-prolyl bonds in peptides and proteins. The pneumococcal proteins(s) that is activated by PpmA is currently unknown. PpmA of Streptococcus pneumoniae was demonstrated to be involved in virulence. Inactivation of ppmA significantly reduced the virulence of strain D39 for mice as judged by the survival time after intranasal challenge. The present inventors identified a CodY target sequence upstream of ppmA (Table 7) indicating that the expression of this gene probably is under the control of CodY. Hence, the invention provides a method for regulating the expression of ppmA.

Spore-forming bacteria (e.g. Bacillus) can cause serious problems in industrial food fermentations as the spores can survive most processing conditions. In Bacillus subtilis it was shown that the target genes of CodY generally encode proteins useful to the cell in adapting to poor nutritional conditions, but also include several genes whose expression is critical to the acquisition of genetic competence and the initiation of sporulation. The present invention provides a method for influencing the expression of these genes.

In Clostridium difficile, the synthesis of two toxin proteins responsible for antibiotic-associated colitis and pseudomembranous colitis were shown to be dependent on a RNA polymerase sigma factor TxeR (Sonenshein, unpublished). Both TxeR and the toxin proteins were not synthesized in exponential phase cells, probably due to the action of CodY as it was shown to bind to the toxin regulatory region.

The invention will now be illustrated by means of the following, non-limiting examples.

### EXPERIMENTAL PART

### Materials and Methods

Bacterial Strains, Plasmids and Growth Conditions

The strains and plasmids used in this study are listed in Table 9. Escherichia coli was grown in TY medium at 37° C. with vigorous agitation or on TY medium solidified with 1.5% agar, containing 100 μg of erythromycin per ml when needed. 5-bromo-4-chloro-3-indolyl-β-D-galactopyranoside (X-gal) was used at a final concentration of 40 μg/ml. L. lactis was grown at 30° C. in M17 broth or on M17 medium solidified with 1.5% agar, supplemented with 0.5% glucose. When needed, erythromycin, chloramphenicol and X-gal were added at final concentrations of 5 μg, 5 μg and 80 μg per ml, respectively. Chemically defined medium (CDM) was prepared according to Poolman and Koning (1988).

DNA Preparation, Molecular Cloning and Transformation

Routine DNA manipulations were performed as described by Sambrook et al.(1989). Total chromosomal DNA from L. lactis MG1363 was extracted as described previously. Plasmid DNA was isolated by the alkaline lysis procedure described by Sambrook et al (1989). Minipreparations of plasmid DNA from E. coli and L. lactis were made using the High Pure Plasmid isolation Kit from Roche, with minor modifications for L. lactis. Restriction enzymes and T4 DNA ligase were purchased from Roche. PCR amplifications were carried out using Pwo DNA polymerase for cloning fragments and Taq DNA polymerase for checking DNA insert sizes in plasmids from transformants. Electrotransformation of E. coli and L. lactis were performed with a Bio-Rad Gene Pulser (Bio-Rad Laboratories, Richmond, Calif.).

RNA Preparation and Primer Extension

The opp transcript was subjected to primer extension analysis using the oligonucleotide sto14 (CTTGCCATGGAATCACCCG) essentially as described previously (Buist et al, 1997). In the reactions, 30 μg of total RNA that was isolated from L. lactis MG1363 cells as described (van Asseldonk et al, 1993) was used as template. A DNA sequence ladder was obtained using the T7 sequencing kit (Amersham Pharmacia Biotech, Little Chalfont, United Kingdom) according to the manufacturers' descriptions.

Cloning of oppD Promoter Fragments

In order to study the regulation of promoter strength upstream of the oppD a deletion analysis of the respective promoters was carried out. Combinations of oligonucleotides oppl (5′ GCTCTAGACACTCACTTGTTTTGCTTCC 3′), opp2 (5′ AACTGCAGGAAAATTCATGAACATACC 3′), opp1-opp3 (5′ AACTGCAGTAAAACAATAATAAAAGCAG 3′), opp1-opp4 (5′ AACTGCAGGATAATAAAATTTGGACTG 3′), opp1-opp14 (5′ AACTGCAGCGTAATGTTCAGAAAATTC 3′), opp1-opp15 (a) (5′ AACTGCAGCGTAATATTTAGAAAATTCATGAACATACC 3′) and opp1-opp15 (b) (5′ AACTGCAGCGTACTGTGCCGAAAATTCATGAACATACC 3′) were used to amplify chromosomal DNA from L. lactis MG1363 in order to obtain fragments encompassing the oppD promoter. The PCR products were digested with XbaI and PstI and transcriptionally fused upstream of the promoterless lacZ gene in the integration vector pORI13 (Sanders et al., 1998), also digested with the same enzymes. The resulting plasmids were called pORIopp2, pORIopp3, pORIopp4, pORIopp14, pORIopp15 (a) and pORIopp15 (b). All pORI constructions were preformed in E. coli EC101 which contains a chromosomal copy of the lactococcal repA gene needed for replication. They were then transformed into L. lactis LL108 and/or LL302 which contain multiple and single chromosomal copies of repA, respectively.

Random Mutagenesis of oppD Promoter Region

PCR fragments encompassing the oppD promoter region containing randomly introduced base pair substitutions were obtained essentially as described (Spee, de Vos, and Kuipers, 1993). Chromosomal DNA isolated from L. lactis MG1363 cultures were used as a template in the amplification step. Subsequently, the obtained variants were cloned into plasmid pORI13 and introduced into L. lactis LL108 as described above. Mutants showing distorted blue coloring on plates containing X-gal were selected and analyzed in more detail as described in results.

Construction of a codY Deletion Strain.

A 1400 bp EcoRI/HinDIII chromosomal fragment of L. lactis MG1363, containing codY, was subcloned in pUC19. The resulting plasmid was digested with SnaBI and subsequently selfligated. In this way, 423 bp were deleted from codY The oligonucleotides cod280A (5′ GGGAATTCGGATTGTCTATCTGCCTCG 3′) and cod280B (5′ GGGGGATCCAGATCTGACCATGATTACGCCAAGCTT 3) were used to amplify the ΔcodY-containing fragment. PCR product was digested with EcoRI/BamHI (restriction sites are underlined in the oligonucleotide sequence) and ligated into corresponding sites in pORI280. The resulting plasmid, pORIΔcodY, was introduced together with pVE6007 into L. lactis MG1363. As this strain does not contain the repA gene, selection for growth in the presence of erythromycin and increased temperature (37° C.) forces pORIΔcodY to integrate into the chromosome by homologous recombination. A number of integrants were subsequently grown for about 30 generations under nonselective conditions allowing a second recombination event to occur, which results in either the deletion or the wild-type gene codY. The ΔCodY mutation was confirmed by PCR.

β-Galactosidase Activity Assay

In vivo β-galactosidase (β-gal) assays were carried out in a Tecan microplate reader. Overnight cultures of L. lactis grown in GM17 were washed twice in 0.9% NaCl before inoculation to 2.5% in 200 μl of the appropriate medium containing erythromycin (5 μg/ml) for maintenance of pORI13 in L. lactis LL108/LL302 and erythromycin and chloramphenicol (2.5 μg/ml each) (Leenhouts et al., 1996). The media also contained the β-gal substrate 2% β-triiluoromethylumbelliferyl β-D-galactopyranoside (Molecular probe T-657). Multilabelling experimental data (absorbance and fluorescence measurements) were processed using the Magellan software program. β-Gal production due to the transcription driven from the oppD upstream region was calculated as a function of light emission. β-Gal assays were performed throughout the growth of L. lactis grown in media in which growth rates differ significantly as a function of the nitrogen source i.e. CDM 0.2% casitone and CDM 2% casitone and measured of the culture. β-Galactosidase activities were determined in permeabilized cell suspensions as described previously (Israelsen et al, 1995). β-Galactosidase enzyme activities, calculated as an average of three independent experiments, were expressed in arbitrary units (Miller, 1972).

Overproduction and Purification of His₆-CodY.

The chromosomaly located codY of L. lactis MG1363 was amplified by PCR with the oligonucleotides HC-5 (5′ CTAGACCACCATGGGG CATCACCATCACCATCACGTGGCTACATTACTTGAAAAAACACG 3′), introducing the underlined NcoI restriction enzyme site upstream of the hexa-histidine tag (italic) and HC-6 (5′ CTAGTCTAGATTAGAAATTACGTCCAGCAAGTTTATC 3′), introducing the underlined XbaI restriction enzyme site downstream of the stop codon (italic) of codY. The purified 833-bp PCR product was digested with NcoI and XbaI and ligated into the corresponding sites of pNZ8048, downstream of the nisin-inducible P_{nisA}. The resulting plasmid, pNH6CodY, was introduced in L. lactis NZ9000 to enable nisin induction of his6-codY, as described (de Ruyter, Kuipers, and de Vos, 1996; Kuipers et al., 1998). His6-CodY was isolated by affinity chromatography in an FPLC procedure (Amersham Pharmacia Biotech) using Ni-NTA agarose (Qiagen GmbH, Hilden, Germany).

Electrophoretic Mobility Shift Assays (EMSA's)

Gel retardation experiments were carried out essentially as described by Ebbole and Zalkin (Ebbole et al., 1989). Purified PCR products (2 μg) were end-labelled with polynucleotide kinase (Amersham Pharmacia Biotech) for 1 h at 37° C. using 30 μCi [γ-³²P]-ATP (Amersham Pharmacia Biotech) in a volume of 20 μl. Reactions were stopped by incubating the mixtures for 10 min at 70° C. Binding studies were carried out in 20 μl reaction volumes containing 20 mM Tris-HCl (pH 8.0), 8.7% (v/v) glycerol, 1 mM EDTA (pH 8.0), 5 mM MgCl₂, 100 mM KCL, 0.5 mM DTT, labelled DNA fragment (3000 cpm), and purified His6-CodY protein (50-400 ng). BSA (1 μg) and poly(dI-dC) (Amersham Pharmacia Biotech) were added to the reaction mixtures in order to reduce non-specific interactions. After incubation for 15 min at 30° C., samples were loaded onto a 4% polyacrylamide gel. Electrophoresis was performed in the Protean II Minigel System (Bio Rad Laboratories B. V., Veenendaal, The Netherlands) using a gradient (0.5× to 2×) of TAE buffer (Sambrook, Fritsch, and Maniatis, 1989) at 150 V for 1.5 h. Gels were dried and used for autoradiography at −80° C. using Kodak XAR-5 films and intensifying screens.

Preparation of Cells for Transcriptome Analysis

Cells were grown at 30° C. in GM17 supplemented with 0.5% glucose. Cells were grown till mid-exponential phase (OD600˜1.0). Approximately 5×10⁹ cells (50 ml culture) were harvested by centrifugation for 5 min at 10.000 rpm and 4° C. Cells were resuspended in 2 ml ice-cold growth media and divided over 4 screw-cap tubes with rubber seal. After the addition of 500 μl Phenol/Chloroform, 30 μl 10% SDS, 30 μl 3 M NaAc (pH5.2) and 500 mg glass-beads (diameter 75-150 μm), cells were frozen in liquid nitrogen and stored at −80° C. or immediately used for RNA isolation.

Transcriptome Analysis

The DNA micro array experiments were essentially performed as described earlier (Kuipers et al., 2002), with the following modifications.

RNA Isolation

For each RNA isolation, one aliquot of the stored cell samples was used. Cells were disrupted by mechanical force using the Savant FastPrep FP120 system (Omnilabo) for 40 seconds at setting 5.0. Subsequently, RNA was extracted using the Roche “High Pure RNA Isolation Kit” according to the provided protocol. RNA yield and quality were determined spectrophotometrically and by performing a RNA 6000 Nano Labchip assay (Caliper Inc.) on the Agilent 2100 Bioanalyzer (Agilent Technologies, Amstelveen, the Netherlands) according to the manufacturers description respectively.

cDNA Labeling

Single-strand reverse transcription (amplification) and indirect labeling of 25 μg of isolated total RNA with either Cy3- or Cy5-dye were done with the Amersham CyScribe Post Labelling Kit according to the manufacturers protocol and, subsequently, used for hybridization.

Hybridisation and Scanning

Sylilated slides (Cel Associates) on which 2145 amplicons of L. lactis strain IL1403 were spotted in duplicate were used in the hybridization procedure. In addition, the slides contained 96 amplicons from L. lactis strain MG1363. Slides were pre-hybridized in Ambion SlideHyb buffer for 15 min at 40° C. in a Genomic Solutions Hybstation. After removal of the pre-hybridization buffer, 10-25 μl of the Cy3/Cy5-labeled cDNA mix in 150 μl Ambion SlideHyb buffer I was added and incubated for 1 h at 42° C. and finally for 16 h at 40° C. Afterwards, the hybridized slides were washed for 1 min in 2×SSC, 0.5% SDS and 5 min in 1×SSC, 0.25% SDS. The slides were scanned using a confocal laser scanner GeneTAC LS IV).

Signal Analysis

After scanning of the slides with the GeneTAC LS IV, individual spot intensities were determined. The raw data, along with the scanning image were stored in the Molecular Genetics Information System (MolGenIS). A grid definition was made to enable the spot analysis software Array Pro (Phoretix) to produce tables containing gene names and signal intensities. Using the program Excel Microsoft corporation), signal intensities were corrected for background and the ratios in signal intensity between the different samples were determined.

DNaseI Footprinting Analysis

DNaseI footprinting was performed essentially according to the description supplied with the Sure Track Footprinting Kit (Amersham Pharmacia Biotech). The DNA fragments were prepared by PCR, using Expand DNA polymerase (Amersham Pharmacia Biotech) and the oligonucleotides opp1 and opp3, one of which was first end-labeled with T4 polynucleotide kinase (Amersham Pharmacia Biotech) and [γ-³²P]ATP as described by the manufacturer. Binding reactions were identical to those used in EMSAs, in a total volume of 40 μl and in the presence of approximately 150,000 cpm of DNA probe. DNAseI footprinting experiments were then performed as described previously (Hamoen et al, 1998)

In Vitro Protein Cross-linking 5

Cross-linking reactions were performed in 1× cross-linking buffer (100 mM KCl, 15 mM Tris-HCl pH 7.5) in a total volume of 40 μl, containing 300 ng of purified H6-CodY, and, where indicated, GMP (1 mM) or GTP (1 mM). Formaldehyde was used as the cross-linking agent at a final concentration of 1% (v/v), with a 10 min incubation step at room temperature. Cross-linking reactions were stopped by the addition of 20 μl of 2×SDS gel loading buffer [4% SDS, 10% 2-mercaptoethanol, 20% glycerol, 100 mM Tris-HCl (pH 6.0)]. Samples were boiled for 5 min before separation by SDS polyacrylamide (10%) gel electrophoresis (Laemmli, 1970). SDS polyacrylamide gels were stained with Coomassie Brilliant Blue (Bio-Rad).

### EXPERIMENTAL PART

### Results

Determination of the Transcription Start Site of oppD.

It has been demonstrated that the genes of the oligopeptide permease system, encoded in the oppDFBCA-pepO1 locus of L. lactis MG1363, are transcribed polycistronically. Upstream of both oppD and oppA regions are present that could serve as promoter elements. To determine the location of the promoter upstream of oppD, the transcription start site (TSS) was determined. The opp transcript was analyzed by primer extension, using RNA that was isolated from exponentially growing MG1363 cells. The mRNA 5′ end corresponds to an adenine residue located 35 bases upstream of the translation start codon AUG of oppD. The −35 sequence (TTGCAA) is separated by a consensus 17 bases from the −10 region (TATACT) and a proper lactococcal ribosome binding site; GAGG is also present. The sequence upstream of oppD contains two regions of dyad symmetry centered around position −135 (−14.0 kcal/mol) and −62 (−5.6 kcal/mol) relative to the oppD transcriptional start site, respectively.

CodY Specifically Binds to the oppD Upstream Region.

In order to examine whether L. lactis CodY directly interacts with upstream DNA sequences of its main target known so far, oppD, in vitro DNA binding studies were performed. For this purpose, histidine-tagged CodY was overexpressed using the nisin inducible gene expression system (Kuipers et al., 1998) and subsequently purified to apparent homogeneity. A radioactively labeled PCR fragment spanning the oppD upstream region (FIG. 1) was used as a probe. The electrophoretic mobility shift assays (EMSA's) clearly showed that purified H6-CodY is capable of binding directly to a region encompassing the oppD promoter (FIG. 2). Multiple retarded fragments were observed, indicating that, probably, multimerization of H6-CodY on the oppD promoter occurs.

Upon gradually increasing the amount of H6-CodY from 10 to 100 ng, four distinct bands can be observed, indicating a tetrameric state of the protein. In most of our DNA binding experiments, a band corresponding to single stranded probe (s.s. DNA) was observed irrespective of the presence of H6-CodY. The occurrence of this denatured DNA probably results from the high AT content of the opp promoter region. No H6-CodY binding occurred when DNA fragments, with similar AT contents, were used that were obtained from internal gene segments (e.g. from comg of B. subtilis), indicating that H6-CodY binding to PoppD is specific (data not shown).

CodY Regulates the Expression of oppD by Binding to a Specific Upstream Sequence.

The upstream oppD region contains a small sequence that is inversely repeated with a spacing of 9 base pairs in between (FIG. 1). This region of dyad symmetry is located just upstream of the −35 promoter sequence. To determine whether this region is important for the interaction with CodY, a stepwise deletion analysis of the oppD upstream region was performed. Radioactively labeled deletion fragments of the region, obtained by PCR, were incubated with H6-CodY. As shown in FIG. 3, the fragments with an extended deletion of part of the oppD upstream region showed altered binding of CodY. Two truncated fragments, shortened from the 5′-end of the oppD upstream region contained in opp162, were obtained by PCR and examined for H6-CodY binding (FIG. 3A and 3B). Probe 2, spanning the region from −111 to +75, was still bound by H6-CodY, suggesting that nucleotides that are critical for CodY binding must reside downstream of the 5′-end of this probe. When a 160-bp DNA fragment was used, of which the 5′-end coincides with the center of the inverted repeated closest to the RBS of oppD (FIG. 1B), no binding of H6-CodY was observed, irrespective of the amount of protein added (probe 1 in FIG. 3B).

As the fragments used in the EMSA's were also cloned upstream of the promoterless lacZ gene in plasmid pORI13, the in vivo regulation of a downstream reporter gene (lacZ) could be determined by performing β-galactosidase (β-gal) assays in a chemically defined medium (Poolman and Konings, 1988) containing 2% of casitone as a nitrogen source. It was shown that deletion of the sequence abolished medium dependent repression of lacZ expression. the fragments were fused upstream of the promoterless lacZ gene in plasmid pORI13 (Sanders et al, 1998) and introduced in L. lactis LL108. β-Galactosidase activities in exponential phase cells growing in CDM supplemented with 2% casitone, where strong CodY-mediated repression is ensured, correlated with the observed binding pattern (FIG. 3C), indicating that at least part of an operator site for CodY must rely in a region between positions −111 and −68 relative to the oppD TSS.

The introduction of base substitutions in the upstream half-site of the inverted repeat gave rise to both weaker binding of H6-CodY and resulted in derepression of the promoter as shown by gel retardation analysis and β-gal assays, respectively (FIG. 4).

H6-CodY Protects an Extended Region of the oppD Promoter.

Although the EMSA experiments using truncated PoppD DNA fragments pointed to a specific region that is important for CodY binding, the actual area facilitating binding was rather large (position −68 to −111). Moreover, the assays did not exclude the possibility that more downstream sequences could contribute to CodY binding. Therefore, complex formation of H6-CodY with the oppD promoter region was investigated by DNaseI footprinting experiments, using the labeled promoter fragment (opp162) and binding conditions as used for the EMSAs (FIG. 10). H6-CodY binding results in the protection of bases extending from the −80 to −20 and −80 to −10 positions relative to the transcription start site of the upper and lower strand, respectively. In addition to these protected regions, both DNA strands contained hypersensitive sites when they were incubated with DNaseI in the presence of H6-CodY. These results show that H6-CodY binds to a region encompassing the −35 to −10 sequences of the promoter of oppD.

A Region in the oppD Promoter Containing an Inverted Repeat is Important for CodY-mediated Regulation.

A closer inspection of the area in the oppD promoter to which CodY binds revealed the presence of a short sequence (ATGTTCA) that is inversely repeated (IR) with a spacing of 9 bp between the partners (FIG. 1). This region of dyad symmetry is located 18 bp upstream of the −35 sequence and is entirely present in probes 2 and 3 to which H6-CodY bound (FIG. 3). The fact that H6-CodY was unable to form a protein-DNA complex when a probe was used that lacks the upstream arm of this IR (probe 1 in FIG. 3) implies that this area might serve as an operator site for CodY on PoppD. To study this region in more detail, site directed mutations were introduced by PCR using combinations of oligonucleotide opp1 with oligonucleotides opp14 (WT), opp15(a), opp(15b) or opp2, respectively (FIG. 4A). The PCR products were ligated upstream of the promoterless lacZ reporter gene in pORI13 and introduced into L. lactis LL108. Introducing base substitutions in the upstream arm of the repeat (opp15(a) and opp15(b)) resulted in both weaker binding of H6-CodY (FIG. 4C) and in derepression of PoppD-driven lacZ expression (FIG. 4D). When the unchanged half-site was replaced by an unrelated sequence (an XbaI endonuclease site, present in opp2), repression was reduced approximately 30-fold (in cells growing exponentially in rich medium) and H6-CodY binding was completely abolished (as shown in FIG. 3). Converting both the C and G residues in this region to adenines resulted in a reduction of repression of about 7-fold (opp15(a)), whereas changing 3 out of 6 bases (opp15(b)) led to a derepression of expression of more than 20 times. These results were in accordance with those obtained from gel retardation analyses (FIG. 4C), where the order of affinity of H6-CodY for the probes proved to be: WTprobe>opp115(a)>opp15(b). In case of the latter probe, hardly any protein-DNA complexes were present and all intermediate complexes that were observed with the WT and opp15(a) fragments were absent.

Random Mutation analysis of the oppD Upstream Region.

In order to identify bindings sites of CodY in the oppD upstream region, random mutation was carried out on the smallest oppD promoter fragment that is still bound by CodY (see FIG. 3). By choosing PCR conditions that allow mismatches to occur during DNA amplification, DNA fragments spanning the oppD promoter region were obtained containing randomly introduced base pair substitutions. The fragments were restricted using the appropriate restriction enzymes, cloned upstream of the promoterless lacZ reporter gene in pORI13 and introduced in L. lactis LL108. Transformants that show a derepressed phenotype appear as white or light blue colonies on agarplates containing excess nitrogen sources (2.5% of casitone) and X-gal. Mutants that showed a distorted blue coloring on plates were tested for the ability to complex with CodY in a gel retardation experiment. The strength of binding was determined by comparing the amount of shifted mutated DNA fragment as compared to that of the corresponding wild type fragment (FIG. 5). Weaker binding to all of the mutated promoter fragments was observed. Strikingly, all the mutants obtained carried substitutions in one or both of the half-sites of the inverted repeat present in the oppD upstream region, indicating the importance of this sequence in CodY binding. Sequence analysesof the PoppD variants obtained in this study revealed that all of them carried one or more base pair substitution(s), of which at least one is located in the region from −82 to −56 relative to the TSS of oppD. This, again, is an indication of the importance of this region for CodY binding.

Identification of Additional CodY Targets.

In order to identify additional genes that are regulated by CodY, the transcriptional profile of wild-type (WT) L. lactis MG1363 was compared with that of a L. lactis MG1363 strain containing a 423 bps internal deletion in the codY gene using DNA micro arrays containing genes of L. latis IL1403. These studies revealed several differentially expressed genes in the delta codY strain. The genes of which the expression is increased most significantly in the delta codY strain are listed in Table 2. The genes of which the expression is decreased most significantly in the absence of a CodY-like protein are listed in Table 3. As this increased expression (Table 2) could be a direct effect of the absence of CodY (derepression), the upstream regions were examined- for the presence of conserved nucleotide sequences. It was found that many of these genes contain an upstream sequence that is homologous to the upstream half-site of the palindromic sequence found to be important in oppD regulation (FIG. 6A). Interestingly, the upstream region of the gene that shows the highest fold difference in expression when comparing the codY strain to WT L. lactis MG1363, optS, contains two copies of this sequence. The sequence identified is not in all instances part of an inverted repeat. At a later stage, the experiment was repeated. Results are listed in table 10.

The conserved sequence could not be discerned in the upstream regions of all the differentially expressed genes. This could mean that the altered expression in the delta codY strain is an indirect effect of the mutation or that other sequences or structural determinants also play a role in the recognition/regulation by CodY. As can be seen in FIG. 7 several other conserved motifs could be identified in the upstream regions of several differentially expressed genes. These sequences could play a role in CodY-regulated expression (e.g. in the case of prtP and prtM).

Using a weight matrix constructed from the sequences that comprise the putative CodY box, a search was performed in a data set containing the upstream regions of all IL1403 genes (Table 4). The presence of such an element could indicate that the downstream gene is under direct transcriptional control of CodY.

An Overrepresented Motif is Present in the Upstream Regions of a Number of the CodY-repressed Genes

In order to assess whether the lactococcal members of the CodY regulon share a sequence motif in their regulatory regions, an in silico sequence analysis was performed using the algorithm of the MEME software tool. Firstly, a dataset was created containing the upstream regions of the genes that were derepressed to the highest extent in the codY strain in our DNA microarray experiments, as we assumed that these genes are most likely to be under direct control of CodY and thus contain a CodY binding site. This dataset was supplemented with the upstream regions of genes of which it is known that they are under direct control of CodY (i.e. oppD, pepC, pepN) as is the case for the intergenic region of the divergently transcribed L. lactis subsp. cremoris SK11 plasmid-located prtP and prtM genes for which it has been shown that purified H6-CodY of the closely related lactococcal strain MG1363 is able to interact with DNA fragments spanning this region in in vitro DNA binding studies. Since operator sites of regulatory proteins in bacteria are usually located within close proximity of their target promoters, fragments of 200 bps were chosen such that they encompassed the known or predicted promoter sequences of their cognate genes. In cases were a complete operon was found to be up-regulated in the codY deletion strain, the upstream region of the first gene of the transcriptional unit was selected (i.e ilv, his, opp and dpp). Of the latter operon, the region preceding dppA was also included in the dataset, since a putative promoter sequence is present in this area that, thus, might contain a CodY binding site as well.

Subsequently, the dataset containing 14 sequences was examined for the occurrence of common elements using the MEME algorithm. As there is no prior knowledge about a possible CodY binding site, MEME was not restricted with respect to the motif width and number of repetitions and allowed to search on either of the given or the reverse DNA strand. Moreover, these settings did prevent that the common upstream elements (−35 and −10 sequences and the ribosome binding site) would conceal the presence of a CodY binding site. As can be seen from Table 4A and FIG. 6B, application of the pattern recognition program approach revealed the presence of a distinct over-represented motif in a number of the DNA sequences of the dataset. Derivates of this 15 bps AATTTTCWGAAAATT inversely repeated (IR) cis-element are present in the upstream regions of 11 out of the 14 co-regulated genes that constitute the input data for the program. In the upstream regions of udp, hisC, and pepN this over-represented box in CodY-co-regulated genes (C-box) seems to be absent.

Interestingly, the upstream region of the operons that show the highest fold difference in expression when comparing the codY strain to WT L. lactis MG1363, dpp and ilv, contain two copies of this C-box preceding both dppP, dpp and ilvD, encoding the peptide binding proteins of the uptake system and the three branched-chain amino acid biosynthesis genes, respectively, whereas in case of ctrA and in front of gltA, even 3 copies of the motif can be found.

Inter-species Analysis of the over Represented Motif

A multi-species string search was performed on several Gram-positive bacteria in order to assess whether the putative CodY box found upstream of the genes belonging to the CodY regulon is also present in other bacteria containing a CodY protein. Such a comparison could reveal subtle differences in the consensus sequence of this motif. As depicted in FIG. 7, the comparison indeed showed that derivatives of the motif are present in the upstream sequences of CodY regulated genes of B. subtilis (e.g. dppA and hutP). FIG. 6A shows a graphical representation of a species-specific “weight matrix” that was built using the aligned sequences of L. lactis or B. subtilis, respectively. These matrices indicate the importance of specific bases at each position in the motif and show that the motif detected in L. lactis seems to be highly similar to the one of B. subtilis. Both encompass the same “core” sequence, but the consensus of the L. lactis motif seems to be somewhat extended on both sides.

Recently, Molle et al. (2003) reported the genome wide expression analysis of a B. subtilis codY mutant strain. Using the upstream nucleotide sequences of the targets found in that study, we searched for the presence of the putative CodY box in these sequences. Although the similarity scores with the consensus where not very high, derivates of the motif could be identified in some instances in this set of sequences (Table 5). Analysis of the full genome sequence of B. subtilis resulted in the identification of additional promoters containing the putative CodY box (Table 6).

A similar search was performed using the genomes of several other Gram-positive bacteria (Tables 7 and 8). Currently, we are mining the genome of L. lactis strain MG1363 of which the genome sequence is nearly completed.

Regulation of prtP and prtM is Derepressed in the CodY-deficient Strain.

As prtP and prtM respond in a similar way to changes in the nitrogen content of the growth medium as CodY-regulated genes (Miladinov, Kuipers, and Topisirovic, 2001; Guedon et al., 2001 a), the role of CodY in prtP and prtM expression was studied. A fragment containing the prtP/prtM intergenic region of L. lactis BGMN1-5 was cloned in between the promoterless Escherichia coli β-galactosidase (lacZ) and the Cyamopsis tetragonoloba α-galactosidase (α-gal) genes of pGKH10. Thus, translational fusions of prtP and prtM with the two reporter genes were created, the AUG codons of prtP or prtM serving as a start codon for lacZ or α-gal. In the resulting plasmid pGKB11, lacZ is under the control of the prtP promoter of BGMN1-5. In this plasmid the prtM promoter directs the transcription of the α-gal gene. In the corresponding plasmid pGKB12, the fragment is present in the opposite orientation. Plasmids pGKB11 and pGKB12 were introduced in the CodY-deficient L. lactis MG1363 strain (L. lactis MG1363codYΔ1) and lacZ expression was analysed in CDM media containing different amounts of casitone, and in peptide-rich M17 medium. In the WT strain, the β-gal activities of the prtP::lacZ and prtM::lacZ fusions were 6- and 8-fold lower in CDM with 2% than in CDM with 0.2% casitone, respectively. In the codY mutant, β-gal activity in CDM with 2% casitone was less than twofold lower than that in CDM with 0.2% casitone. β-gal activities in M17 were similar to those in CDM with 2% casitone for both MG1363 and MG1363codYΔ1. These results show that repression of these two gene fusions by medium peptides was almost fully abolished in the codY mutant.

CodY Binds to the prtP/prtM Intergenic Region.

The prtP/prtM intergenic regions of the L. lactis strains BGMN1-5, E8, SK11 and Wg2 each contain an inverted repeat (FIG. 8), that is highly conserved on the sequential level (Marugg et al., 1996). To determine whether CodY is also able to recognize and bind to the prtP/prtM intergenic region, gel mobility shift assays were performed using purified H6-CodY. A 330-bp γ-³²P-labelled PCR fragment containing the prtP/prtM intergenic region derived from L. lactis BGMN1-5, E8, SK11 and Wg2, respectively, were used as probes. Addition of His6-CodY resulted in a markedly lower electrophoretic mobility of all four double stranded PCR products on a polyacrylamide gel as compared to the situation in which CodY was not added (FIG. 9). Moreover, multiple shifted bands are present suggesting that CodY migh act as a multimer. These results indicate that CodY directly binds to the prtP/prtM intergenic regions of all four lactococcal strains tested. Since the inverted repeats present in the prtP/prtM intergenic region overlaps the −10 sequence of both the prtP and prtM promoter, they could function as a binding site for CodY, thereby blocking transcription of both genes simultaneously.

H6-CodY Forms Multimers in the Absence of GTP.

In order to identify whether the active form of H6-CodY, a protein of 29 kDa, consists of mono- or multimers, cross-linking experiments were carried out on purified H6-CodY. Initial experiments, using glutaraldehyde as a cross-linking reagent proved to be unsuccessful (data not shown). The results using formaldehyde as a cross-linking agent are shown in FIG. 11 and show that oligomeric forms of H6-CodY are present. Covalently linked CodY complexes of sizes close to those expected for dimeric (58 kDa) and, less pronounced, tetrameric H6-CodY molecules (116 kDa) were obtained. Formation of cross-linked H6-CodY complexes was not significantly stimulated by the presence of GTP, a molecule that serves as a cofactor of B. subtilis CodY. These results indicate that the native form of CodY is a di- or tetramer.

BCAAs Affect H6-CodY Binding Properties. 22

Evidence has been presented that CodY senses the nitrogen supply of the cell as a function of the BCAA pool. Although BCAAs might act as direct effectors of CodY activity, the exact nature of this signal remains to be established. Therefore, in vitro DNA binding of H6-CodY was examined in the presence or absence of the three BCAAs Val, Leu and Ile (FIG. 12). The addition of any of the three BCAAs resulted in severely altered binding of H6-CodY to the PoppD probe 3 as compared to the situation in which no or another, aliphatic, amino acid (i.e. alanine) was present. All of the probe DNA was retarded in the presence of Ile at concentrations of both 0.6 and 2.5 mM, showing that this amino acid directly stimulates the binding of H6-CodY to its target. Strikingly, in the presence of Val and Leu, H6-CodY behaved rather differently. The addition of either of these BCAAs to a final concentration of 0.6 mM resulted in a reduction of the amount of shifted, H6-CodY-bound, probe DNA. Increasing the concentration of these two amino acids to 2.5 mM led to the formation of a protein-DNA complex of high molecular weight, whereas the capability of H6-CodY to form complexes of lower molecular weight seems to be lost.

Similarly, we also tested whether GTP could stimulate the binding of H6-CodY to lactococcal PoppD, since in B. subtilis GTP serves as an effector molecule that enhances binding of bacillus CodY to a number of its targets. However, no effect is seen on lactococcal CodY at a concentration of 2.5 mM GTP (right lane in FIG. 12) as well as at other concentrations (data not shown).

CodY Directly Complexes with the asnB and ctrA Upstream Regions

To distinguish whether repression by CodY occurs directly or indirectly, an electric mobility shift assay (EMSA) was performed in which purified H6-CodY was used. FIG. 13 shows that H6-CodY, like in the case of the well-studied oppD promoter, interacted with a radioactively labeled DNA probe spanning the asnB promoter in vitro.

This result, together with those of the in vivo transcription assays indicate that expression of asnB is probably under the direct control of CodY. Using EMSA's, direct interaction of H6-CodY was also shown for a probe encompassing the upstream region of ctrA. As is apparent from Table 2 and Table 10, the transcript level of ctrA, a gene encoding a putative transporter with homology to several cationic amino acid permeases is highly elevated in L. lactis MG1363Dcody).

### DESCRIPTION OF FIGURES

FIG. 1 Overview of the opp-pepO1 operon.

Panel A shows a schematic overview of the opp-pepO1 operon. Panel B shows a detailed view of the upstream sequence encompassing the oppD promoter. The −35, −10 and ribosome binding site (RBS) sequences are underlined. The arrows show the positions of the two pairs of inverted repeats. The inverted repeat that is discussed in the text in more detail is indicated in bold.

FIG. 2 His6-CodY binds to the oppD promoter.

Gel retardation assay using His6-CodY and the oppD promoter fragment. A DNA fragment encompassing the oppD promoter was amplified by PCR, radioactively labelled and incubated with no (lane 2) or increasing amounts of purified H6-CodY (lanes 3 to 9). Lane 1 contains the same probe, but was boiled in a 95% formamide solution in order to obtain single stranded DNA fragments.

FIG. 3 Determination of the minimal region involved in CodY binding to the oppD upstream promoter region.

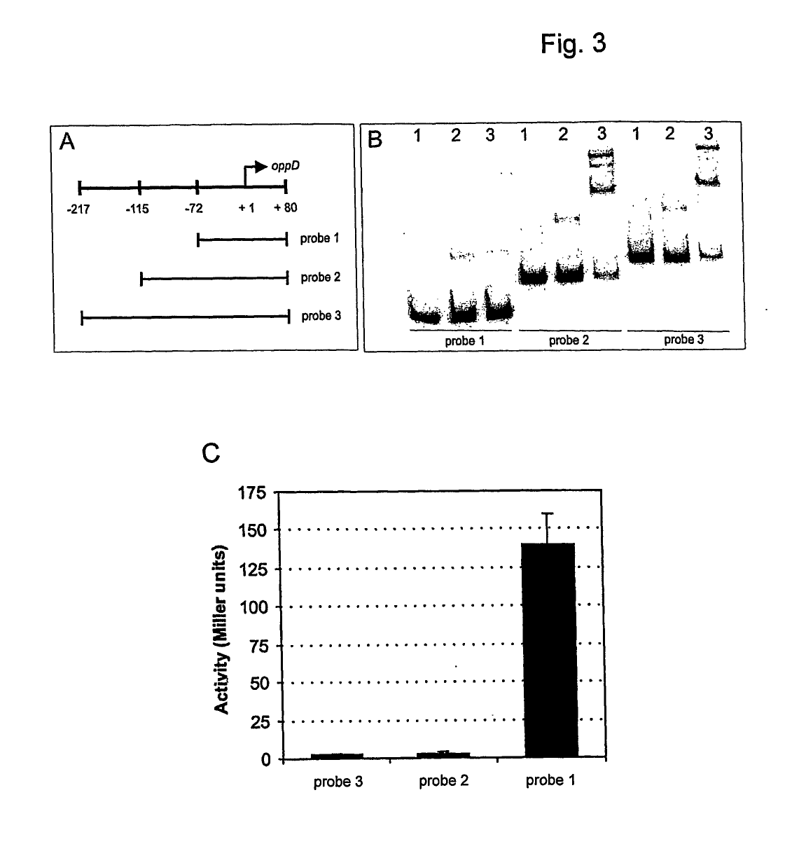

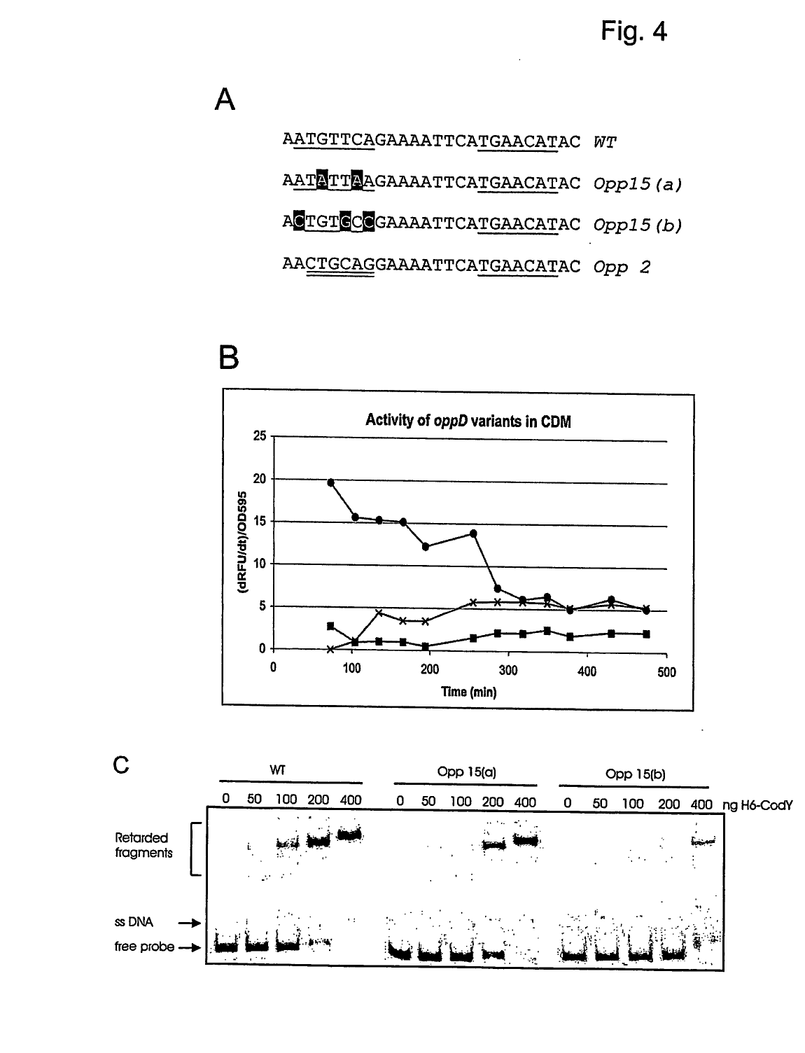

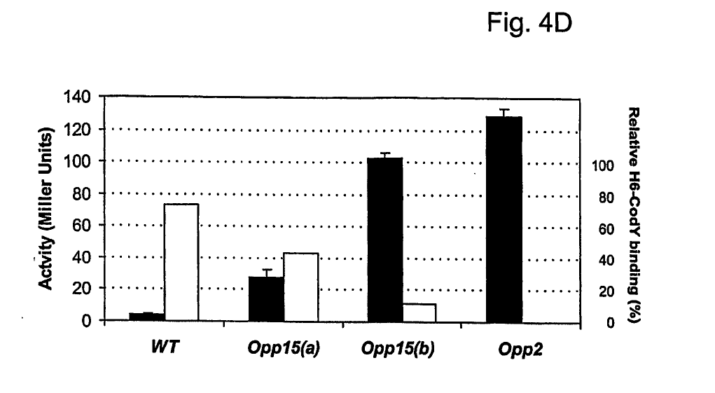

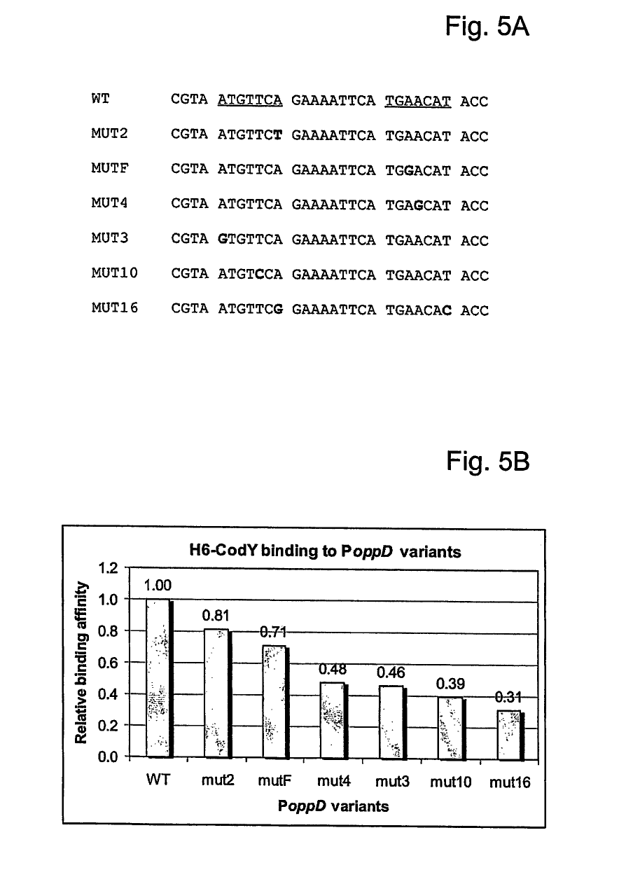

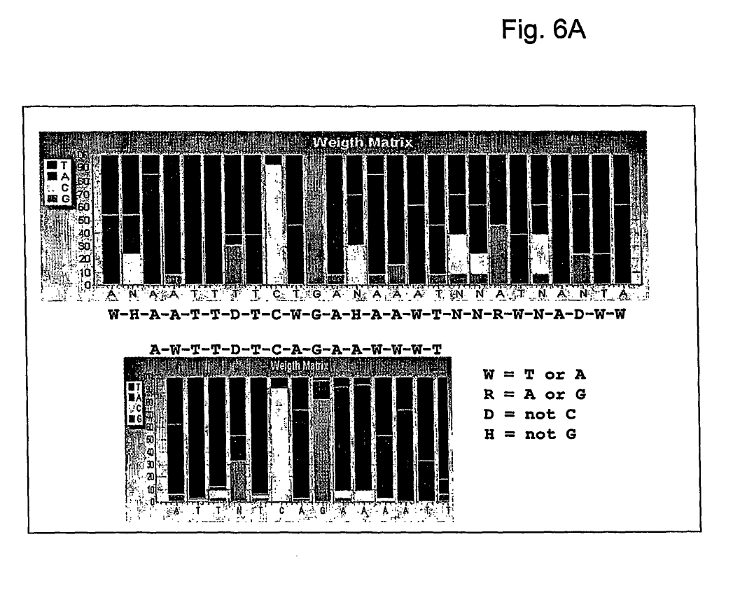

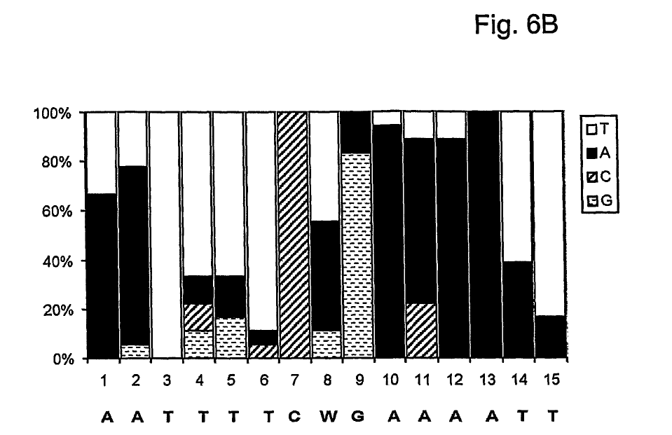

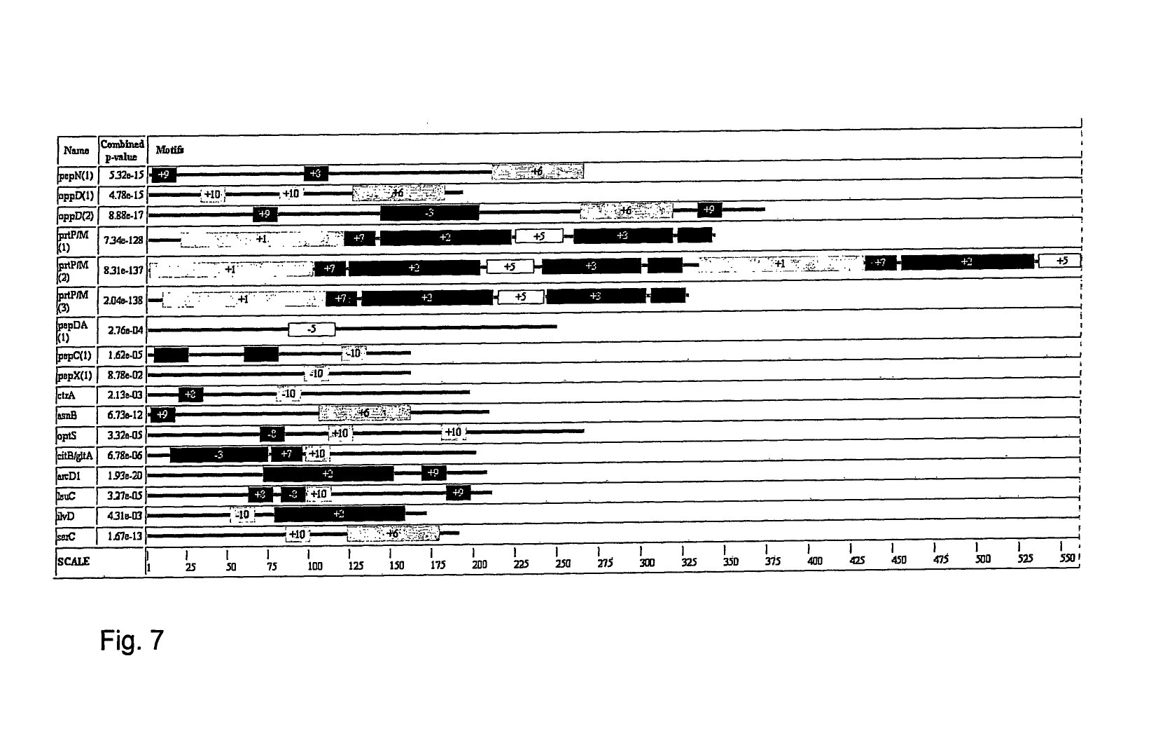

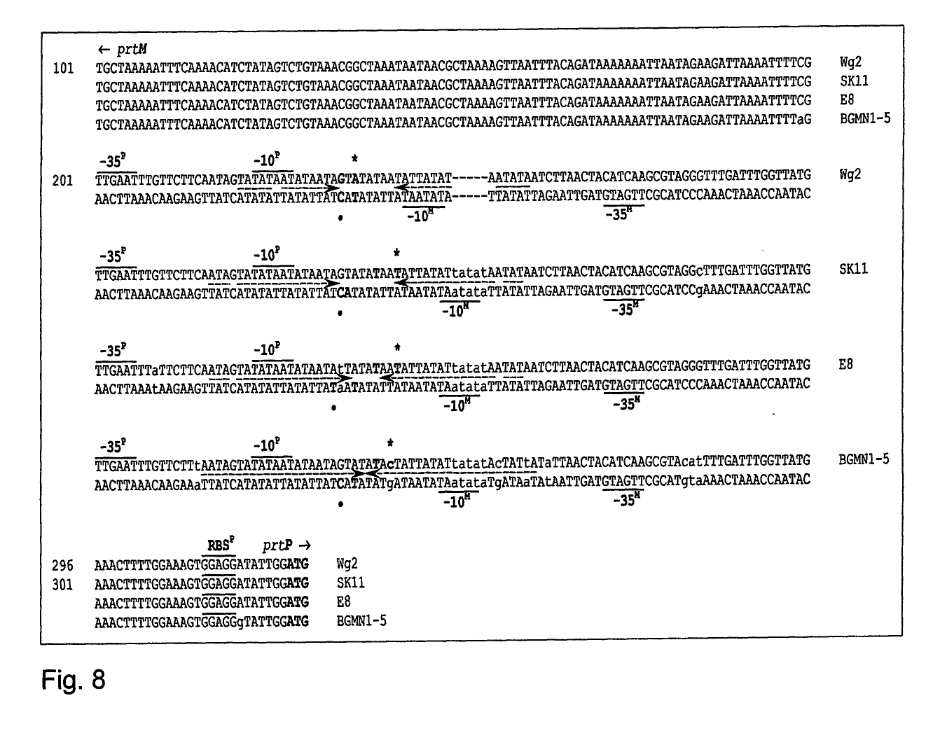

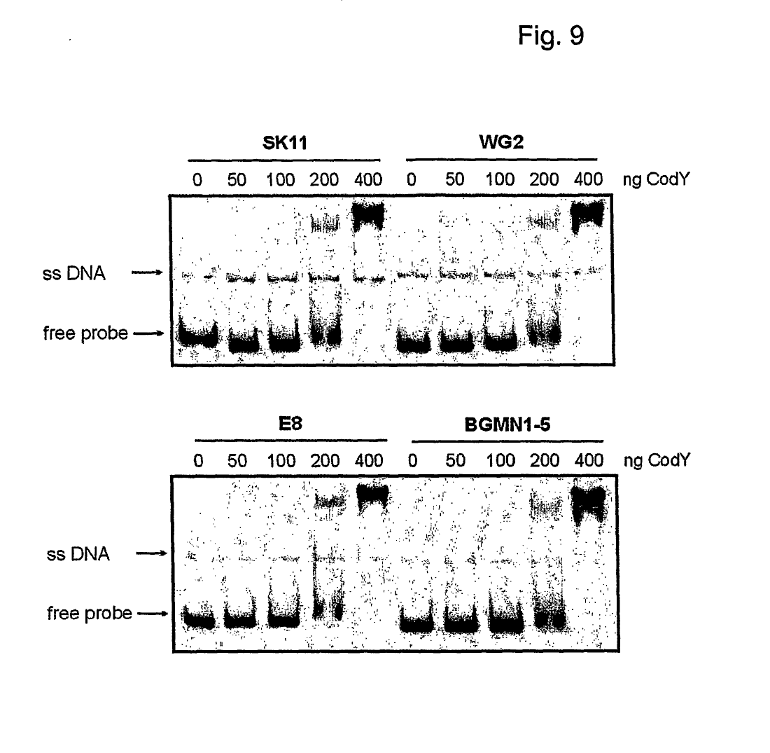

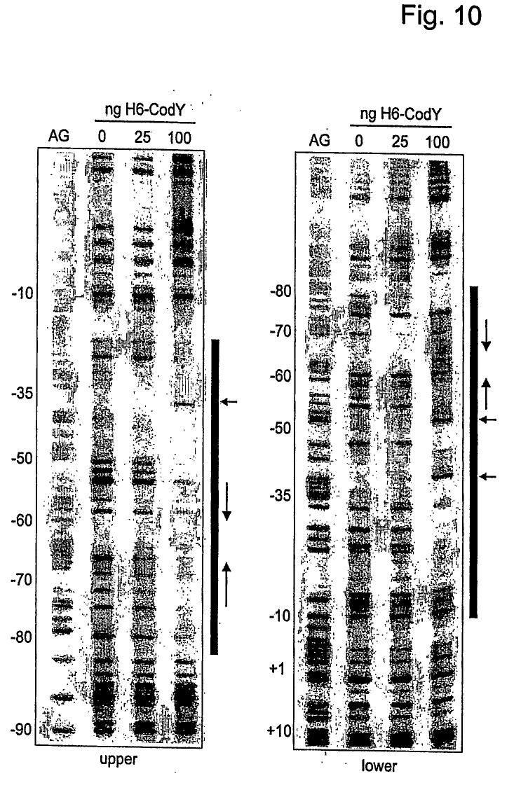

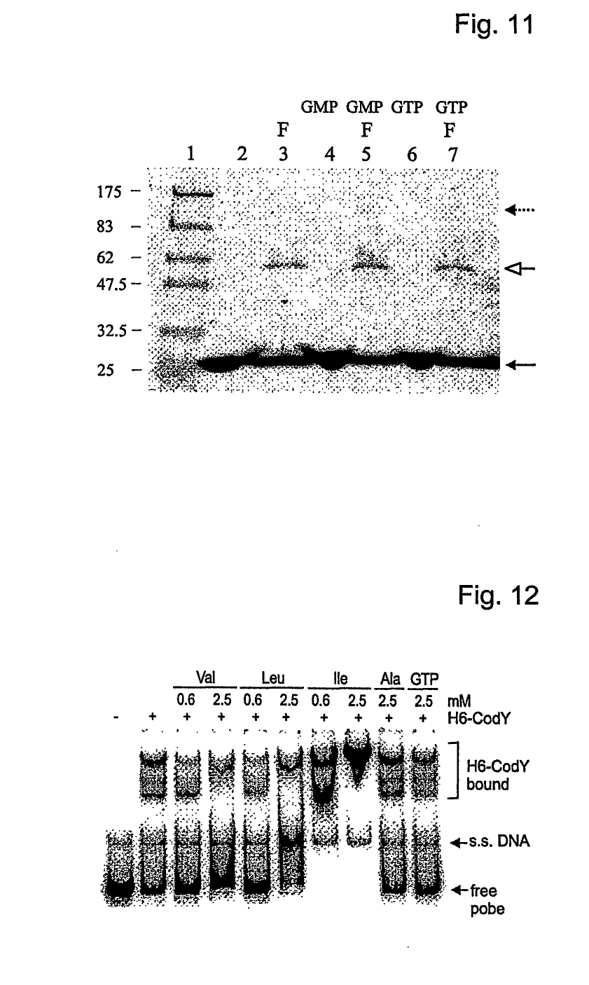

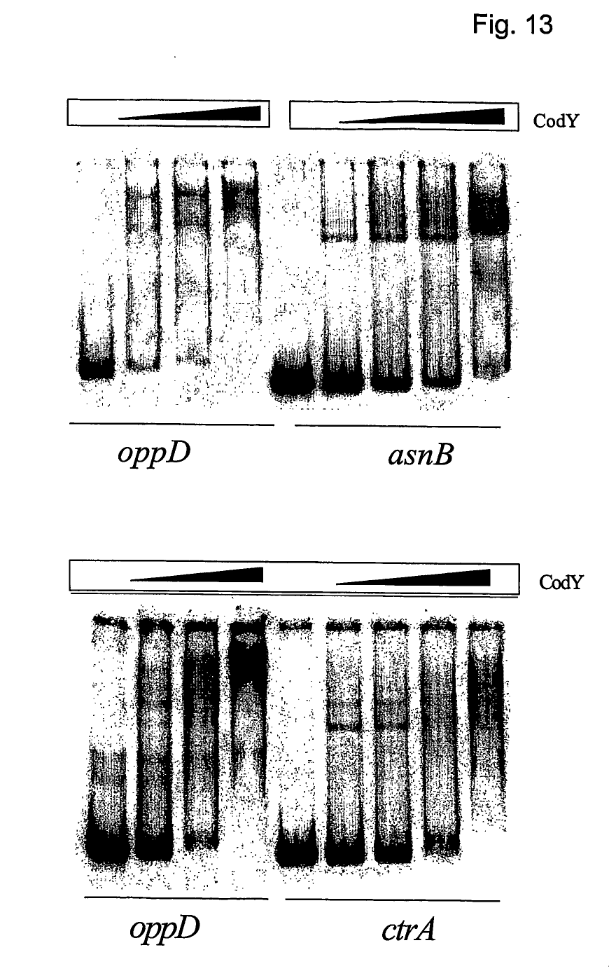

Different parts of the oppD promoter region were amplified by PCR (panel A), radioactively labelled and incubated with no (lanes 1), 20 ng (lanes 2) or 200 ng (lanes 3) of His6-CodY protein, respectively (panel B). FIG. 3C: In vivo activity of PoppD variants. L. lactis LL108 strains carrying lacZ reporter plasmids fused the opp fragments depicted in panel A were grown in CDM containing 2% casitone. Cells were harvested in the exponential phase of growth and β-galactosidase activity was measured (solid bars). The experiments were carried out in triplicate. Error bars indicate standard deviations.

FIG. 4 Effects of site-directed mutations in the area of inverted repeat in the oppD upstream region. Three constructs (panel A) were compared for both β-gal activity (panel B) and H6-CodY binding (panel C). WT fragments contain no substitutions, whereas fragments Opp15 (a) and Opp15 (b) contain 2 and 3 mutations, respectively (indicated in bold in panel A). In opp2 an XbaI endonuclease site (doubly underlined) preceded by two adenine residues replaced the complete left arm. Solid lines mark the inverted repeats. Panel B shows the effects of mutations on in vivo lacZ expression during growth using WT (▪), Opp15 (a) (×) and Opp15 (b) (•) lacZ fusion constructs. Activity is depicted as the change of fluorescence per unit time as function of the optical density.

FIG. 4D: Promoter activity of PoppD variants. L. lactis LL108 strains carrying the lacZ reporter plasmids were grown in GM17 medium. Cells were harvested in the exponential phase of growth and β-galactosidase activity was measured (solid bars). The experiments were carried out in triplicate. Error bars indicate standard deviations. Open bars indicate a quantitative representation of the DNA binding assay shown in panel C. The relative binding affinity of H6-CodY for the PoppD variants was calculated by comparing the intensity of the shifted, H6-CodY bound complexes with the total radioactive signal in each lane in the presence of 100 ng protein.

FIG. 5 H6-CodY binding to fragments of derepressed variants of lacZ fusion constructs.

Panel A shows the positions of the basepair substitutions (indicated in bold) of the mutants that showed distorted repression by CodY relative to the WT. Solid lines mark the inverted repeat. Panel B shows the relative binding of H6-CodY (compared to WT) to labelled PCR products encompassing the promoter region of the mutants in an in vitro binding assay. The relative affinity was calculated by comparing the amount of H6-CodY required to shift 50% of the labelled DNA in the binding assay.

FIG. 6A Weight matrices as deduced from the seperate motifs as observed in L. lactis intergenic (upper part) and B. subtilis (lower part). IUPAC codes of the derived consensus are indicated in bold.

FIG. 6B Weight matrix obtained by pattern recognition program approach.

FIG. 7 Several conserved motifs that can be discerned in the upstream regions of CodY-regulated genes. Motif 10 represents the consensus CodY target sequence as depicted in FIG. 6.

FIG. 8 Comparison of the prtP/prtM promoter regions of L. lactis strains Wg2, SK11, E8 and BGMN1-5. The regions between bp 201 and 296 are shown double stranded for each strain, the other (flanking) regions are single stranded. Differences in the nucleotide sequences between the strains are depicted in lower case. prtP and prtM start codons are indicated in bold and with a small arrow. The deletion in the sequence of L. lactis Wg2 is indicated by a dashed line. The major transcription start sites for prtP (*) and prtM (•) are indicated above and below the sequence, respectively. Minor sites are indicated in bold face. Putative ribosomal binding sites (RBS^{P}, RBS^{M}) and −10 (−10^{P}, −10^{M}) and −35 (−35^{P}, −35^{M}) promoter sequences are overlined. Dashed arrows represent inverted repeats overlapping the promoters.

FIG. 9 Binding of H6-CodY to the prtP/prtM intergenic regions of L. lactis strains BGMN1-5, E8, SK11 and Wg2. The respective labelled DNA fragments were incubated with increasing amounts of CodY protein (as indicated above the gels) and subjected to gel electrophoresis. The positions of single stranded DNA (ss DNA) and free probe are indicated in the left margin.

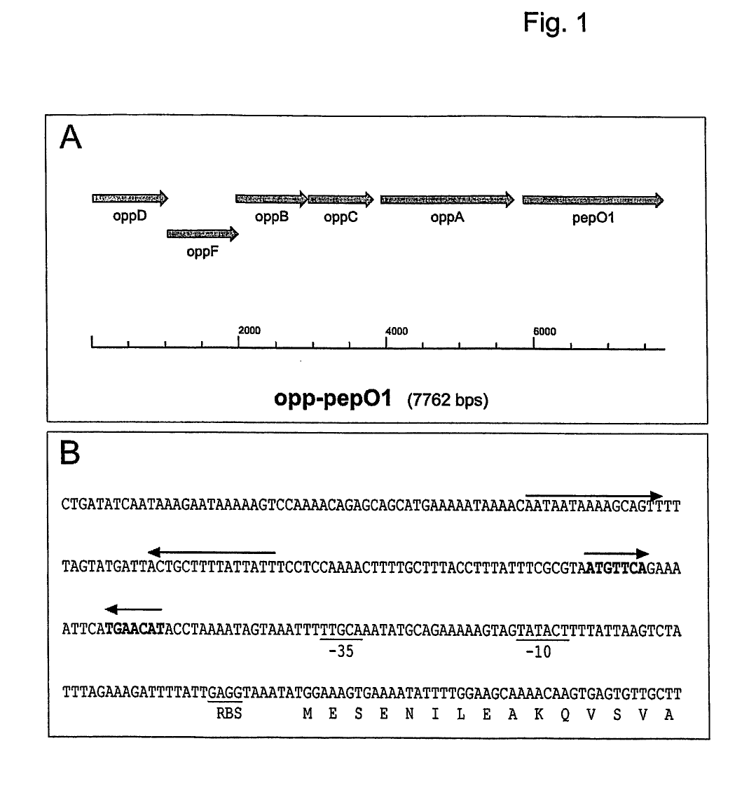

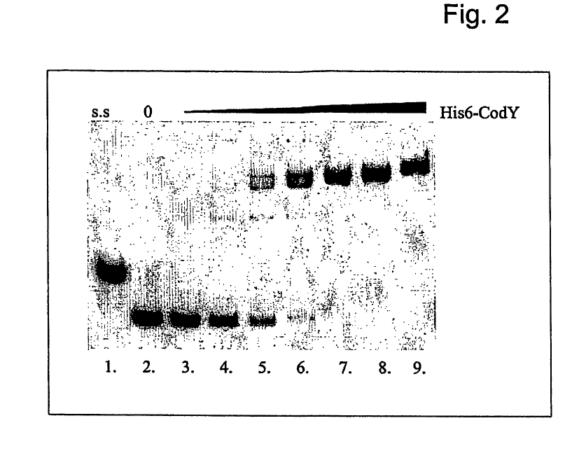

FIG. 10 . DNaseI footprinting analysis of H6-CodY binding to the oppD promoter region. The left and right panels show the footprint of the upper and lower strand, respectively. Footprints using radioactively labeled probe 3, obtained in the absence or presence of 25 or 100 ng of H6-CodY, are flanked by a Maxam and Gilbert A+G sequence ladder (AG) on the left. Numbers on the left indicate basepair positions relative to the transcriptional start site. Protected regions are marked with bars and horizontal arrows indicate the positions of hypersensitive bonds. The vertical arrows indicate the region of dyad symmetry closest to the −35 sequence (see FIG. 1B).

FIG. 11 Detection of cross-linked H6-CodY multimers. Samples containing H6-CodY were incubated at 30° C. in the presence (marked by an F) or absence of 1% formaldehyde. GMP was added to the samples in lanes 4 and 5; GTP was added to samples in lanes 6 and 7. Lane 1 contains a molecular weight marker of which the sizes, in kilodaltons, are indicated in the left margin. The positions of monomeric, dimeric and tetrameric forms of H6-CodY are indicated in the right margin by closed, open and dotted arrows, respectively.

FIG. 12 EMSA analysis of the effects of BCAAs and GTP on H6-CodY binding to PoppD. Radioactively labeled probe 3 (see FIG. 3A) was incubated alone or in the presence of 50 ng of purified H6-CodY (marked by − or + signs, respectively). Where indicated, samples were incubated in the presence of 0.6 or 2.5 mM of Val, Leu, Ile and GTP, respectively. The positions of free probe, single stranded DNA and CodY-probe complexes are indicated in the right margin.

FIG. 13 Binding of H6-CodY to radioactively labeled DNA probes spanning the oppD, asnB promoter and ctrA upstream region.

TABLE 1

Comparison of putative GTP binding motifs
in CodY homologs

| | G1 | G3 | G4 |
|---|---|---|---|
| Small GTPases | GXXXXGXT | DXXG | NKXD |
| Consensus sequences | A S | TQ | |
| FtsZ | LGGGTGTG | DAFG | TSLD |
| E. coli | | | |
| CodY | GGERLGTL | DRVG | NKFL |
| B. subtilis | | | |
| B. stearothermophilus | GGERLGTL | DRVG | DKFL |
| B. halodurans | GGQRLGTL | DRVG | DKFL |
| B. anthracis | GGERLGTL | NAa | NAa |
| C. difficile | GGMRLGSL | DRIG | NEGI |
| C. acetobutylicum | NRERLGTL | DRVG | ILND |
| S. pneumoniae | SGIRLGSL | DRIG | LISD |
| E. faecalis | AGKRLGTI | DRVG | NQQF |
| S. mutans | GGMRLGSL | DRIG | NEGI |
| S. aureus | GGERLGTL | DRIG | EKGI |
| S. pyogenes | GGMRLGSL | DRIG | NEGI |
| L. lactis | SGMRLGTF | DKIG | TGLF |

^{a}(uit Ratnayake-Lecamwasam et al., 2001), NA; not available.

TABLE 2
Genes up in CodY strain

| Genes | | |
|---|---|---|
| up in | | |
| dcodY | Fold | Annotation |
| optS | 8.8 | oligopeptide ABC transporter substrate binding protein |
| ctrA | 6.3 | cationic amino acid transporter |
| pepO | 5.7 | neutral endopeptidase |
| citB | 5.6 | aconitate hydratase (EC 4.2.1.3) |
| oppA | 5.3 | oligopeptide ABC trasporter substrate binding protein |
| gltD | 5.2 | glutamate synthase small subunit (EC 1.4.1.13) |
| ilvD | 4.8 | dihydroxy-acid dehydratase (EC 4.2.1.9) |
| ilvN | 4.5 | acetolactate synthase small subunit (EC 4.1.3.18) |
| asnB | 4.4 | asparagine synthetase B |
| ymdC | 3.9 | kanamycin kinase (EC 2.7.1.95) |
| hisA | 3.8 | phosphoribosylformimino-5-aminoimidazole isomerase |
| oppB | 3.4 | oligopeptide ABC trasporter permease protein |
| oppC | 2.9 | oligopeptide ABC trasporter permease protein |
| oppF | 2.8 | oligopeptide ABC trasporter ATP binding protein |
| llrH | 2.7 | two-component system regulator |
| hisB | 2.7 | imidazoleglycerol-phosphate dehydratase (EC 4.2.1.19) |
| serC | 2.6 | phosphoserine aminotransferase (EC 2.6.1.52) |
| hisK | 2.5 | histidinol phosphatase |
| oppD | 2.5 | oligopeptide ABC trasporter ATP binding protein |
| yahD | 2.4 | HYPOTHETICAL PROTEIN |
| leuC | 2.3 | 3-isopropylmalate dehydratase large subunit |
| | | (EC 4.2.1.33) |
| optD | 2.3 | oligopeptide ABC trasporter ATP binding protein |
| serB | 2.2 | phosphoserine phosphatase (EC 3.1.3.3) |
| hisH | 2.1 | amidotransferase (EC 2.4.2.—) |
| udp | 2.1 | uridine phosphorylase |
| aldB | 2.0 | alpha-acetolactate decarboxylase (EC 4.1.1.5) |
| hisI | 2.0 | phosphoribosyl-ATP pyrophosphohydrolase |
| | | (EC 3.6.1.31) |
| icd | 1.9 | isocitrate dehydrogenase (EC 1.1.1.42) |
| yafC | 1.9 | HYPOTHETICAL PROTEIN |
| arcD1 | 1.8 | arginine/ornitine antiporter |
| ilvB | 1.8 | acetolactate synthase large subunit (EC 4.1.3.18) |
| optF | 1.8 | oligopeptide ABC trasporter ATP binding protein |
| lacR | 1.8 | lactose transport regulator |
| hisD | 1.7 | histidinol dehydrogenase (EC 1.1.1.23) |
| arcC2 | 1.7 | carbamate kinase (EC 2.7.2.2) |
| optA | 1.7 | oligopeptide ABC transporter substrate binding protein |
| serA | 1.7 | D-3-phosphoglycerate dehydrogenase (EC 1.1.1.95) |
| recD | 1.7 | exodeoxyribonuclease V alpha chain (EC 3.1.11.5) |
| ilvC | 1.7 | ketol-acid reductoisomerase (EC 1.1.1.86) |
| ywaD | 1.7 | UNKNOWN PROTEIN |
| ydbD | 1.7 | UNKNOWN PROTEIN |
| glgD | 1.7 | glucose-1-phosphate adenylyltransferase (EC 2.7.7.27) |
| lmrP | 1.6 | integral membrane protein LmrP |
| pdhC | 1.6 | component of PDH complex (EC 2.3.1.12) |
| mesJ | 1.6 | cell cycle protein MesJ |
| lcnD | 1.6 | lactococcin A ABC transporter permease protein |
| glgA | 1.6 | glycogen synthase (EC 2.4.1.21) |
| trxA | 1.6 | thioredoxin |
| gltA | 1.6 | citrate synthase (EC 4.1.3.7) |
| pepN | 1.6 | aminopeptidase N |
| arcA | 1.6 | arginine deiminase (EC 3.5.3.6) |
| glgC | 1.6 | glucose-1-phosphate adenylyltransferase (EC 2.7.7.27) |
| yohC | 1.6 | transcriptional regulator |
| ywiE | 1.6 | UNKNOWN PROTEIN |
| yohD | 1.6 | UNKNOWN PROTEIN |
| yfiD | 1.5 | UNKNOWN PROTEIN |
| ycaF | 1.5 | UNKNOWN PROTEIN |
| ybhE | 1.5 | HYPOTHETICAL PROTEIN |
| rmlA | 1.5 | glucose-1-phosphate thymidylyltransferase (EC 2.7.7.24) |
| grpE | 1.5 | stress responce protein GrpE |
| yccE | 1.5 | UNKNOWN PROTEIN |
| hrcA | 1.5 | heat-inducible transcription repressor HrcA |
| pi336 | 1.5 | prophage pi3 protein 36 |
| dhaM | 1.5 | dihydroxyacetone kinase (EC 2.7.1.2) |
| ywjC | 1.5 | UNKNOWN PROTEIN |
| purC | 1.5 | phosphoribosylaminoimidazole synthetase |
| xynB | 1.5 | beta-1,4-xylosidase (EC 3.2.1.37) |
| lysA | 1.5 | diaminopimelate decarboxylase (EC 4.1.1.20) |
| yccF | 1.5 | HYPOTHETICAL PROTEIN |
| rmaB | 1.4 | transcriptional regulator |
| optC | 1.4 | oligopeptide ABC trasporter permease protein |
| araT | 1.4 | aromatic amino acid specific aminotransferase |
| yciA | 1.4 | amino acid amidohydrolase |
| pi235 | 1.4 | prophage pi2 protein 35 |
| yfgC | 1.4 | UNKNOWN PROTEIN |
| pepC | 1.4 | aminopeptidase C |
| yaiB | 1.4 | HYPOTHETICAL PROTEIN |
| ybhD | 1.4 | UNKNOWN PROTEIN |
| ribA | 1.4 | GTP cyclohydrolase II (EC 3.5.4.25) |
| pyrG | 1.4 | CTP synthetase |
| ps120 | 1.4 | prophage ps1 protein 20 |
| yidA | 1.4 | transcription regulator |
| yahC | 1.4 | UNKNOWN PROTEIN |
| ps305 | 1.4 | prophage ps3 protein 05 |
| yndG | 1.4 | metal ABC transporter substrate binding protein |
| yahG | 1.4 | ABC transporter ATP binding protein |
| uxuA | 1.4 | D-mannonate dehydratase (EC 4.2.1.8) |
| ps112 | 1.4 | prophage ps1 protein 12 |
| hisC | 1.4 | histidinol-phosphate aminotransferase (EC 2.6.1.9) |
| arcC1 | 1.4 | carbamate kinase (EC 2.7.2.2) |
| xy1X | 1.4 | acetyltransferase HYPOTHETICAL PROTEIN |
| rodA | 1.4 | rod-shape determining protein |
| yafB | 1.4 | sulfate transporter |
| ps113 | 1.4 | prophage ps1 protein 13 |
| ruvA | 1.4 | DNA helicase RuvA |
| asd | 1.4 | aspartate-semialdehyde dehydrogenase (EC 1.2.1.11) |
| sugE | 1.4 | SugE protein |
| groES | 1.4 | 10 KD chaperonin |
| rpsO | 1.4 | 30S ribosomal protein S15 |
| ybdA | 1.4 | transcription regulator |
| bcaT | 1.4 | branched-chain amino acid aminotransferase |
| | | (EC 2.6.1.42) |
| ybiK | 1.4 | UNKNOWN PROTEIN |
| yafJ | 1.4 | HYPOTHETICAL PROTEIN |
| pi302 | 1.4 | prophage pi3 protein 02 |
| aldR | 1.4 | regulatory protein AldR |
| menX | 1.4 | protein in menaquinone biosynthesis pathway |
| dapB | 1.4 | dihydrodipicolinate reductase (EC 1.3.1.26) |
| yphI | 1.4 | UNKNOWN PROTEIN |
| ydcG | 1.3 | transcriptional regulator |
| amtB | 1.3 | ammonium transporter |

TABLE 3
Genes down in CodY strain

| Genes | | |
|---|---|---|
| down in | | |
| dcodY | Fold | Annotation |
| plpA | 3.3 | outer membrane lipoprotein precursor |
| cysK | 3.2 | cysteine synthase (EC 4.2.99.8) |
| metB2 | 2.9 | cystathionine gamma-synthase (EC 4.2.99.9) |
| plpB | 2.9 | outer membrane lipoprotein precursor |
| cysD | 2.6 | O-acetylhomoserine sulfhydrylase |
| plpC | 2.5 | outer membrane lipoprotein precursor |
| plpD | 2.3 | outer membrane lipoprotein precursor |
| yndE | 2.3 | UNKNOWN PROTEIN |
| cysM | 2.1 | cysteine synthase (EC 4.2.99.8) |
| yrbB | 2.0 | HYPOTHETICAL PROTEIN |
| yjgC | 2.0 | amino acid ABC transporter substrate binding protein |
| argH | 1.8 | argininosuccinate lyase (EC 4.3.2.1) |
| lysP | 1.8 | lysine specific permease |
| gntK | 1.8 | gluconate kinase (EC 2.7.1.12) |
| codY | 1.7 | transcriptional regulator |
| yshA | 1.7 | amino acid permease |
| ymgI | 1.6 | UNKNOWN PROTEIN |
| ytaA | 1.6 | conserved hypopthetical protein |
| panE | 1.6 | ketopantoate reductase (EC 1.1.1.169) |
| yhcE | 1.6 | conserved hypothetical protein |
| ywdD | 1.5 | UNKNOWN PROTEIN |
| ymbC | 1.5 | UNKNOWN PROTEIN |
| rpmD | 1.5 | 50S ribosomal protein L30 |
| ywjA | 1.5 | UNKNOWN PROTEIN |
| ynfG | 1.5 | HYPOTHETICAL PROTEIN |
| ybiD | 1.5 | HYPOTHETICAL PROTEIN |
| ydcB | 1.5 | amino acid ABC transporter ATP binding protein |
| ypjC | 1.5 | UNKNOWN PROTEIN |
| ytjE | 1.5 | aminotransferase |
| pydA | 1.5 | dihydroorotate dehydrogenase A (EC 1.3.3.1) |
| ymgG | 1.5 | HYPOTHETICAL PROTEIN |
| nrdG | 1.4 | ribonucleoside-triphosphate reductase activating protein |
| yxeA | 1.4 | HYPOTHETICAL PROTEIN |
| yueD | 1.4 | conserved hypothetical protein |
| ymbK | 1.4 | UNKNOWN PROTEIN |
| ylfH | 1.4 | N-acetylglucosamine catabolic protein |
| yqcA | 1.4 | UNKNOWN PROTEIN |
| yudI | 1.4 | HYPOTHETICAL PROTEIN |
| yvdF | 1.4 | amino acid ABC transporter substrate binding protein |
| yxaF | 1.4 | HYPOTHETICAL PROTEIN |
| ymbJ | 1.4 | UNKNOWN PROTEIN |
| ynfH | 1.4 | UNKNOWN PROTEIN |
| yxbE | 1.4 | conserved hypothetical protein |
| dxsA | 1.4 | 1-deoxyxylulose-5-phosphate synthase |
| murI | 1.4 | glutamate racemase (EC 5.1.1.3) |
| ytbC | 1.4 | UNKNOWN PROTEIN |
| yccB | 1.4 | UNKNOWN PROTEIN |
| yhcH | 1.4 | HYPOTHETICAL PROTEIN |

TABLE 4

Whole genome search for the presence of the
putative CodY box in intergenic regions of
L. lactis IL1403.

| | | |
|---|---|---|
| Sequence | Gene | Despription |

| | | |
|---|---|---|
| TAATTTTCTGATAATATAGTCAATTT | yreE | UNKNOWN PROTEIN |
| TAATTTACTGACAAGTCTGTCAGTAA | ctrA | cationic amino |
| | | acid transporter |
| TAATTTACTGACAAAATTATCAGAAC | yciC | HYPOTETICAL |
| | | PROTEIN |
| AAATTTTCTGACAATAATAAAAATTG | optA | oligopeptide ABC |
| | | transporter sub- |
| | | strate binding |
| | | protein |
| AAATTATCAGAAAAATACAACAATAT | optS | oligopeptide ABC |
| | | transporter sub- |
| | | strate binding |
| | | protein |
| TAATTTTCAGAATAATATGAAAATTC | optS | oligopeptide ABC |
| | | transporter sub- |
| | | strate binding |
| | | protein |
| TAATTTACTGATAGATTTGTCAGTAA | parA | chromosome |
| | | partitioning |
| | | protein |
| TAATTTACTGACAGTTCTGTCAGTAA | aroF | Tyr-sensitive |
| | | aldolase |
| AAATTTACTGACAAAAAAGATAATGG | vacB1 | RIBONUCLEASE II |
| | | (RNB) FAMILY |
| TAATTTTCAGAAAACATAACCATTAT | optA | oligopeptide ABC |
| | | transporter sub- |
| | | strate binding |
| | | protein |
| GAATTTTATGAAAAAAATATTAATTG | ypaG | HYPOTHETICAL |
| | | PROTEIN |
| GAATTTACTGACGAATCTATCATTAA | yiaB | oxidoreductase |
| TCATTCTCTGACAAATCTGTCAGTAA | ysdC | HYPOTHETICAL |
| | | PROTEIN |
| AAATTTACTGACAAGCTTGTTAGTAT | hemK | protoporphyrinogen |
| | | oxidase |
| AAATTTAATGATAAAACAATTAGTTT | prfC | peptide chain |
| | | release factor 3 |
| AAAGTTACTGACAAATCTGTCAGTAA | yugB | ORF |
| TTATTTACTGACAAGTCTGTCAGTAA | murD | PEPTIDOGLYCAN |
| | | BIOSYNTHESIS |
| TATTTTACTGACAAAAAAATAAGTTT | ywdG | HYPOTHETICAL |
| | | PROTEIN |
| TAATTTACTGACAGCTTTGTCAGTAA | parC | topoisomerase IV |
| | | subunit B |
| | | (EC 5.99.1.-) |
| AAATTTACTGACAGAGCTGTCAGTAA | pepC | aminopeptidase |
| AAATTTACTGACAGACTTGTTAGTAA | mutM | formamidopyrimi- |
| | | dine-DNA glyco- |
| | | sylase |
| | | (EC 3.2.2.23) |
| AAATTTACTGACAACTTTGTCAGAAG | rgpAB | rhamnosyltrans- |
| | | ferase |
| AAAATGTCTGATAAAATGATTAATAC | vacB1 | RIBONUCLEASE II |
| | | (RNB) FAMILY |
| TAATTTACTGACAGAATTTTAAATTT | recN | DNA repair protein |
| AAAATTACTAACAAAACTGTTAGTAA | llrH | two-component |
| | | system regulator |

Detected motifs are sorted according to their similarity to the consensus

TABLE 4A
Search of motif in L. lactis MG1363 genome

| | | | |
|---|---|---|---|
| Position | Sequence | Score | Gene |

| | | | |
|---|---|---|---|
| 352819 | AATTTTCAGAAAATT | 11.8 | dppP |
| 164084 | AATTGTCAGAAAATT | 11.3 | codY |
| 558564 | AATTTTCTGATAATT | 11.3 | serC |
| 1799629 | AATTTTCAGATAATT | 11.3 | thrA |
| 1860656 | ATTTTTCAGAAAATT | 11.3 | ygjD |
| 2150244 | AATTTTCGGAAAAAT | 11.3 | ytjF |
| 628910 | AATTTTCAGAAAATA | 11.2 | gitA |
| 191904 | ACTTTTCAGAAAATT | 11.1 | feoA |
| 636386 | AATTTTCTGAATATT | 11.1 | yghD |
| 1516191 | ATTTTTCAGAAAAAT | 11.1 | nrdF |
| 112095 | AATTGTCAGACAATT | 10.9 | ctrA |
| 351027 | ATTTTTCTGACAATT | 10.9 | dppA |
| 683034 | AATTTTCTGAACATT | 10.9 | oppD |
| 2150244 | ATTTTTCCGAAAATT | 10.9 | ytjF |
| 279800 | TATTTTCAAAAAATT | 10.8 | ychG |
| 351095 | ATTATTCTGAAAATT | 10.8 | dppA |
| 383134 | TATTTTCAAAAAATT | 10.8 | lysS |
| 1474895 | AATTTTTTGAAAATT | 10.8 | ORF |
| 1815549 | AAATTTCTGAAAATT | 10.8 | rliC |
| 353051 | AATTTTCTGACAATA | 10.7 | dppP |
| 416868 | ATTTTTCTGAAAATA | 10.7 | yeaG |
| 742665 | ATTTTTCAGAAAATA | 10.7 | pip |
| 884469 | GTTTTTCTGAAAATT | 10.7 | yqcE |
| 1809077 | AATTTTCAGAAGAAT | 10.7 | menD |
| 16128 | AATTTTCTAAAAAGT | 10.6 | mfd |
| 352983 | AATTTTCAGAAAACA | 10.6 | dppP |
| 547027 | TATTTTCAAAAAAAT | 10.6 | yfhC |
| 868871 | CATCTTCAGAAAATT | 10.6 | arsC |
| 876166 | AATATTCAGAAAATA | 10.6 | amtB |
| 939654 | AATTTTCAAAAAACT | 10.6 | purL |
| 1001446 | AATTTTCAGAAAAGA | 10.6 | yojB |
| 1251475 | AATGTTCTGACAAAT | 10.6 | ilvD |
| 1920664 | AATTTTTTGAAAAAT | 10.6 | ysbD |
| 1940975 | AATTTTTTGAAAAAT | 10.6 | ysdE |
| 2105722 | AATTTTCTAAAAACT | 10.6 | ORF |
| 100225 | ATTTTACAGAAAATT | 10.5 | ybaB |
| 126604 | AATTATCAGAAAATG | 10.5 | ORF |
| 419279 | AATTATCAGAAAATC | 10.5 | yebA |
| 479710 | ATTTTTCTGAAAATG | 10.5 | ORF |
| 501762 | AATTTTCTGACGATT | 10.5 | cysM |

TABLE 5

Presence of putative CodY box upstream of
CodY regulated genes identified by Molle
et al. 2003.

| | |
|---|---|
| gene | sequence |

| | |
|---|---|
| yufN | ATTATCAGAAAATTT |
| citB | ATTGTGAGAAAATTG |
| dppA | TTTGTTAGAATATTC |
| hutP | GTTATCAGAATTTTT |
| yxbC | ATTATCAGAGGATTA |
| yurPQ | AATTTCAGAAAATAA |
| ycgM | ATTTTGAGGATATTG |
| yhjC | AATTTCAGACAATTC |
| ybgE | ATATTCTGAAATTTA |
| ykbA | TTTATCAAAAAAGTC |
| ggaA | ATTTTCAGCAAAAAA |
| ycgM | ATAATCAGAATCTTT |
| yoaD | TTTTTATGAAAAATA |
| guaB | GTTATCTAAATATTT |
| ilvD | ATTGTCAAAATAAAA |
| yxbBC | ATTGACAGAATTATC |
| rocA | TTTTTCAGCAAAAGA |
| yhdG | TTTTTCTAACAATTT |
| yusC | TTTTGCAGAAAAAAC |
| yusC | TTTCTTAGAATAGTG |

Detected motifs are sorted according to their similarity to the consensus

TABLE 6

Whole genome search for the presence of the
putative CodY box in intergenic regions of
B. subtilis 168.

| Sequence | | |
|---|---|---|
| motif | Gene | notation |
| ATTATCAGAAAATTT | yufN | similar to ABC transporter |
| | | (lipoprotein) |
| ATTTTCAGAAATTTA | ydjJ | function = “unknown & |
| | | similar to sugar |
| | | transporter |
| TTTTTCAGAAAAATG | ytkC | similar to autolytic |
| | | amidase |
| TTTTTCAGAAAAATC | lytE | alkaline phosphatase A & |
| | | cell wall lytic activity |
| ATTGTCTGAATATTA | yoaC | similar to phosphoglycerate |
| | | dehydrogenase |
| TTTTTCTGAATATTC | ytnA | similar to proline permease |
| ATTTTCGGAAAATTT | aldY | aldehyde dehydrogenase |
| ATTTTCAGAAAAGTT | glnQ | glutamine ABC transporter |
| ATTTTCAGAAAATAA | ykuW | function = “unknown |
| ATTTTCAGTATATTT | yndN | |
| TTTATCAGAAAAATA | yheI | |
| ATTTTCAGGAAATTC | ykuM | similar to transcriptional |
| | | regulator (LysR family)” |
| ATTTTCAGAACAATT | gbsA | similar to hypothetical |
| | | proteins |
| ATTTTCAGAAAATCA | ynaC | function = “unknown |
| ATTGTCAGAAAACTT | yqiQ | similar to phosphoenolpy- |
| | | ruvate mutase |
| ATTTTCAGAATTATA | ggaA | membrane-bound protein |
| TTTTTCGGAATATTC | yurY | similar to ABC transporter |
| | | (ATP-binding protein)” |
| TTTATCTGAAAATTT | ureA | urease (beta subunit)” |
| ATATTCAGAATATTC | leuS | leucyl-tRNA synthetase |
| ATTTTCTGAAATTTC | yurO | similar to multiple sugar- |
| | | binding protein |
| ATTTTCTGAAATTTA | ykrQ | similar to hypothetical |
| | | proteins |
| TTTTTCAGTATATTT | yvcC | similar to ABC transporter |
| | | (ATP-binding protein)” |
| TTTTTCAGCAAATTT | thiC | thiamine-phosphate pyro- |
| | | phosphorylase |
| TTTTTCAGACAATTG | yvaV | similar to hypothetical |
| | | proteins |
| ATTGTCAGCATATTT | fliR | required for flagellar |
| | | formation |
| TTTTTCATAAAATTT | yokA | assimilatory nitrate |
| | | reductase |
| TTTTTCATAAAATTC | ygeY | similar to hypothetical |
| | | proteins |
| ATTGTTAGAAAATTA | yjcL | function = “unknown” |
| ACTTTCAGAATATTT | rapE | response regulator |
| | | aspartate phosphatase” |
| TTTTTCTGAATAATT | bfmBB | ranched-chain alpha-keto |
| | | acid dehydrogenase E2 |
| | | subunit |

Detected motifs are sorted according to their similarity to the consensus

TABLE 7

Whole genome search for the presence of the putative CodY
box in intergenic regions of Streptococcus pneumoniae.

| | | |
|---|---|---|
| Sequence | Gene | Description |

| | | |
|---|---|---|
| TGATTTTCAGAAAAATTTAAGAAAAA | ppmA | Proteinase maturation protein |
| ATATTTTCTGAAAATTTCTTCAGTAA | Leu-A | 2-isopropylmalate synthase, truncation |
| TTATTATCAGATAATTTTATCAATCG | hsds | type I restriction enzyme |
| CAATTTTCTGATAATTCGGTATATTC | livJ | ABC transporter branched chain amino acid |
| GAATTTTCTGAAAATTACAAAATATA | ilvE | Branched-chain-amino-acid transaminase |
| TTATTTTCTGAAAATTTGGTAAAATA | gapN | NADP-dependent glyceraldehyde-3-phosphate |
| TCATTTTTTGAAAAAATGATTATTAC | mefE | ABC transporter - macrolide efflux |
| CATTTTTCAGAAAATTCTTTTATTTC | IS1381 | Degenerate transposase |
| ACATTTTCTGAAATTAAAAATAATAT | ugd | UDP-glucose dehydrogenase |
| GAAATTTCTGAAAAATATGATATAAT | hemK | protoporphyrinogen oxidase |
| AAATTATTTGATAATTCTATAATTTC | spr1649 | Putative transcriptional regulator (phoU like) |
| AATTTATCTGAAAAAACGAAAAATAT | spr1765 | Hypothetical protein |
| CAATTTTTTGAAAAAATATTGATTTA | glgB | 1,4-alpha-glucan branching enzyme |
| TATTTTTCTGAAAACTCTGATATAAA | spr2010 | Probable member of DHH superfamily |
| TCATTTTCAGATAAGGATAAAAATTG | spr0128 | Hypothetical protein |
| GAATTGACAGATGAATTTGTTAAGAA | ABC-NDB | ABC transporter ATP-binding protein - unknown |
| AAATTGAATGAAAAGTATAAAATTAA | spr1149 | Probable oligosaccharide repeat unit transporter |
| AAATTGTCAGAATTATGAGAAAATAG | rgg/ | transcriptional regulator of glucosyltransferase |
| | spr1934 | |
| CATTTTACTGAAGAATACGATATTAT | gidA | Glucose inhibited division protein |
| AAATTTCTAGATAAGTTAATAATTAA | abc-n/p | ABC transporter - unknown substrate |
| ATATTCTGTGAAAAATAAAATAGTAT | spr0119 | Hypothetical protein |
| GAATTTCCAGATAAACTAAAAAAATC | hsdS 2X | type I restriction enzyme |
| ACATTATCTGAAAAATTAAACTATAA | spr0607 | Hypothetical protein |
| TAATTGTCAGAAAGTTAAATAAAGGA | rpmI | 50S Ribosomal protein L35 |
| AAATTATCTAATAACAAAAATATTAT | spnIM | DNA modification methyltransferase |
| TTATTTACAGAAAGAACAAAAAATGC | spnIM | DNA modification methyltransferase |
| GAATTTTCAGAAAATTCTATACGCAT | ilvD | Dihydroxyacid dehydratase |
| ATATTTTTTGAAAAATTTTTTAAAAA | arcA | Arginine deiminase, truncation |
| AAATTGACAAATAAAAATTTGAATAT | ABC-NBD | ABC transporter ATP-binding protein anion transport |
| TACTTTACAGAAGAATTACAGAATAG | mfd | Transcription-repair coupling factor |
| GAATTTTCAGAATAATCTGTATATGT | spr0157 | Conserved hypothetical protein |
| GAATTTCCTGAAAATCTGGCTATTAT | murM | Serine/alanine adding enzyme |
| GATTTTTCGGAAAATTATGTTAGAAT | zwf | Glucose-6-phosphate 1-dehydrogenase |
| CAATATTTAGAAAAAAAAGAAATTAA | licD1 | phosphorylcholine incorporation in teichoic acids |
| ATATTTTCTGAACAATTAATATATTC | spr1403 | Hypothetical protein |
| CATTTTGCGGAAAATTGAGTAAATAT | relA | GTP pyrophosphokinase |
| TAATTTTCTGATTTTTTGTAAAATAA | spr1650 | Hypothetical protein |
| TTATTTTCTAATAGATATAAAATTAT | pcpA | Choline-binding protein |
| TAAATTTCAGATTGATGAAAAAATAG | rsuA | Ribosomal small subunit pseudouridine synthase |
| AATTTTTTAGAAAAAAGTGTAATTTT | ptsG | PTS glucose-specific enzyme IIABC component |
| AAATTCACTGAAAGTTTAAATATGAC | hsdS | type I restriction enzyme |
| GAATTGCCAGACTATTTTAATACTAT | spr0803 | Hypothetical protein |
| AGATTTTAAGTAAAATTTATTAGTAA | spr0826 | Hypothetical protein |
| AAATTTTAGAAAAAATTAAAGAATAC | flaV | Flavodoxin |
| CAATTATCTGATCATCTGAAAAATAT | ABC-NP | ABC transporter - multidrug resistance |

Detected motifs are sorted according to their similarity to the consensus

TABLE 8

Whole genome search for the presence of the
putative CodY box in intergenic regions of
Streptococcus agalacticiae.

| Sequence | | |
|---|---|---|
| motif | Gene | Description |
| ATTATCAGAATATTG | gbs1632 | similar to branched-chain |
| | | amino acid ABC transporter |
| ATTTTCAGAAAAATA | gbs1105 | similar to unknown protein |
| TTTATCAGAAAATTT | gbs0662 | similar to ABC transporter |
| | | (ATP-binding protein) |
| ATTTTCTGAATATTC | gbs2002 | similar to glycerol |
| | | dehydrogenase |
| TTTTTCTGAATATTT | gbs1489 | possible surface protein |
| ATTTTCAAAAAATTT | gbs0054 | similar to alcohol |
| | | dehydrogenase |
| ATTTTCAAAAAATTG | gbs0008 | similar to unknown protein |
| ATTGTCAGAATTTTC | gbs1406 | Similar to ABC transporter |
| ATTATCTGAAAATTT | gbs0144 | similar to oligopeptide |
| | | ABC transporter |
| ATTATCTGAATATTA | gbs0898 | acetoin dehydrogenase E3 |
| ATTTTCAGTATATTC | gbs2007 | similar to C5A peptidase, |
| | | putative peptidoglycan |
| | | linked protein |
| ATTTTCAGAAAATGT | gbs0235 | gene = “rRNA-16s |
| ATTATCAGAAGATTT | gbs0577 | Similar to unknown |
| | | proteins |
| ATTTTCAGATAATTG | gbs0143 | similar to oligopeptide |
| | | ABC transporter |
| ATTTTTAGAAAATTA | gbs0604 | similar to negative regu- |
| | | lator of FtsZ ring forma- |
| | | tion protein EzrA |
| ATTTTCTGAATAATT | dnaG | DNA primase |
| ATTTTCTGAATAATT | ftsA | Similar to cell division |
| | | protein FtsA |
| ATTTTCAGGATATTT | gbs1259 | Similar to ABC transporter |

Detected motifs are sorted according to their similarity to the consensus

TABLE 9
Bacterial strains and plasmids used in this patent application

| Strain or plasmid | Relevant phenotype or genotype | Source or reference |
|---|---|---|
| Strains | | |
| L. lactis subsp. | | |
| cremoris | | |
| MG1363 | Lac⁻; Prt⁻; Plasmid-free derivative of NCDO712 | (Gasson, 1983) |
| LL108 | Cm^{r}, MG1363 derivative containing pWV01 repA | (Leenhouts et al., |
| | gene in the chromosome | 1998) |
| LL302 | RepA⁺ MG1363, carrying one copy of pWV01 repA | (Leenhouts et al., |
| | gene on the chromosome | 1998) |
| NZ9000 | MG1363 pepN::nisRK | (Kuipers et al., |
| | | 1998) |
| NZ9700 | Nisin producing transconjugant containing the | (Kuipers et al., |
| | nisin-sucrose transposon Tn5276 | 1993) |
| SK11 | Lac⁺; Prt⁺; harbours, a.o., proteinase plasmid | (de Vos et al., 1984) |
| | pSK111 | |
| Wg2 | Prt⁺; harbours, a.o., proteinase plasmid pWV05 | (Otto et al., 1982) |
| E8 | Lac⁺; Prt⁺ | (Kok, 1990) |
| L. lactis subsp. | | |
| lactis | | |
| BGMN1-5 | Wild type strain, PrtP⁺, Bac501⁺, Bac513⁺ | (Gajic et al., 1999) |
| E. coli | | |
| EC101 | Kan^{r}; JM101 with repA from pWV01 integrated | J.Law et al., 1995 |
| | in chromosome | |
| Plasmids | | |
| pNZ8048 | Cm^{r}, inducible expression vector carrying P_{nisA} | (Kuipers et al., |
| | | 1998) |
| pNH6CodY | his6-codY of L. lactis MG1363 behind P_{nisA} | Gajić, to be |
| | | submitted |
| pGKH11 | Em^{r}, Cm^{r}, contains genes for α-gal and β-gal, | (Haandrikman, |
| | controlled by P_{prtM} and P_{prtP} of Wg2, respectively | 1990) |
| pGKB11 | Em^{r}, Cm^{r}, contains genes for α-gal and β-gal, | Gajić, to be |
| | controlled by P_{prtM} and P_{prtP} of BGMN1-5, | submitted |
| | respectively | |
| pGKE11 | Em^{r}, Cm^{r}, contains genes for α-gal and β-gal, | Gajić, to be |
| | controlled by P_{prtM} and P_{prtP} of E8, respectively | submitted |
| pGKS11 | Em^{r}, Cm^{r}, contains genes for α-gal and β-gal, | Gajić, to be |
| | controlled by P_{prtM} and P_{prtP} of SK11, respectively | submitted |
| pORI13 | Em^{r}; integration vector, promoterless lacZ, Ori⁺, | J. Sanders et |
| | RepA- derivative of pWV01 | al., 1998 |
| pORIopp14 | Em^{r}; pORI13 carrying 154bp oppD promoter | Gajić, to be |
| | fragment amplified with primers opp1 and opp14 | submitted |
| pORIopp15 (a) | Em^{r}; pORI13 carrying 154bp oppD promoter | Gajić, to be |
| | fragment amplified with primers opp1 and | submitted |
| | opp 15 (a) | |
| pORIopp15 (b) | Emr^{r}; pORI13 carrying 154bp oppD promoter | Gajić, to be |
| | fragment amplified with primers opp1 and | submitted |
| | opp15 (b) | |

Em^{r}, Cm^{r} Ap^{r}, resistance to erythromycin, chloramphenicol and ampicilin, respectively. P_{nisA}—inducible nisA promoter, P_{prtP}—prtP promoter, P_{prtM}—prtM promoter, α-gal—α-galactosidase, β-gal—β-galactosidase.

TABLE 10
Differentially expressed genes found in microarray expression
analysis of L. lactis MG1363 codY vs. WT.

| | Average | |
|---|---|---|
| | regulation (fold in | |
| Gene | codY vs. WT) | Significance (p-value) |
| optS | 9.8 | 7.17E−05 |
| gltD | 7.3 | 2.18E−05 |
| citB | 7.1 | 3.76E−03 |
| asnB | 6.0 | 1.08E−05 |
| ctrA | 5.8 | 3.68E−05 |
| ilvD | 5.4 | 3.81E−05 |
| pepO | 5.0 | 1.93E−05 |
| ymdC | 4.9 | 5.29E−04 |
| hisA | 4.9 | 2.52E−06 |
| ilvN | 4.7 | 6.10E−03 |
| oppA | 4.6 | 1.13E−05 |
| icd | 3.1 | 1.25E−05 |
| oppD | 3.1 | 5.86E−05 |
| hisB | 3.0 | 1.12E−02 |
| leuD | 3.0 | 5.09E−01 |
| udp | 3.0 | 1.89E−03 |
| oppB | 2.8 | 1.69E−04 |
| yahD | 2.7 | 1.15E−01 |
| oppC | 2.7 | 1.67E−03 |
| yohC | 2.6 | 3.00E−02 |
| yveA | 2.6 | NA |
| uxuA | 2.5 | NA |
| serC | 2.5 | 2.49E−03 |
| oppF | 2.5 | 2.92E−06 |
| trxA | 2.4 | NA |
| arcC2 | 2.3 | 5.03E−03 |
| ilvC | 2.3 | 9.65E−02 |
| serA | 2.3 | 2.86E−02 |
| argG | 2.3 | 2.29E−02 |
| arcD1 | 2.3 | 1.80E−04 |
| hisK | 2.2 | 7.72E−02 |
| yxbA | 2.2 | NA |
| ydgD | 2.2 | NA |
| serB | 2.2 | 7.60E−04 |
| leuC | 2.1 | NA |
| optA | 2.1 | NA |
| yjjA | 2.1 | 4.31E−01 |
| asnS | 2.1 | 4.31E−02 |
| hisI | 2.1 | 8.82E−05 |
| yxaC | 2.1 | 9.81E−02 |
| groES | 2.1 | 2.40E−03 |
| yjhC | 2.0 | 1.75E−01 |
| rmeC | 2.0 | 1.49E−01 |
| ytdB | 2.0 | 2.31E−01 |
| ysiC | 2.0 | NA |
| yndG | 2.0 | 1.36E−01 |
| optD | 2.0 | 4.14E−02 |
| amtB | 2.0 | 1.67E−04 |
| feoB | 1.9 | NA |
| hisH | 1.9 | 2.25E−02 |
| ykcC | 1.9 | NA |
| lysA | 1.9 | 1.63E−03 |
| pepN | 1.9 | 1.29E−03 |
| glgC | 1.9 | 3.76E−02 |
| aldB | 1.9 | 5.52E−05 |
| arcC1 | 1.8 | NA |
| lcnD | 1.8 | NA |
| hrcA | 1.8 | 1.48E−03 |
| yfiG | 1.8 | 1.27E−01 |
| thrC | 1.7 | 1.30E−02 |
| ydiE | 1.7 | 2.15E−01 |
| ilvA | 1.7 | NA |
| yxdG | 1.7 | 1.09E−02 |
| ynaD | 1.7 | 1.08E−03 |
| yedE | 1.7 | 2.28E−01 |
| thrA | 1.7 | 1.86E−02 |
| rodA | 1.7 | 1.18E−01 |
| ydiG | 1.7 | 1.29E−01 |
| yfiD | 1.7 | 2.82E−01 |
| yhfE | 1.7 | 7.89E−02 |
| yahA | 1.7 | 1.30E−01 |
| ydcG | 1.7 | 1.27E−04 |
| gltA | 1.6 | 6.48E−02 |
| yviC | 1.6 | 1.51E−01 |
| ybaD | 1.6 | 1.02E−01 |
| ybjA | 1.6 | 2.65E−01 |
| hisD | 1.6 | 2.76E−01 |
| glk | 1.6 | 7.18E−02 |
| rgpE | 1.6 | 2.74E−01 |
| thrB | 1.6 | 1.25E−01 |
| clpB | 1.6 | NA |
| bcaT | 1.6 | 3.87E−03 |
| hisF | 1.6 | NA |
| yvcA | 1.6 | 3.16E−01 |
| ymgK | 1.6 | NA |
| yajH | 1.6 | 3.50E−01 |
| yccE | 1.6 | 1.49E−01 |
| yndA | 1.6 | 4.79E−04 |
| ybhE | 1.6 | 1.43E−01 |
| adhE | 1.6 | 1.53E−02 |
| grpE | 1.6 | 4.25E−02 |
| yqaB | 1.5 | 2.78E−03 |
| pgiA | 1.5 | 1.19E−01 |
| yrhH | 1.5 | NA |
| ybhC | 1.5 | 2.97E−01 |
| glgD | 1.5 | 1.57E−02 |
| yidA | 1.5 | 1.13E−01 |
| yabF | 1.5 | 1.54E−02 |
| rimM | 1.5 | 1.44E−01 |
| yacl | 1.5 | 2.36E−01 |
| asd | 1.5 | NA |
| yijE | 1.5 | 2.02E−01 |
| yccF | 1.5 | 6.44E−02 |
| agl | 1.5 | 1.28E−01 |
| optC | 1.5 | 1.91E−02 |
| malF | 1.5 | 8.17E−02 |
| yjhB | 1.5 | 4.32E−01 |
| yibG | 1.5 | 4.78E−04 |
| ribA | 1.5 | NA |
| yabE | 1.5 | 3.23E−01 |
| thiL | 1.5 | 2.06E−01 |
| ycaF | 1.5 | 7.56E−02 |
| optF | 1.5 | 5.78E−02 |
| arcC3 | 1.5 | NA |
| ymfD | 1.5 | 2.40E−01 |
| ps309 | 1.5 | 2.41E−01 |
| ycfl | 1.5 | 1.68E−01 |
| uvrC | 1.5 | 5.04E−01 |
| yphC | 1.5 | 2.67E−01 |
| ybeM | 1.5 | 2.42E−01 |
| ygfA | 1.5 | NA |
| yphJ | 1.5 | 1.00E−01 |
| ynaA | 1.5 | 9.20E−02 |
| ahpF | 1.5 | 1.75E−01 |
| aroB | 1.5 | 1.47E−01 |
| ybhD | 1.5 | 1.85E−01 |
| hisC | 1.5 | 6.57E−02 |
| yoaF | 1.5 | 3.16E−01 |
| dexC | 1.5 | 2.59E−02 |
| amyY | 1.5 | 3.85E−02 |
| yueF | 1.5 | 5.47E−01 |
| ydgH | 1.5 | 4.84E−01 |
| dapB | 1.4 | 1.11E−03 |
| ftsZ | 1.4 | 1.35E−01 |
| ynhA | 1.4 | 1.07E−02 |
| ygiJ | 1.4 | 2.11E−01 |
| ptcA | 1.4 | 1.29E−01 |
| yqeH | 1.4 | 2.45E−02 |
| yrgl | 1.4 | 6.06E−01 |
| pepC | 1.4 | 2.97E−03 |
| ycfH | 1.4 | 4.66E−01 |
| ybcH | 1.4 | 4.45E−02 |
| pi345 | 1.4 | 3.31E−01 |
| ps112 | 1.4 | 5.32E−01 |
| yteD | 1.4 | NA |
| ythB | 1.4 | 9.95E−02 |
| yneD | 1.4 | 7.17E−02 |
| pmsR | 1.4 | 7.19E−02 |
| rgpC | 1.4 | 1.34E−01 |
| yjbB | 1.4 | 3.70E−01 |
| ygdF | 1.4 | 2.48E−01 |
| yoaB | 1.4 | 1.38E−01 |
| yfhK | 1.4 | 1.10E−02 |
| yvaB | 1.4 | 4.18E−01 |
| yqjA | 1.4 | 1.45E−01 |
| pi120 | 1.4 | 1.27E−03 |
| yliC | 1.4 | 6.76E−02 |
| ydgC | 1.4 | 5.49E−04 |
| rarA | 1.4 | 1.18E−01 |
| ywdG | 1.4 | 1.11E−01 |
| yfdD | 1.4 | 2.09E−01 |
| ftsW1 | 1.4 | 6.18E−03 |
| ybeB | 1.4 | 7.74E−02 |
| yvdD | 1.4 | 9.09E−03 |
| ymeB | 1.4 | 2.19E−02 |
| yoiC | 1.4 | NA |
| yhcK | 1.4 | 3.80E−01 |
| groEL | 1.4 | NA |
| xylM | 1.4 | 1.38E−01 |
| ydgG | 1.4 | 3.77E−01 |
| sugE | 1.4 | 1.86E−01 |
| pi360 | 1.4 | 3.37E−01 |
| yfhB | 1.4 | 2.97E−01 |
| metS | 1.4 | 6.97E−02 |
| yeaC | 1.4 | NA |
| coiA | 1.4 | 1.76E−01 |
| butA | 1.4 | 5.62E−03 |
| clpP | 1.3 | 1.82E−01 |
| arcA | 1.3 | 1.20E−01 |
| yacB | 1.3 | 4.34E−02 |
| kinC | 1.3 | 2.34E−02 |
| ps305 | 1.3 | 2.30E−01 |
| glnB | 1.3 | 2.92E−01 |
| ybbC | 1.3 | 3.99E−02 |
| ykhJ | 1.3 | 8.11E−02 |
| dnaK | 1.3 | 9.17E−02 |
| dnaG | 1.3 | 2.16E−03 |
| ybdJ | 1.3 | 3.38E−01 |
| yohD | 1.3 | 1.39E−04 |
| pi130 | 1.3 | NA |
| yugC | 1.3 | 1.75E−03 |
| ycjH | 1.3 | 3.25E−01 |
| yjaB | 1.3 | 1.33E−01 |
| glgP | 1.3 | 7.93E−02 |
| pi235 | 1.3 | NA |
| acmB | 1.3 | 1.78E−01 |
| xylX | 1.3 | NA |
| ybeH | 1.3 | 1.00E−01 |
| arcD2 | 1.3 | 1.82E−03 |
| rgpB | 1.3 | 6.26E−02 |
| asnH | 1.3 | 2.29E−01 |
| yedF | 1.3 | 5.47E−02 |
| accB | 1.3 | 5.35E−02 |
| fhuD | 1.3 | 5.56E−02 |
| yccG | 1.3 | 1.56E−01 |
| ypiH | 1.3 | 2.11E−01 |
| yacC | 1.3 | 2.26E−01 |
| ybeF | 1.3 | 2.13E−01 |
| ygaE | 1.3 | 3.66E−01 |
| ygaF | 1.3 | 1.38E−01 |
| ybeD | 1.3 | 2.18E−01 |
| pi302 | 1.3 | NA |
| ps302 | 1.3 | NA |
| ydgB | 1.3 | 1.14E−03 |
| hemK | 1.3 | 6.48E−02 |
| ychH | 1.3 | 2.07E−03 |
| yjhD | 1.3 | 6.68E−03 |
| yfjA | 1.3 | 2.97E−02 |
| ykhD | 1.3 | 1.11E−02 |
| accC | 1.3 | 1.54E−01 |
| ynaC | 1.3 | 1.95E−02 |
| cdsA | 1.3 | 1.94E−01 |
| mtsA | 1.3 | 2.11E−02 |
| murA2 | 1.3 | 1.46E−01 |
| ps120 | 1.3 | 8.81E−02 |
| yejJ | 1.3 | 1.31E−01 |
| ybhB | 1.3 | 2.25E−01 |
| ysaA | 1.3 | 3.07E−01 |
| yagA | 1.3 | 2.96E−01 |
| pi336 | 1.3 | 1.09E−01 |
| yjjD | 1.3 | 3.11E−01 |
| yjcA | 1.3 | 1.25E−02 |
| llrA | 1.3 | 2.06E−01 |
| msmK | 1.3 | 2.57E−01 |
| ybdA | 1.3 | 3.20E−01 |
| yjhF | 1.3 | 5.21E−04 |
| yrbC | 1.3 | 3.38E−01 |
| yhfF | 1.3 | 1.43E−02 |
| yjdE | 1.3 | 1.95E−02 |
| yhcA | 1.3 | 1.56E−01 |
| yjiB | 1.3 | 1.68E−01 |
| yjjC | 1.3 | 3.30E−02 |
| ycfG | 1.3 | 2.08E−01 |
| ycdF | 1.3 | 4.78E−01 |
| ymhC | 1.3 | 4.73E−01 |
| yjcF | 1.3 | 2.48E−01 |
| ps122 | 1.3 | 4.37E−02 |
| ywcC | 1.3 | 3.77E−01 |
| xylT | 1.3 | 8.99E−02 |
| araT | 1.3 | 1.20E−02 |
| ycjG | 1.3 | 5.87E−02 |
| yjgF | 1.3 | 3.49E−01 |
| clpE | 1.3 | 2.05E−01 |
| frdC | 1.3 | 5.17E−03 |
| gpdA | 1.3 | 9.30E−02 |
| ychD | 1.3 | 1.91E−01 |
| yniJ | 1.3 | 1.91E−02 |
| glnP | 1.3 | 1.65E−01 |
| butB | 1.3 | 1.31E−01 |
| pyrG | 1.3 | 2.66E−01 |
| ftsW2 | 1.3 | 1.21E−01 |
| yhhC | 1.3 | NA |
| noxD | 1.3 | 5.88E−03 |
| dinP | 1.3 | NA |
| recQ | −1.3 | 1.59E−01 |
| lplL | −1.3 | 9.88E−03 |
| xynD | −1.3 | 1.04E−01 |
| pi225 | −1.3 | 3.54E−01 |
| yjfG | −1.3 | 4.16E−02 |
| rheB | −1.3 | 2.17E−01 |
| pi140 | −1.3 | 2.73E−01 |
| rpmGC | −1.3 | 1.58E−01 |
| yxbC | −1.3 | 1.51E−02 |
| bglR | −1.3 | 2.45E−01 |
| yqbl | −1.3 | 5.59E−01 |
| yghB | −1.3 | 6.78E−02 |
| yqcA | −1.3 | 4.10E−01 |
| ynjE | −1.3 | 1.85E−02 |
| ymbK | −1.3 | 1.67E−01 |
| ysjD | −1.3 | 3.18E−01 |
| yweC | −1.3 | 2.43E−02 |
| galM | −1.3 | 3.24E−01 |
| ywfC | −1.3 | 2.66E−01 |
| ps201 | −1.3 | 3.98E−01 |
| pi216 | −1.3 | 3.19E−01 |
| ykbC | −1.3 | 1.31E−01 |
| yeeB | −1.3 | 9.29E−03 |
| ileS | −1.3 | 2.32E−01 |
| yjfB | −1.3 | NA |
| yxeA | −1.3 | 2.88E−03 |
| yciF | −1.3 | 1.57E−02 |
| dukA | −1.3 | 1.34E−02 |
| ywjC | −1.3 | NA |
| yvdA | −1.3 | 8.07E−02 |
| alaS | −1.3 | 1.01E−01 |
| ndrH | −1.3 | 3.45E−03 |
| gadR | −1.3 | 2.05E−01 |
| yohH | −1.3 | 8.77E−02 |
| ps119 | −1.3 | 1.74E−01 |
| ycgJ | −1.3 | 3.16E−02 |
| pi223 | −1.3 | 1.94E−01 |
| yveD | −1.3 | 3.95E−01 |
| sunL | −1.3 | 4.87E−01 |
| yjbE | −1.3 | 1.40E−02 |
| ywbB | −1.3 | 5.42E−01 |
| yteC | −1.3 | 1.24E−01 |
| metE | −1.3 | 2.85E−01 |
| ycbD | −1.3 | 5.14E−03 |
| purN | −1.3 | 2.65E−01 |
| pabB | −1.3 | 6.63E−02 |
| yrgF | −1.3 | 1.22E−01 |
| ycjM | −1.3 | 1.23E−01 |
| yljC | −1.3 | 4.16E−01 |
| yjeF | −1.3 | 1.21E−01 |
| comGC | −1.3 | 2.23E−01 |
| yvel | −1.3 | 6.03E−02 |
| ylcD | −1.3 | 3.25E−01 |
| dltD | −1.3 | NA |
| pi343 | −1.3 | 5.49E−01 |
| yueD | −1.3 | NA |
| ppiA | −1.3 | 1.43E−01 |
| dut | −1.3 | 1.00E−01 |
| purB | −1.3 | 6.79E−03 |
| ypjC | −1.3 | 3.96E−01 |
| ytaB | −1.3 | NA |
| cbr | −1.3 | 2.49E−01 |
| rpmC | −1.3 | 3.99E−01 |
| ycgH | −1.3 | 4.57E−01 |
| ybiE | −1.3 | 2.75E−04 |
| yvdF | −1.3 | 1.24E−01 |
| rpmD | −1.3 | 2.07E−03 |
| pi124 | −1.3 | 5.76E−01 |
| pbp2B | −1.3 | 2.74E−01 |
| ypjA | −1.3 | 1.21E−01 |
| yscE | −1.3 | 4.69E−01 |
| ymbC | −1.3 | 2.22E−02 |
| birA1 | −1.4 | 1.24E−02 |
| hflX | −1.4 | 1.48E−02 |
| ffh | −1.4 | 1.60E−01 |
| yecE | −1.4 | 1.13E−01 |
| ycjl | −1.4 | 1.31E−01 |
| ps315 | −1.4 | 6.96E−02 |
| comX | −1.4 | 2.55E−01 |
| pi215 | −1.4 | 1.81E−01 |
| dcdA | −1.4 | 2.51E−02 |
| glyA | −1.4 | 1.19E−01 |
| yueA | −1.4 | 1.22E−04 |
| hsdM | −1.4 | 1.36E−01 |
| ybiD | −1.4 | 6.02E−03 |
| queA | −1.4 | 6.69E−03 |
| ywaD | −1.4 | 4.66E−01 |
| pi123 | −1.4 | NA |
| pi308 | −1.4 | 1.56E−01 |
| phnC | −1.4 | 1.79E−01 |
| ycjA | −1.4 | 3.16E−02 |
| ypjl | −1.4 | 1.99E−01 |
| ykhG | −1.4 | 4.29E−02 |
| dnaA | −1.4 | 8.81E−03 |
| phnE | −1.4 | 7.20E−03 |
| yudD | −1.4 | 5.29E−01 |
| yrfA | −1.4 | 2.43E−01 |
| yeiD | −1.4 | 2.64E−03 |
| rpmF | −1.4 | 2.56E−02 |
| ysfG | −1.4 | 2.86E−01 |
| pi249 | −1.4 | 1.60E−01 |
| yuiB | −1.4 | 3.97E−01 |
| yvaD | −1.4 | 9.44E−02 |
| recD | −1.4 | 1.42E−01 |
| yfcB | −1.4 | 1.72E−01 |
| kdgR | −1.4 | 7.74E−01 |
| ps304 | −1.4 | 4.41E−02 |
| yqfC | −1.4 | NA |
| yxbF | −1.4 | 8.14E−05 |
| citE | −1.4 | 5.93E−02 |
| yfcC | −1.4 | 2.53E−01 |
| folD | −1.4 | 9.71E−02 |
| pdhD | −1.4 | 2.62E−03 |
| ps118 | −1.4 | 1.75E−01 |
| yafF | −1.4 | 3.14E−02 |
| nagB | −1.5 | 6.51E−04 |
| ymcF | −1.5 | 5.55E−01 |
| yveB | −1.5 | NA |
| yxbD | −1.5 | 3.56E−02 |
| pi319 | −1.5 | 6.45E−02 |
| yoaD | −1.5 | 3.74E−01 |
| ywaH | −1.5 | 4.10E−01 |
| gidC | −1.5 | 9.87E−03 |
| yfbK | −1.5 | 6.66E−03 |
| yrgA | −1.5 | 2.34E−01 |
| mgtA | −1.5 | 1.06E−02 |
| ynbE | −1.5 | 2.98E−01 |
| yrbE | −1.5 | 6.75E−02 |
| ynfH | −1.5 | 3.06E−02 |
| purE | −1.5 | 4.33E−01 |
| ytjE | −1.5 | 2.12E−01 |
| ymgF | −1.5 | NA |
| gltS | −1.5 | 1.23E−01 |
| kdgK | −1.5 | 3.45E−01 |
| yccH | −1.5 | NA |
| kinB | −1.5 | 1.09E−01 |
| yuiA | −1.5 | 6.52E−02 |
| yxbE | −1.6 | 4.59E−04 |
| yceE | −1.6 | 4.59E−02 |
| yueE | −1.6 | 5.60E−03 |
| ywdB | −1.6 | 3.02E−01 |
| zitR | −1.6 | 2.11E−01 |
| ypdB | −1.6 | 1.75E−01 |
| yjgB | −1.6 | 3.73E−02 |
| argD | −1.6 | 1.74E−01 |
| ps113 | −1.6 | 2.75E−01 |
| purK | −1.6 | NA |
| yrbB | −1.6 | 4.82E−02 |
| yjgC | −1.7 | 8.92E−02 |
| ykiC | −1.7 | 2.54E−01 |
| purH | −1.7 | 1.66E−01 |
| rmaB | −1.7 | NA |
| cysD | −1.7 | 3.29E−01 |
| nrdG | −1.7 | 5.10E−03 |
| pi104 | −1.7 | 1.19E−01 |
| yscD | −1.7 | 3.63E−01 |
| gntR | −1.7 | 1.82E−01 |
| tra983L | −1.7 | 3.08E−01 |
| gntK | −1.7 | 4.13E−03 |
| dxsA | −1.7 | NA |
| yjal | −1.7 | 1.15E−01 |
| ynjB | −1.7 | NA |
| rmaC | −1.7 | 1.79E−01 |
| pi230 | −1.8 | 2.01E−01 |
| yriD | −1.8 | 2.06E−01 |
| yhcE | −1.8 | 2.70E−03 |
| yveF | −1.8 | 1.91E−01 |
| ytbC | −1.8 | 2.96E−01 |
| lmrA | −1.8 | 3.06E−01 |
| yhhA | −1.8 | NA |
| purM | −1.9 | 1.99E−01 |
| codY | −1.9 | 1.04E−02 |
| pi122 | −1.9 | NA |
| yiiD | −1.9 | 2.86E−01 |
| argH | −1.9 | 8.77E−02 |
| yhcH | −2.0 | 3.61E−01 |
| panE | −2.0 | 9.68E−03 |
| purF | −2.0 | 5.26E−03 |
| ps206 | −2.0 | NA |
| metB2 | −2.0 | 5.73E−02 |
| guaC | −2.0 | 1.40E−01 |
| cysM | −2.0 | 1.88E−02 |
| yviH | −2.1 | 1.80E−01 |
| uxaC | −2.1 | 2.96E−01 |
| gmk | −2.1 | NA |
| rpsU | −2.1 | NA |
| ycgA | −2.2 | NA |
| cysK | −2.2 | 7.17E−03 |
| yshA | −2.3 | 1.71E−03 |
| ymgH | −2.3 | 2.11E−01 |
| llrG | −2.3 | NA |
| ps111 | −2.4 | NA |
| pi353 | −2.5 | 2.00E−02 |
| yliG | −2.5 | 2.61E−01 |
| ynjJ | −2.5 | 1.82E−01 |
| yndE | −2.6 | NA |
| hpt | −2.6 | NA |
| yojC | −2.7 | NA |
| plpC | −2.7 | 5.16E−03 |
| plpD | −2.7 | 1.97E−03 |
| ywdA | −2.8 | 2.03E−01 |
| yviJ | −3.2 | NA |
| purC | −3.3 | NA |
| plpB | −3.4 | 1.53E−03 |
| purL | −3.4 | 5.25E−02 |
| plpA | −3.4 | 1.08E−03 |

(NA = not available)

### REFERENCES

Alvarez, D., Novac, O., Callejo, M., Ruiz, M. T., Price, G. B., and Zannis-Hadjopoulos, M. (2002) 14-3-3sigma is a cruciform DNA binding protein and associates in vivo with origins of DNA replication J. Cell Biochem. 87: 194-207.
Buist, G., H. Karsens, A. Nauta, D. van Sinderen, G. Venema, and J. Kok. 1997. Autolysis of Lactococcus lactis caused by induced overproduction of its major autolysin, AcmA. Appl. Environ. Microbiol 63:2722-2728.
Christensen, J. E., Dudley, E. G., Pederson, J. A. and Steele, J. L. (1999). Peptidases and amino acid catabolism in lactic acid bacteria. Antonie Van Leeuwenhoek 76, 217-246.
Detmers, F. J., Kunji, E. R., Lanfermeijer, F. C., Poolman, B. and Konings, W. N. (1998). Kinetics and specificity of peptide uptake by the oligopeptide transport system of Lactococcus lactis. Biochemistry 37, 16671-16679.
Ebbole, D. J. and Zalin, H. (1989). Interaction of a putative repressor protein with an extended control region of the Bacillus subtilis pur operon. J. Biol. Chem. 264, 3553-3561.
Exterkate, F. A. (1985). A dual directed control of cell wall proteinase production in S. cremoris AM1: a possible mechanism of regulation during growth in milk. Journal of Dairy Science 68, 562-571.
Fisher, S. H., Rohrer, K., and Ferson, A. E. (1996) Role of CodY in regulation of the Bacillus subtilis hut operon J. Bacteriol. 178: 3779-3784.
Foucaud, C., Kunji, E. R., Hagting, A., Richard, J., Konings, W. N., Desmazeaud, M. and Poolman, B. (1995). Specificity of peptide transport systems in Lactococcus lactis: evidence for a third system which transports hydrophobic di- and tripeptides. J. Bacteriol. 177, 4652-4657.
Gajic, O., Kojic, M., Banina, A. and Topisirovic, L. (1999). Characterization of natural isolate Lactococcus lactis subsp. lactis BGMN1-5, a strain producing two bacteriocins, cell wall-associated proteinase and showing clumping phenotype. Arch. Biol. Sci. Belgrade 51, 69-78.
Gasson, M. J. (1983). Plasmid complements of Streptococcus lactis NCDO 712 and other lactic streptococci after protoplast-induced curing. J. Bacteriol. 154, 1-9.
Guedon, E., Renault, P., Ehrlich, S. D., and Delorme, C. (2001a) Transcriptional pattern of genes coding for the proteolytic system of Lactococcus lactis and evidence for coordinated regulation of key enzymes by peptide supply J. Bacteriol. 183: 3614-3622.
Guedon, E., Serror, P., Ehrlich, S. D., Renault, P., and Delorme, C. (2001b) Pleiotropic transcriptional repressor CodY senses the intracellular pool of branched-chain amino acids in Lactococcus lactis Mol. Microbiol. 40: 1227-1239.
Haandrikman, A. J. (1990). Maturation of the cell envelope-associated proteinase of Lactococcus lactis. 64-71.
Hamoen, L. W., A. F. Van Werkhoven, J. J. Bijlsma, D. Dubnau, and G. Venema. 1998. The competence transcription factor of Bacillus subtilis recognizes short A/T-rich sequences arranged in a unique, flexible pattern along the DNA helix. Genes Dev. 12:1539-1550.

Israelsen, H., S. M. Madsen, A. Vrang, E. B. Hansen, and E. Johansen. 1995. Cloning and partial characterization of regulated promoters from Lactococcus lactis Tn917-lacZ integrants with the new promoter probe vector, pAK80. Appl. Environ. Microbiol. 61:2540- 2547.
Kiwaki, M., Ikemura, H., Shimizu-Kadota, M. and Hirashima, A. (1989b). Molecular characterization of a cell wall-associated proteinase gene from Streptococcus lactis NCDO763. Mol. Microbiol. 3, 359-369.
Kok, J., van Dijl, J. M., van der Vossen, J. M. and Venema, G. (1985b). Cloning and expression of a Streptococcus cremoris proteinase in Bacillus subtilis and Streptococcus lactis. Appl. Environ. Microbiol. 50, 94-101.
Kok, J., Leenhouts, K. J., Haandrikman, A. J., Ledeboer, A. M. and Venema, G. (1988a). Nucleotide sequence of the cell wall proteinase gene of Streptococcus cremoris Wg2. Appl. Environ. Microbiol. 54, 231-238.
Kok, J. (1990). Genetics of the proteolytic system of lactic acid bacteria. FEMS Microbiol. Rev. 7, 15-42.
Kok, J. and de Vos, W. M. (1993) The proteolytic system of lactic acid bacteria. London: Blackie and professional, pp. 169-210.
Kuipers, O. P., Beerthuyzen, M. M., Siezen, R. J. and de Vos, W. M. (1993b). Characterization of the nisin gene cluster nisABTCIPR of Lactococcus lactis. Requirement of expression of the nisA and nisI genes for development of immunity. Eur. J. Biochem. 216, 281-291.
Kuipers, O. P., de Ruyter, P. G., Kleerebezem, M., and Vos, W. M. (1998) Qurum sensing controlled gene expression in lactic acid bacteria Biotechnol 64: 15-21.
Kunji, E. R., Mierau, I., Hagting, A., Poolman, B. and Konings, W. N. (1996). The proteolytic systems of lactic acid bacteria. Antonie Van Leeuwenhoek 70, 187-221.
Laan, H., Bolhuis, H., Poolman, B., Abee, T. and Konings, W. N. (1993). Regulation of proteinase synthesis in Lactococcus lactis. Acta Biotechnology 13, 95-101.
Laemmi, U. K. 1970. Cleavage of structural proteins during the assembly of the head of bacteriophage T4. Nature 227:680-685.
Leenhouts, K (1995) Integration strategies and vectors. Dev. Biol. Stand. 85: 523-530.
Leenhouts, K., Buist, G., Bolhuis, A., ten Berge, A., Kiel, J., Mierau, I., Dabrowska, M., Venema, G., and Kok, J. (1996) A general system for generating unlabelled gene replacements in bacterial chromosomes Mol. Gen. Genet. 253: 217-224.
Leenhouts, K., Bolhuis, A., Venema, G. and Kok, J. (1998b). Construction of a food-grade multiple-copy integration system for Lactococcus lactis. Appl. Microbiol. Biotechnol. 49, 417-423.
Marugg, J. D., Meijer, W., van Kranenburg, R., Laverman, P., Bruinenberg, P. G. and de Vos, W. M. (1995). Medium-dependent regulation of proteinase gene expression in Lactococcus lactis: control of transcription initiation by specific dipeptides. J. Bacteriol. 177, 2982-2989.
Marugg, J. D., van Kranenburg, R., Laverman, P., Rutten, G. A., and de Vos, W. M. (1996) Identical transcriptional control of the divergently transcribed prtP and prtM genes that are required for proteinase production in lactococcus lactis SK11 J. Bacteriol. 178: 1525-1531.
Meijer, W. C., Marrug, J. D. and Hugenholtz, J. (1996). Regulation of proteolytic enzyme activity in Lactococcus lactis. Appl. Environ. Microbiol. 62, 156-161.
Mierau, I., Haandrikman, A. J., Velterop, O., Tan, P. S., Leenhouts, K. L., Konings, W. N., Venema, G. and Kok, J. (1994). Tripeptidase gene (pepT) of Lactococcus lactis: molecular cloning and nucleotide sequencing of pepT and construction of a chromosomal deletion mutant. J. Bacteriol. 176, 2854-2861.
Miladinov, N., Kuipers, O. P., and Topisirovic, L. (2001) Casitone-mediated expression of the prtP and prtM genes in Lactococcus lactis subsp. lactis BGIS29 Arch. Microbiol. 177: 54-61.
Miller, J. H. 1972. Experiments in Molecular Genetics. Cold Spring Harbor Laboratory Press, Cold Spring Harbor, N.Y.
Otto, R., de Vos, W. M. and Gavrieli, J. (1982). Plasmid DNA in Streptococcus cremoris Wg2: influence of pH on selection in chemostats of a varient lacking a protease plasmid. Appl. Envion. Microbiol. 43, 1272-1277.
Poolman, B., Konings, W. N. (1988) Relation of growth of Streptococcus lactis and Streptococcus cremoris to amino acid transport J. Bacteriol. 170: 700-707.
Ratnayake-Lecamwasam, M., Serror, P., Wong, K. W., and Sonenshein, A. L. (2001) Bacillus subtilis CodY represses early-stationary-phase genes by sensing GTP levels Genes Dev. 15: 1093-1103.
de Ruyter, P. G., Kuipers, O. P., and de Vos, W. M. (1996) Controlled gene expression systems for Lactococcus lactis with the food-grade inducer nisin Appl. Environ. Microbiol. 62: 3662-3667.
Sambrook, J., Fritsch, E. F., and Maniatis, T. (1989) Molecular cloning: a laboratory manual Cold Spring Harbor, N.Y.: Cold Spring Harbor laboratory Press.
Sanders, J. W., Venema, G., Kok, J., and Leenhouts, K. (1998) Identification of a sodium chloride-regulated promoter in Lactococcus lactis by single-copy chromosomal fusion with a reporter gene Mol. Gen. Genet. 257: 681-685.
Sanz, Y., Lanfermeijer, F. C., Renault, P., Bolotin, A., Konings, W. N., and Poolman, B. (2001) Genetic and functional characterization of dpp genes encoding a dipeptide transport system in Lactococcus lactis Arch. Microbiol. 175: 334-343.
Serror, P., Sonenshein, A. L. (1996a) CodY is required for nutritional repression of Bacillus subtilis genetic competence J. Bacteriol. 178: 5910-5915.
Serror, P., Sonenshein, A. L. (1996b) Interaction of CodY, a novel Bacillus subtilis DNA-binding protein, with the dpp promoter region Mol. Microbiol. 20: 843-852.
Spee, J. H., de Vos, W. M., and Kuipers, O. P. (1993) Efficient random mutagenesis method with adjustable mutation frequency by use of PCR and dITP Nucleic Acids Res. 21: 777-778.
Tynkkynen, S., Buist, G., Kunji, E., Kok, J., Poolman, B., Venema, G., and Haandrikman, A. (1993) Genetic and biochemical characterization of the oligopeptide transport system of Lactococcus lactis J. Bacteriol. 175: 7523-7532.
van Asseldonk, M., A. Simons, H. Visser, W. M. de Vos, and G. Simons. 1993. Cloning, nucleotide sequence, and regulatory analysis of the Lactococcus lactis dnaJ gene. J. Bacteriol. 175:1637-1644.
Visser, S., Exterkate, F. A., Slangen, K. J. and De Veer, G. J. C. M. (1986). Comparative study of the action of cell wall proteinases from various strains of Streptococcus cremoris on bovine aₛ₁- b- and k-casein. Appl. Environ. Microbiol. 52, 1162-1166.
de Vos, W. M., Vos, P., de Haard, H. and Boerrigter, I. (1989). Cloning and expression of the Lactococcus lactis subsp. cremoris SK11 gene encoding an extracellular serine proteinase. Gene 85, 169-176.

1. A method for regulating the expression of a gene of interest in a host cell that comprises a CodY-like protein comprising providing said cell with a gene of interest in operable linkage with a promoter and at least one CodY target sequence.

2. A method according to claim 1, wherein said promoter and/or said CodY target sequence is heterologous with regard to said gene of interest.

3. The method according to claim 1, wherein said CodY target sequence is heterologous with regard to said promoter.

4. The method according to claim 1, wherein said gene of interest is a gene from a gram-positive bacterium.

5. The method according to claim 1, wherein said gene of interest encodes a protease or a peptidase or an anti-microbial peptide or a vitamin.

6. The method according to claim 1, wherein said CodY target sequence comprises a sequence as depicted in FIG. 6A or a functional equivalent and/or a functional fragment thereof.

7. The method according to claim 1, wherein said CodY target sequence comprises a sequence as depicted in FIG. 6B or a functional equivalent and/or a functional fragment thereof.

8. The method according to claim 1, wherein said CodY target sequence comprises a sequence as depicted in Table 4, Table 4A, Table 5, Table 6, Table 7and/or Table 8, or a functional equivalent and/or a functional fragment thereof.

9. The method according to claim 1, wherein said CodY target sequence comprises an ATGTTCA sequence and an inversely repeated ATGTTCA sequence.

10. The method according to claim 9, wherein said nucleic acid sequence comprises a spacing of about 9 base pairs between said ATGTTCA sequence and said inversely repeated ATGTTCA sequence.

11. The method according to claim 9, wherein said nucleic acid sequence comprises the sequence ATGTTCAGAAAATTCATGAACAT.

12. The method according to claim 1, further comprising influencing the binding between said CodY-like protein and said at least one CodY target sequence.

13. A The method according to claim 12, wherein said binding is regulated by subjecting said cell to a change in a growth condition.

14. The method according to claim 12, wherein said binding is regulated by subjecting said cell to a growth limiting condition.

15. The method according to claim 14, wherein said growth limiting condition is a limited availability of a nitrogen source.

16. The method according to claim 1, wherein said host cell is a cell from a (dairy) food production species.

17. The method according to claim 16, wherein said species is selected from a Lactococcus or Lactobacillus or Streptococcus or Leuconostocor Pediococcus or Bifidobacterium or Carnobacterium or Propionibacterium.

18. The method according to claim 1, wherein said host cell is provided with a nucleic acid encoding a CodY-like protein.

19. An isolated or recombinant nucleic acid that comprises at least one CodY target sequence or a functional fragment and/or a functional equivalent thereof.

20. The nucleic acid according to claim 19, further comprising a promoter in operable linkage with a gene of interest.

21. The nucleic acid according to claim 19 further comprising a gene encoding a CodY-like protein.

22. The nucleic acid according to claim 20, wherein said promoter and/or said at least one CodY target sequence is heterologous with regard to said gene of interest.

23. The nucleic acid according to any one of claims 20, wherein said CodY target sequence is heterologous with regard to said promoter.

24. The nucleic acid according to claim 20, wherein said gene of interest is a gene from a gram-positive bacterium.

25. The nucleic acid according to claim 20, wherein said gene of interest encodes a protease or a peptidase or an anti-microbial peptide or a vitamin.

26. The nucleic acid according to claim 19, wherein said CodY target sequence comprises a sequence as depicted in FIG. 6A or a functional equivalent and/or a functional fragment thereof.

27. The nucleic acid according to claim 19, wherein said CodY target sequence comprises a sequence as depicted in FIG. 6B or a functional equivalent and/or a functional fragment thereof.

28. The nucleic acid according to claim 19, wherein said CodY target sequence comprises a sequence as depicted in Table 4, Table 4A, Table 5, Table 6, Table 7 and/or Table 8, or a functional equivalent and/or a functional fragment thereof.

29. The nucleic acid according to claim 19, wherein said CodY target sequence comprises an ATGTTCA sequence and an inversely repeated ATGTTCA sequence.

30. The nucleic acid according to claim 29, wherein said nucleic acid sequence comprises a spacing of about 9 base pairs between said ATGTTCA sequence and said inversely repeated ATGTTCA sequence.

31. The nucleic acid according to claim 29, wherein said nucleic acid sequence comprises the sequence ATGTTCAGAAAATTCATGAACAT.

32. A vector comprising a nucleic acid according to claim 19.

33. A gene delivery vehicle comprising a nucleic acid according to claim 19 or a vector according to claim 32.

34. A host cell comprising a nucleic acid according to claim 19, a vector according to claim 32 or a gene delivery vehicle according to claim 33.

35. A host cell according to 34 which is a cell from a (dairy) food production species.

36. The host cell according to claim 34, wherein said species is selected from a Lactococcus or Lactobacillus or Streptococcus or Leuconostoc or Pediococcus or Bifidobacterium or Carnobacterium or Propionibacterium.

37. A method for using at least one CodY target sequence for regulating the expression of a gene of interest.

38. The method according to claim 37, wherein said target sequence comprises a sequence as depicted in FIG. 6A, FIG. 6B, Table 4, Table 4A, Table 5, Table 6, Table 7 and/or Table 8, or a functional equivalent and/or a functional fragment thereof.

39. The method according to claim 37, wherein said CodY target sequence comprises an ATGTTCA sequence and an inversely repeated ATGTTCA sequence.

40. The method according to claim 37, wherein said nucleic acid sequence comprises a spacing of about 9 base pairs between said ATGTTCA sequence and said inversely repeated ATGTTCA sequence.

41. The method according to claim 37, wherein said nucleic acid sequence comprises the sequence ATGTTCAGAAAATTCATGAACAT.

42. A method for producing a (dairy) food product comprising a step wherein a nucleic acid according to claim 19, a vector according to claim 32, a gene delivery vehicle according to claim 33 or a host cell according to claim 34 is used.

43. The method according to claim 42, wherein said dairy product is a cheese or a fermented milk product.

44. A cheese or a fermented milk product obtainable by a method according to claim 42.

45. A method for at least in part preventing the formation of off-flavours during a process for the production of a (dairy) food product, comprising providing at least one CodY target sequence upstream of a gene which product is, directly or indirectly, involved in the formation of off-flavours.

46. A method for using a nucleic acid according to claim 19, or a vector according to claim 32 or a gene delivery vehicle according to claim 33 or a host cell according to claim 34 for increasing the expression of a gene of interest in a stationary phase culture or equivalents of said culture.

47. The method according to claim 46, wherein said gene of interest comprises a gene encoding an antimicrobial substance, such as a bacteriocin.

48. The method according to claim 46, wherein said gene of interest comprises a gene encoding a flavour compound, vitamin, or a proteinaceous molecule involved in cell lysis.

49. A method for using a nucleic acid according to claim 19 or a vector according to claim 32 or a gene delivery vehicle according to claim 33 or a host cell according to claim 34 for decreasing the expression of a gene in a stationary phase culture or equivalents of said culture.

50. The method according to claim 49, wherein an antisense nucleic acid sequence of an undesired gene is provided in operable linkage with a promoter and at least one CodY target sequence.

51. The method according to claim 49, wherein said gene is involved with acidification.
